(19) United States
(12) Patent Application Publication
(10) Pub. No.: US 2022/0233641 A1
(43) Pub. Date: Jul. 28, 2022

(54) Methods and Compositions for Treating Obesity and/or Skin Disorders

(71) Applicant: THE TRUSTEES OF THE UNIVERSITY OF PENNSYLVANIA, Philadelphia, PA (US)

(72) Inventors: Taku KAMBAYASHI, Malvern, PA (US); Ruth CHOA, Ellicott City, MD (US)

(21) Appl. No.: 17/611,309

(22) PCT Filed: May 18, 2020
(86) PCT No.: PCT/US20/33415

§ 371 Date: Nov. 15, 2021

Related U.S. Application Data
(60) Provisional application No. 62/849,656, filed on May 17, 2019.
(60) Provisional application No. 62/972,462, filed on Feb. 10, 2020.

(51) Int. Cl. A61K 38/19; A61P 3/04; A61P 1/16; A61K 31/593; C12N 15/86

(52) U.S. Cl. CPC A61K 38/19; A61P 3/04; A61P 1/16; C12N 2750/14171; C12N 15/86; C12N 2750/14143; A61K 31/593

(57) ABSTRACT

Compositions and methods for treating and/or ameliorating obesity are provided. In certain embodiments, the compositions comprise the topical vitamin D3 derivative MC903. In other embodiments, the compositions systemically increase TSLP levels in a subject. In yet other embodiments, the compositions include TSLP peptide isoforms and/or adeno-associated viral vectors containing TSLP-expressing sequences. Methods using these compositions increase TSLP levels in the subject and cause selective loss of white adipose tissue without loss in muscle mass.

### CROSS-REFERENCE TO RELATED APPLICATIONS

The present application claims priority under 35 U.S.C. § 119(e) to U.S. Provisional Applications No. 62/849,656 filed May 17, 2019 and No. 62/972,462 filed Feb. 10, 2020, each of which application is hereby incorporated by reference in its entirety herein.

### STATEMENT REGARDING FEDERALLY SPONSORED RESEARCH

This invention was made with government support under HL111501 awarded by the National Institutes of Health. The government has certain rights in the invention.

### BACKGROUND

Obesity and obesity-related disorders such as type 2 diabetes (T2D) and non-alcoholic steatohepatitis (NASH) are major health threats around the world, especially in developed countries. Other than lifestyle modifications and major surgical procedures, there are not many treatment options available for these conditions.

There is thus a need in the art for novel compositions and/or methods that allow for controlled weight loss. The present invention addresses this need.

### BRIEF SUMMARY OF THE INVENTION

The present invention provides methods of treating obesity and/or an obesity-related disorder. The present invention further provides a method of treating a skin disorder and/or improving scalp health. The present invention further provides a method of treating an eye disorder. The present invention further provides a method of treating, ameliorating, and/or preventing a skin disorder. The present invention further provides a method of treating, ameliorating, and/or preventing alopecia in a subject.

In certain embodiments, the method comprises topically administering to a subject in need thereof a pharmaceutically effective amount of a vitamin D₃ analog. In certain embodiments, the method comprises administering to a subject in need thereof a pharmaceutically effective amount of a TSLP inhibiting agent. Further non-limiting embodiments defined the present invention are recited elsewhere herein.

### BRIEF DESCRIPTION OF THE FIGURES

The drawings illustrate generally, by way of example, but not by way of limitation, various embodiments of the present application.

FIGS. 1A-1D illustrate prevention weight gain and improvement in metabolic parameters of HFD-fed mice as a result of administering MC903. WT C57BL/6 mice were fed a normal chow (NC) or 40% high fat diet (HFD) for 12 weeks. The mice were topically treated with either vehicle (EtOH) or MC903 (2 nmol/ear) on both ears Monday, Wednesday, and Friday on odd weeks. (FIG. 1A) Mice were weighed weekly and % change from baseline is plotted as mean±SEM. (FIG. 1B) The mice were euthanized on Week 12 and the epididymal fat pads were weighed. (FIG. 1C, FIG. 1D) Glucose tolerance test was performed on Week 12 by injection of glucose and subsequent measurements of blood glucose levels. HOMA-IR analysis was performed after an overnight fast at Week 12. *, **, and *** indicate statistical significance of p<0.05, p<0.01, and p<0.001, respectively by Student t-test or ANOVA.

FIG. 2 illustrates prevention of HFD-induced hepatosteatosis by MC903. WT C57BL/6 mice were fed a normal chow (NC) or 40% high fat diet (HFD) for 12 weeks. The mice were topically treated with either vehicle (EtOH) or MC903 (2 nmol/ear) on both ears Monday, Wednesday, and Friday on odd weeks. Shown are representative histological images of the liver on Week 12.

FIG. 3 shows that MC903 does not prevent weight gain or improve metabolic parameters in HFD-fed TSLP-R KO mice. TSLP-R KO mice were fed a normal chow (NC) or 40% high fat diet (HFD) for 12 weeks. The mice were topically treated with either vehicle (EtOH) or MC903 (2 nmol/ear) on both ears Monday, Wednesday, and Friday on odd weeks. Mice were weighed on the indicated time points and plotted as mean±SEM. Glucose tolerance test was performed on Week 12 by injection of glucose and subsequent measurements of blood glucose levels. AUC analysis is performed on the GTT curves. * indicates statistical significance of p<0.05 by Student t-test.

FIG. 4 illustrates AAV8-TSLP induces weight loss in HFD-fed mice. WT C57BL/6 mice were fed a 40% high fat diet (HFD) for 4 weeks. The mice were injected with either AAV8-control or AAV8-TSLP on Day 0. The mice were weighed weekly and % change from baseline is plotted as mean±SEM. ** and *** indicate statistical significance of p<0.01 and p<0.001, respectively.

FIG. 5 illustrates AAV8-TSLP induces weight loss in previously obese HFD-fed mice. WT C57BL/6 mice were fed a 40% high fat diet (HFD) for 10 weeks. The mice were then injected with either AAV8-control or AAV8-TSLP at Week 10. The mice were kept on a HFD and weighed weekly and % change from baseline is plotted as mean±SEM. *, **, and *** indicate statistical significance of p<0.05, p<0.01, and p<0.001, respectively by Student t-test or ANOVA.

FIG. 6 illustrates AAV8-TSLP induces loss of subcutaneous fat. WT C57BL/6 mice were fed a 40% high fat diet (HFD) for 10 weeks. The mice were then injected with either AAV8-control or AAV8-TSLP at Week 10. The mice were kept on a HFD for 6 weeks and euthanized for histological analysis. Shown are representative histological images of the skin of mice.

FIG. 7 illustrates AAV8-TSLP induces weight loss in Ob/Ob mice. Ob/Ob mice were injected with either AAV8-control or AAV8-TSLP and fed a normal chow for 6 weeks. The mice weighed weekly and % change from baseline is plotted as mean±SEM. * and *** indicate statistical significance of p<0.05 and p<0.001, respectively by Student t-test.

FIGS. 8A-8E illustrate AAV8-TSLP induces selective adipose tissue loss in normal chow-fed mice. WT C57BL/6 mice were injected with either AAV8-control or AAV8-TSLP and fed a normal chow for 14 days. (FIG. 8A) Mice were weighed on the indicated days and % change from baseline is plotted as mean±SEM. The mice were euthanized on the indicated days and the (FIG. 8B) eWAT, (FIG. 8C) iWAT, (FIG. 8D) BAT, and (FIG. 8E) quadriceps weights were measured. ** and *** indicate statistical significance of p<0.01 and p<0.001, respectively by Student t-test.

FIGS. 9A-9C illustrate AAV8-TSLP does not alter food consumption of gut absorption. WT C57BL/6 mice were injected with either AAV8-control or AAV8-TSLP and fed a normal chow or HFD for 12 weeks. (FIG. 9A) Average weekly food consumption is plotted as mean±SEM. On day 10 post AAV8 injection, mice were oral gavaged with a fixed amount of (FIG. 9B) glucose or (FIG. 9C) olive oil. Subsequent glucose levels and triglyceride levels were measured and plotted over time.

FIGS. 10A-10B illustrate AAV8-TSLP-treated mice excrete less fecal energy compared to AAV8-control-treated mice. WT C57BL/6 mice were injected with either AAV8-control or AAV8-TSLP and fed a normal chow for 9 days. Stool was collected and food consumption was measured individually caged mice between days 9 and 11. The collected stool was subjected to bomb calorimetry for measurement of fecal calories. (FIG. 10A) The fecal energy content per day of AAV8-control or AAV8-TSLP treated mice is shown. (FIG. 10B) The net energy intake per day, as calculated by the food caloric intake subtracted by the fecal calories of AAV8-control or AAV8-TSLP treated mice is shown. Statistical analysis performed by Student t-test.

FIG. 11 illustrates that AAV8-TSLP increases food consumption between Days 7-10 post injection. WT C57BL/6 mice were injected with either AAV8-control or AAV8-TSLP and placed in a metabolic chamber on Days 7-10. Hourly and cumulative food consumption is plotted over the last 48 hours.

FIG. 12 illustrates that AAV8-TSLP does not increase oxygen consumption between Days 7-10 post injection. WT C57BL/6 mice were injected with either AAV8-control or AAV8-TSLP and placed in a metabolic chamber on Days 7-10. Hourly (left plot) and daily/half daily (right plot) oxygen consumption is plotted over the last 48 hours and 72 hours, respectively.

FIG. 13 illustrates that AAV8-TSLP does not increase carbon dioxide output between Days 7-10 post injection. WT C57BL/6 mice were injected with either AAV8-control or AAV8-TSLP and placed in a metabolic chamber on Days 7-10. Hourly (left plot) and daily/half daily (right plot) carbon dioxide output is plotted over the last 48 hours and 72 hours, respectively.

FIG. 14 illustrates that AAV8-TSLP does not increase locomotor activity between Days 7-10 post injection. WT C57BL/6 mice were injected with either AAV8-control or AAV8-TSLP and placed in a metabolic chamber on Days 7-10. Hourly (left plot) and daily/half daily (right plot) locomotor activity is plotted over the last 48 hours and 72 hours, respectively.

FIG. 15 illustrates that AAV8-TSLP causes oily fur in previously obese HFD-fed mice. WT C57BL/6 mice were fed a 40% high fat diet (HFD) for 10 weeks. The mice were then injected with AAV8-TSLP at Week 10. The mice were kept on a HFD for 6 weeks. A representative photograph of the mice on Week 6 is shown.

FIG. 16 illustrates that AAV8-TSLP does not induce adipose tissue loss in normal chow-fed SCD1 KO mice. WT C57BL/6 or SCD1 KO mice were injected with either AAV8-control or AAV8-TSLP and fed a normal chow for 14 days. The mice were euthanized and the eWAT, weight was measured. Statistical analysis performed by Student t-test.

FIGS. 17A-17B illustrate AAV8-TSLP induces weight loss in a T cell-dependent manner. (FIG. 17A) RAG KO mice and (FIG. 17B) TCR-beta KO mice were injected with either AAV8-control or AAV8-TSLP. The epididymal white adipose tissue (eWAT) was weighed on Day 14 post injection. Data are plotted as mean±SEM.

FIG. 18 illustrates, without being bound by theory, a non-limiting proposed model of TSLP-induced adipose tissue loss. The elevation of systemic TSLP can be accomplished by topical treatment with MC903, injection of recombinant TSLP, or gene therapy using a viral vector. Increased systemic TSLP levels either directly or indirectly leads to skin lipid secretion, which increases the fat energy demands of the organism. This causes liberation of fat stores (lipolysis) in adipose and other fat-harboring tissues. This eventually leads to weight loss and reversal of obesity.

FIG. 19 illustrates the finding that subcutaneous fat is markedly reduced in mice expressing high levels of TSLP.

FIG. 20 illustrates the finding that visceral fat is markedly reduced in mice expressing high levels of TSLP in a dose-dependent manner.

FIG. 21 shows that mice injected with AAV8-TSLP display increased sebum components on their fur. WT C57BL/6 mice were injected with either AAV8-control or AAV8-TSLP and fed a normal chow for 10 days. A fixed area of back fur was shaved from the mice and lipids were extracted. The extracted lipids were subjected to thin layer chromatography and the lipid components were separated into wax monoesters (WME), wax diesters (WDE), free fatty acids (FFA), and free cholesterol (FC). The amount was quantified by Image J software and plotted. ** indicates statistical significance of p<0.01 by Student t-test.

FIG. 22 shows that mice injected with AAV8-TSLP display increased Ki67+ basal layer sebocytes. WT C57BL/6 mice were injected with either AAV8-control or AAV8-TSLP and fed a normal chow for 10 days. The skin was removed and fixed. Sections from paraffin-embedded skin tissue were stained with anti-Ki67 antibodies. The number of Ki67+ basal layer sebocytes were counted and the fraction of Ki67+/total sebocytes was calculated. ** indicates statistical significance of p<0.01 by Student t-test.

FIG. 23 shows that TSLP-R KO mice display decreased sebum lipid components on their fur compared to WT mice. A fixed area of back fur was shaved from WT C57BL/6 mice or TSLP-R KO mice and lipids were extracted. The extracted lipids were subjected to thin layer chromatography and the lipid components were separated into wax monoesters (WME), wax diesters (WDE), free fatty acids (FFA), and free cholesterol (FC). The amount was quantified by Image J software and plotted. ** indicates statistical significance of p<0.01 by Student t-test.

FIG. 24 shows that mice injected with AAV8-TSLP have increased expression of anti-microbial peptides in their skin. WT C57BL/6 mice were injected with either AAV8-control or AAV8-TSLP and fed a normal chow for 10 days. The skin was removed and RNA was extracted. Transcript levels of cathelicidin antimicrobial peptide (CAMP) and defensin B4 (DEF4B), two antimicrobial peptides associated with sebum, were measured by qPCR. * indicates statistical significance of p<0.05 by Student t-test.

FIG. 25 illustrates that topical application of MC903 remotely increases sebum secretion in a human individual. MC903 was applied topically to the right arm of an individual daily. Sebum measurements were obtained by cleaning the forehead with ethanol wipe, resting for 15 minutes, and placing sebutape on the forehead for 2 minutes. Black dots on the blue and white background of the tape indicates the presence of sebum at each day post treatment.

FIG. 26 illustrates that the topical application of MC903 remotely cures dry skin of the hand in a human individual. MC903 was applied topically to the right arm of an individual daily. Photographs of the right hand before and after 3 or 5 days post treatment is shown. The individual was treated for 14 days total with no need of hand cream at least 9 weeks even after treatment was discontinued. In certain embodiments, secretion of sebum lasts after cessation of treatment.

FIG. 27 illustrates that the topical application of MC903 remotely promotes scalp hair growth in a human individual. MC903 was applied topically to the left arm daily of an individual for 14 days (1^{st} treatment). Treatment was stopped for 4 weeks and restarted for an additional 2 weeks (2^{nd} treatment) on the right arm daily. Photographs of the scalp before, 2 weeks after 1^{st} treatment, 11 days after 2^{nd} treatment, and 8 weeks after 2^{nd} treatment are shown.and after 3 or 5 days post treatment is shown.

FIG. 28 shows that the topical application of MC903 remotely improves dry eye symptoms in two human individuals. MC903 was applied topically to the left arm daily of an individual and the right leg another individual for 14 days (1^{st} treatment). The presence of dry eye symptoms was subjectively evaluated in the two individuals before and after treatment. The response is plotted in a bar graph.

FIGS. 29A-29F illustrate that 1 TSLP induces selective white adipose loss and reverses obesity and associated metabolic complications. FIG. 29A: Weights of HFD-fed mice post AAV (Control-AAV or TSLP-AAV). (n=5 mice/group). (FIG. 29B) Weights, (FIG. 29C) glucose tolerance test (GTT), and (FIG. 29D) liver TGs of obese mice (10w on HFD before AAV, n=5 NC, 10 HFD-Ctrl, and 8 HFD-TSLP mice). FIG. 29E: Serum ALT of MCDD-fed mice 325 injected with AAV (n=7 mice/group). FIG. 29F: Tissue masses of NC-fed mice 2w post AAV (n=6 mice/group). Data are mean±s.e.m. from 2 independent experiments. ANOVA (FIGS. 29A-29C) or Student's t-test (FIGS. 29D-29F). Statistics: (FIG. 29A) F=18.96, dfₙ=1, df_{d}=8. (FIG. 29B) F=32.29, dfₙ=1, df_{d}=16. (FIG. 29C) NC vs. HFD-Ctrl: F=14.33, dfₙ=1, df_{d}=13, NC vs. HFD-TSLP: F=1.753, dfₙ=1, df_{d}=11, HFD-Ctrl vs. HFD-TSLP: F=27.71, dfₙ=1, df_{d}=16. (FIG. 29D) t=9.835, df=23. (FIG. 29E) t=2.560, df=12. (FIG. 29F) eWAT: t=8.128, iWAT: t=7.226, BAT: t=1.150, and Muscle: t=0.2566, with df=10 for all tissues.

FIGS. 30A-30F illustrate TSLPR signaling in T cells is required for TSLP-driven adipose loss. eWAT masses of (FIG. 30A) E-Beta KO mice (n=5 mice/group), (FIG. 30B) aCD4±aCD8-injected WT mice (n=4 Ctrl and 6 TSLP mice for all groups), (FIGS. 30C-30D) RAG KO mice transferred with CD4+ or CD8+ T cells (n 5 mice/group for all groups except CD8+ T transferred-TSLP mice, where n=6 mice) and WT or TSLPR KO T cells (n 8 WT T and 10 TSLPR KO T transferred mice), and (FIG. 30E) TSLPR. KO mice transferred with WT or TSLPR KO T cells (n=8 mice/group) 2w post AAV. (FIG. 30F) eWAT mass of AAV-injected WT mice before antibody injection (2w post AAV, n=4 mice/group), 2w after isotype (4w post AAV, n=5 mice/group) or aCD4+aCD8 antibody injection (4w post AAV, n=8 Ctrl and 7 TSLP mice). Data are mean±s.e.m. from 2 (FIGS. 30A-C, 30E-30F) or 3 (FIG. 30D) independent experiments. Student's t-test. Statistics: (FIG. 30A) t=0.0466, df=8. (FIG. 30B) aCD4: t=6.231, df=8, aCD8: t=5.032, df=8, and aCD4±aCD8: t=0.1433, df=8. (FIG. 30C) CD4+: t=8.787, df=8, CD8+: t=3,598, df=9. (FIG. 30D) WT: t=7.103, df=11.2, TSLPR KO: t=0.2950, df=18. (FIG. 30E) WT: t=6.255, df=14, TSLPR KO: 0.8481, df=14. (FIG. 30F) Before Ab: t=3,994, df=6, Isotype Ab: t=34.95, df=8, aCD4+aCD8: t=4.641, df=13, Before Ab TSLP vs. aCD4+aCD8 TSLP: t=3.151=9, Isotype Ab TSLP vs. aCD4+aCD8 TSLP: t=11.17, df=10.

FIGS. 31A-31J illustrate that TSLP induces adipose loss by promoting T cell-mediated sebum secretion. (FIG. 31A) Food consumption over 3 days (n=7 Ctrl and 8 TSLP mice), (FIG. 31B) fecal calories (n=5 mice/group), and (FIG. 31C) energy expenditure (n=7 Ctrl and 8 TSLP mice) of NC-fed mice 9-11d 3 post AAV. (FIG. 31D) Gross appearance and (FIG. 31E) TLC quantification of HFD-fed mice at 4w post AAV (CE=cholesterol esters, WE=wax esters, TG=triglycerides, FFA=free fatty acids, FC=free cholesterol, n=9 mice/group). (FIGS. 31F-31G) Sebaceous gland Ki67 staining and quantification of NC-fed mice 10d post AAV. (n=78 Ctrl and 50 TSLP sebaceous glands from 3 mice/group). (FIG. 31H) TLC quantification of fur lipids from WT and RAG KO mice 2w post AAV (n=5 mice/group). (FIG. 31I) Skin CD3 staining 10d post AAV. (FIG. 31J) eWAT masses of WT vs SCD1 KO mice 2w post AAV (n=5 WT mice/group and 6 SCD1 KO mice/group). Data are mean±s.e.m. from 2 independent experiments. Student's t-test (FIGS. 31A-31B, 31E, 31G-31H, 31J) or ANOVA (FIG. 31C). Statistics. (FIG. 31A)t=2.624, df=13. (FIG. 31B) t=3.046, df=9. (FIG. 31C) F=0.9142, dfₙ=1, df_{d}=13. (FIG. 31E) CE: t=4.079, WE: t=2.931, TG: t=3.721, FFA: t=3.346, FC; t=4.273, df=16 for all comparisons. (FIG. 31G) t=3.763, df=12. (FIG. 31H) WT CE: t=2.561, RAG KO CE: t=0.4456, WT WE: t=4.380, RAG KO WE: t=1.163, WT FFA: t=5.333, RAG KO FFA: t=1.990, WT FC: t=3.388, KO FC: t=1.632, df=8 for all comparisons. (FIG. 31J) For WT—Ctrl vs. TSLP: t=4.858, df=8. For SCD1 KO—Ctrl vs TSLP: t=1.440, df=10. For WT—TSLP vs. SCD1 KO—TSLP: t=4.650, df=9.

FIGS. 32A-32F illustrate that TSLP and T cells regulate sebum secretion at homeostasis. (FIG. 32A) TLC quantification of WT vs. TSLPR KO mice (n=11 mice/group). (FIGS. 32B-32C) Ki67 staining and =quantification of WT vs. TSLPR KO sebaceous glands (n=132 WT and 109 TSLPR KO sebaceous glands from 3 mice/group). (FIG. 32D) TLC quantification of WT mice vs. E-Beta KO mice (n=4 WT and 3 E-Beta KO mice). (FIG. 32E) Linear regression analysis of TSLP expression versus sebaceous gland (SG) gene expression in publicly available human skin microarray data. (n=36 healthy individuals). (FIG. 32F) Illustrative non-limiting model for pharmacologic and homeostatic roles of TSLP-driven sebum secretion. Data are mean s.e.m. from 2 independent experiments (FIGS. 32A-32D). Student's t-test (FIGS. 32A, 32C-32D), or Pearson's r and Linear regression slope test (FIG. 32E). Statistics: (FIG. 32A) CE: t=1.530, WE: t=3.924, FFA: t=1.748, FC: t=1.052, df=20 for all comparisons. (FIG. 32C) t=3.139, df=239. (FIG. 32D) CE: t=4.219, WE: t=4.234, FFA: t=1.555, FC: t=1.636, df=5 for all comparisons. (FIG. 32E) F=9.393, dfₙ=1, df_{d}=34.

FIGS. 33A-33J illustrate that TSLP stimulates weight loss and improves metabolic parameters. (FIG. 33A) % Weight change of HFD-fed mice post AAV (n=5 mice/group). (FIG. 33B) Weight and (FIG. 33C) % weight change of NC-fed ob/ob mice post AAV (n=4 mice/group). (FIG. 33D) % Weight change, (n=9 mice/group), (FIG. 33E) eWAT mass (n=9 Ctrl and 8 TSLP mice), (FIG. 33F) fasting glucose (n=8 mice/group), (FIG. 33G) fasting insulin (n=8 mice/group), (FIG. 33H) HOMA-IR (n=8 mice/group), (FIG. 33I) GTT quantification (n=5 NC, 10 HFD-Ctrl, and 8 HFD-TSLP mice), and (FIG. 33J) liver H&E histology of obese mice (previously fed HFD for 10w) 4w post AAV. Data are shown as mean s.e.m. from 2 independent experiments. ANOVA with Sidak's post hoc test (FIGS. 33A-33D) or Student's t-test (FIGS. 33E-33I). Statistics. (FIG. 33A) F=19.27, dfₙ=1, df_{d}=8. (FIG. 33B) F=1.911, dfₙ=1, df_{d}=6. Week 1: t=0.4474, df=4.910, Week 2: t=0.1274, df=4.365, Week 3: t=1.543, df=3.482, Week 4: t=2.524, df=3.217, Week 5: t=3.282, df=3.365. (FIG. 33C) F=5.139, dfₙ=1, df_{d}=6. Week 1: t=1.857, df=4.992, Week 2: t=0.1761, df=4, Week 3: t=2.445, df=5.674, Week 4: t=2.342, df=5.967, Week 5: t=2.833, df=5.914. (FIG. 33D) F=10.05, dfₙ=1, df_{d}=16. (FIG. 33E) t=10.81, df=15. (FIG. 33F) NC vs. HFD-Ctrl: t=6.692, NC vs. HFD-TSLP: t=0.2312, HFD-Ctrl vs. HFD-TSLP: t=6.129, df=14 for all comparisons. (FIG. 33G) NC vs. HFD-Ctrl: t=2.757, fNC vs. HFD-TSLP: t=0.5797, HFD-Ctrl vs. HFD-TSLP: t=3.144, df=14 for all comparisons. (FIG. 33H) NC vs. HFD-Ctrl: t=4.381, NC vs. HFD-TSLP: t=1.899, HFD-Ctrl vs. HFD-TSLP: t=3.783, df=14 for all comparisons. (FIG. 33I) NC vs. HFD-Ctrl: t=3.688, df=13, NC vs. HFD-TSLP: t=1.296, df=11, HFD-Ctrl vs. HFD647 TSLP: t=5.141, df=16.

FIGS. 34A-34L illustrate that TSLP ameliorates MCDD-driven liver damage and induces selective white adipose loss in NC-fed mice. (FIG. 34A) Liver TGs (n=5 NC, 10 MCDD Ctrl, and 10 MCDD TSLP mice), (FIGS. 34B-34D) PicroSiriusRed liver staining and quantification of MCDD-fed mice post AAV (n=15 mice/group). (FIG. 34E) eWAT, (FIG. 34F) iWAT, (FIG. 34G) BAT, and (FIG. 34H) quadriceps muscle mass (n=6 mice/group for all tissues), (FIG. 34I) NMR analysis Pre (Day 0) and Post (Day 14) AAV (n=5 mice/group), (FIG. 34J) BCS (n=10 mice/group), and (FIG. 34K) % weight change (n=6 mice/group) of NC-fed mice 2w post AAV. (FIG. 34L) eWAT mass of TSLPR KO mice 2w post AAV (n=7 mice/group). Data are shown as mean±s.e.m. from 2 (FIGS. 34A, 34E-34H, 34J-34L) or 3 (FIGS. 34C-34D) independent experiments. Student's t-test (FIGS. 34A, 34C-34I, 34L) or ANOVA with Sidak's post hoc test (FIGS. 34J-34K). Statistics. (FIG. 34A) NC vs. MCDD Ctrl: t=9.703, df=13, NC vs. MCDD TSLP: t=13.17, df=13, MCDD Ctrl vs. MCDD TSLP: t=4.768, df=18. (FIGS. 34B-34D) % Area: t=5.399, df=28, Integrated density: t=5.640, df=28. (FIG. 34E) Day 3: t=0.9539, Day 7: t=0.6498, Day 10: t=2.183, Day 14: t=8.128. (FIG. 34F) Day 3: t=0.3344, Day 7: t=0.6961, Day 10: t=5.972, Day 14: t=7.226. (FIG. 34G) Day 3: t=1.355, Day 7: t=0.1674, Day 10: t=2.829, Day 14: t=1.150. (FIG. 34H) Day 3: t=0.4126, Day 7: t=0.0925, Day 10: t=0.5618, Day 14: t=0.2566, df=10 for all comparisons. (FIG. 34I) Ctrl Pre vs. Post: t=0.3241, TSLP Pre vs. Post: t=3.736, Ctrl Post vs. TSLP Post: t=3.111, df=8 for all comparisons. (FIG. 34J) F=0.000, dfₙ=1, df_{d}=18. (FIG. 34K) F=2.635, dfₙ=1, df_{d}=10. (FIG. 34L) t=0.5779, df=12.

FIGS. 35A-35E illustrate that TSLPR signaling in hematopoietic cells is required for TSLP-driven white adipose loss and weight loss. WT or TSLPR KO mice were irradiated and reconstituted with WT or TSLPR KO bone marrow: (FIG. 35A) Schematic and (FIG. 35B) eWAT mass of NC-fed mice 2w post AAV (n=7 WT a WT Ctrl, 9 WT a WT TSLP, 9 KO a WT Ctrl, 9 KO a WT TSLP, 7 WT a KO Ctrl, and 5 WT a KO TSLP mice). (FIGS. 35C-35E) Weight of HFD-fed chimeras post AAV (n=6 WT a WT Ctrl, 10 WT a WT TSLP, 10 KO \dot{a} WT Ctrl, 11 KO a WT TSLP, 11 WT a KO Ctrl, and 14 WT a KO TSLP mice). Data are mean±s.e.m. from 3 independent experiments. Student's t-test (FIG. 35B) or ANOVA with Sidak's post hoc test (FIGS. 35C-35E). Statistics. (FIG. 35B) WT \dot{a} WT: t=3.959, df=14, KW, WT: t=0.0661, df=16, WT a KO: t=4.807, df=10. (FIG. 35C) F=8.120, dfₙ=1, df_{d}=14. (FIG. 35D) F=2.727, dfₙ=1, df_{d}=19. (FIG. 35E) F=22.97, dfₙ=1, df_{d}=23.

FIGS. 36A-36E illustrate that adaptive immune cells, but not DCs, eosinophils, or Treg cells are required for TSLP-driven adipose loss. eWAT masses of (FIG. 36A) ROSA-Stop-flox-DTA mice (n=5 Ctrl and 4 TSLP mice) and CD11cCre-ROSA-Stop-flox-DTA mice (n=5 Ctrl and 6 TSLP mice), (FIG. 36) ROSA-Stop-flox-DTA mice (n=5 Ctrl and 4 TSLP mice) and EoCre-ROSA-Stop-flox-DTA mice (n=6 Ctrl and 6 TSLP mice), (FIG. 36C) FOXP3-GFP and FOXP3-DTR mice treated with PBS or diphtheria toxin (DT) (n=6 mice/group, except for FOXP3-DTR PBS mice, where n=5 mice), (FIG. 36D) RAG/IL2rg DKO mice injected with PBS or WT T cells (n=6 PBS Ctrl, 8 PBS TSLP, 5 T cell Ctrl, and 5 T cell TSLP mice), and (FIG. 36E) RAG KO mice (n=10 mice/group) 2w post AAV. Data are shown as mean±s.e.m. from 2 (FIGS. 36A-36D) or 3 (FIG. 36E) independent experiments. Student's t-test. Statistics. (FIG. 36A) DTA: t=5.135, df=7, CD11cCre-DTA: t=7.114, df=9. (FIG. 36B) DTA: t=5.135, df=7, EoCre-DTA: t=3.928, df=10. (FIG. 36C) FOXP3-GFP PBS: t=5.375, df=10, FOXP3-GFP DT: t=3.954, df=10, FOXP3-DTR PBS: t=6.100, df=9, FOXP3-DTR DT: t=4.593, df=10. (FIG. 36D) PBS: t=0.6571, df=10, T cells: t=7.710, df=6. (FIG. 36E) t=0.4072, df=18.

FIGS. 37A-37L illustrate that TSLP does not affect energy intake, metabolic rate, locomotor activity, or excretion of urine metabolites. NC-fed mice were placed into individual metabolic chambers between 9-11d post AAV, and monitored for the following: (FIG. 37A) Food consumption (n=7 mice/group), (FIGS. 37D-37E) locomotor activity. (FIG. 37F) O₂ consumption, (FIG. 37G) CO₂ production, and (FIG. 37H) respiratory exchange ratio (n=7 Ctrl and 8 TSLP mice). (FIGS. 37B-37C) Plasma glucose and TG levels following oral GTT and OFTT (n=5 mice/group). (FIG. 37I) eWAT masses of WT and UCP1 KO mice 2w post AAV (n=5 mice/group, except UCP1 KO TSLP mice, where n=7 mice). Urine (FIG. 37J) glucose, (FIG. 37K) ketones, and (FIG. 37L) protein 10 days post AAV (n=10 mice/group). Data are mean±s.e.m. from 2 independent experiments. ANOVA (FIGS. 37A-37H) or Student's t-test (FIG. 37I). Statistics. (FIG. 37A) F=0.8248, dfₙ=1, df_{d}=12. (FIG. 37B) F=0.3628, dfₙ=1, df_{d}=8. (FIG. 37C) F=3.811, dfₙ=1, df_{d}=8. (FIG. 37D) F=0.5290, (FIG. 37E) F=0.0148, (FIG. 37F) F=0.0851, (FIG. 37G) F=0.0374, and (FIG. 37H) F=0.8375, dfₙ=1 and df_{d}=12 for all comparisons FIGS. 37D-37H. (FIG. 37I) WT: t=9.401, df=8, UCP1 KO: t=10.26, df=10.

FIGS. 38A-38K illustrate that TSLP increases sebum secretion. (FIG. 38A) Lipid mass and (FIG. 38B) TLC plot from HFD-fed mice 4w post AAV (n=9 mice/group). (FIG. 38C) Lipid mass and (FIGS. 38D-38E) TLC plot and quantification from NC-fed mice 10d post AAV (n=9 mice/group). (FIG. 38F) Back skin H&E histology, sebaceous gland size (red dashed line) 10d post AAV. (FIG. 38G) Gland size quantification (n=101 Ctrl and 100 TSLP sebaceous glands from 3 mice/group). (FIG. 38H) Oil Red 0 (ORO) staining of back skin 10d post AAV. (FIG. 38I) ORO integrated density (n=95 Ctrl and 131 TSLP sebaceous glands from 3 mice/group). (FIGS. 38J-38K) Numbers of Ki67⁺ and Ki67⁻ basal cells in sebaceous glands 10d post AAV (n=78 Ctrl and 50 TSLP sebaceous glands from 3 mice/group). Data are shown as mean±s.e.m. from 2 independent experiments. Student's t-test. Statistics. (FIG. 38A) t=6.897, df=16. (FIG. 38C) t=2.252, df=16. (FIG. 38E) CE: t=0.6720, WE: t=3.829, FFA: t=2.238, FC: t=1.426, df=18 for all comparisons. (FIG. 38G) t=6.749, df=199. (FIG. 38I) t=0.0985, df=232. (FIG. 38J) t=2.863, df=126. (FIG. 38K) t=0.7165, df=126.

FIGS. 39A-39C illustrate that TSLPR signaling in T cells is required for TLSP-driven sebum secretion. (FIG. 39A) TLC plot from WT vs. RAG KO mice 2w post AAV. (FIGS. 39B-39C) TLC plot and quantification from RAG KO mice adoptively transferred with WT or TSLPR KO T cells 2w post AAV (n=6 WT T transferred Ctrl, 9 WT T transferred TSLP, 9 TSLPR KO T transferred Ctrl, and 11 TSLPR KO T transferred TSLP mice). Data are shown as mean±s.e.m. from 2 independent experiments. Student's t-test. Statistics. (FIG. 39C) WT CE: t=1.196, df=13, TSLPR KO CE: t=0.6370, df=18, WT WE: t=\2.695, df=13, TLSPR KO WE: t=1.122, df=18, WT FFA: t=2.057, df=13, TSLPR KOFFA: t=0.6613, df=18, WT FC: t=3.111, df=13, TSLPR KO FC: t=0.6130, df=18.

FIGS. 40A-40I illustrate that TSLP increases T cells in the skin. (FIG. 40A) Representative cytometric flow plots of CD4⁺ and CD8⁺ T cells in the skin 10d post AAV.

(FIGS. 40B-40C) % and number of skin T cells. (FIGS. 40D-40E) % and number of skin CD4⁺ T cells. (FIGS. 40F-40G) % and number of skin CD8⁺ T cells. (n=11 Ctrl and 9 TSLP mice). (FIGS. 40H-40I) CD4 and CD8 staining in the skin 10d post AAV. Data are shown as mean s.e.m. from 2 experiments. Student's t-test. Statistics. (FIG. 40B) t=3.331, (FIG. 40C) t=3.249, (FIG. 40D) t=4.355, (FIG. 40E) t=3.325, (FIG. 40F) t=2.886, (FIG. 40G) t=3.401, df=18 for all comparisons.

FIGS. 41A-41H illustrate that TSLPR KO, RAG KO, and E-Beta KO mice have decreased sebum secretion. (FIG. 41A) Representative TLC plot of fur lipids from WT vs. TSLPR KO mice. (FIG. 41B) Back skin H&E histology of from WT vs. TSLPR KO mice. (FIG. 41C) Quantification of sebaceous gland size (n=106 WT and 95 TSLPR KO sebaceous glands from 3 mice/group). (FIG. 41D) ORO staining of WT vs. TSLPR KO mice back skin and (FIG. 41E) ORO quantification (n=149 WT and 129 TSLPR KO sebaceous glands from 3 mice/group). (FIGS. 41F-41G) Representative TLC plot and quantification of WT vs. RAG KO fur lipids (n=5 mice/group). (FIG. 41H) Representative TLC plot of WT vs. E-Beta KO fur lipids. Data are shown as mean s.e.m. from 2 experiments. Student's t-test. Statistics. (FIG. 41C) t=0.9520, df=199. (FIG. 41E) t=3.3460, df=276. (FIG. 41G) CE: t=1.520, WE: t=3.924, FFA: t=1.748, FC: t=1.052, df=20 for all comparisons.

FIGS. 42A-42R illustrate TSLP expression is positively correlated with the expression of multiple sebaceous gland genes in healthy human skin. Linear regression analysis of TSLP expression vs. expression of a panel of SG-associated genes: (FIG. 42A) SCD, (FIG. 42B) FADS2, (FIG. 42C) PPARg, (FIG. 42D) FA2H, (FIG. 42E) DGAT1, (FIG. 42F) DGAT2, (FIG. 42G) FABP4, (FIG. 42H) FABP5, (FIG. 42I) ACACA, (FIG. 42J) FASN, (FIG. 42K) AWAT1, (FIG. 42L) ELOVL1, (FIG. 42M) ELOVL3, (FIG. 42N) ELOVL4, (FIG. 42O) ELOVL5, (FIG. 42P) MOGAT1, (FIG. 42Q) MOGAT2, (FIG. 42R) MOGAT3 (n=36 healthy individuals). Pearson's r and Linear regression slope test. Statistics. (FIG. 42A) F=5.691, (FIG. 42B) F=5.09, (FIG. 42C) F=1.202, (FIG. 42D) F=6.386, (FIG. 42E) F=0.9058, (FIG. 42F) F=8.930, (FIG. 42G) F=0.0543, (FIG. 42H) F=6.016, (FIG. 42I) F=4.342, (FIG. 42J) F=4.138, (FIG. 42K) F=5.363, (FIG. 42L) F=6.801, (FIG. 42M) F=9.739, (FIG. 42N) F=3.467, (FIG. 42O) F=3.903, (FIG. 42P) F=3.156, (FIG. 42Q) F=3.872, (FIG. 42R) F=0.0108, dfₙ=1 and df_{d}=34 for all comparisons.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “antibody,” as used herein, refers to an immunoglobulin molecule that specifically binds with an antigen. Antibodies can be intact immunoglobulins derived from natural sources or from recombinant sources, and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, single-domain antibodies such as sdAb (either VL or VH), such as camelid antibodies (Riechmann, 1999, J. Immunol. Meth. 231:25-38), camelid VHH domains, composed of either a VL or a VH domain that exhibit sufficient affinity for the target, and multispecific antibodies formed from antibody fragments such as a bivalent fragment comprising two Fab fragments linked by a disulfide bridge at the hinge region, and an isolated complementarity-determining region (CDR) or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger & Hudson, 2005, Nature Biotech. 23:1126-1136). Antigen binding fragments can also be grafted into scaffolds based on polypeptides such as a fibronectin type III (Fn3) (U.S. Pat. No. 6,703,199, which describes fibronectin polypeptide minibodies). The antibody fragment also includes a human antibody or a humanized antibody or a portion of a human antibody or a humanized antibody.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “HFD” refers to high fat diet.

As used herein, the term “NC” refers to normal chow, which is a normal diet.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

As used herein, the term “TSLP” refers to thymic stromal lymphopoietin.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Preparation of Compounds

The compounds described herein can be prepared by the general schemes described herein, using the synthetic method known by those skilled in the art. The following examples illustrate non-limiting embodiments of the compound(s) described herein and their preparation.

The compounds described herein can possess one or more stereocenters, and each stereocenter can exist independently in either the (R) or (5) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³K, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

Compounds described herein are synthesized using any suitable procedures starting from compounds that are available from commercial sources, or are prepared using procedures described herein.

### Compositions

In various embodiments, compositions useful for the methods described herein include at least one vitamin D₃ analog. In certain embodiments, the vitamin D₃ analog is at least one analog selected from the group consisting of:
26,27-cyclo-22-ene-1α,24S-dihydroxyvitamin D₃ (also known as MC903, calcipotriene, or calcipotriol);
1α,18,25-(OH)₃D₃,
23-(m-(Dimethylhydroxymethyl)-22-yne-24,25,26,27(tetranor)-1α-OH)₂D₃;
1α,25-Dihydroxy-trans-Isotachysterol (also known as 1,25-trans-Iso-T);
(1S,3R,6S)-7,19-Retro-1,25-(OH)₂D₃;
(1S,3R,6R)-7,19-Retro-1,25-(OH)₂D₃;
22-(p-(Hydroxyphenyl)-23,24,25,26,27-pentanor-D₃;
22-(m-(Hydroxyphenyl)-23,24,25,26,27-pentanor-D₃;
1(S),3(R)-dihydroxy-20(R)-(5′-ethyl-5′-hydroxy-hepta-1′(E),3′(E)-dien-1′-yl)-9,10-secopregna-5(Z),7(E),10(19)-triene (also known as EB1089);
1α,25-(OH)-20-epi-22-oxa-24,26,27-trishomovitamin D (also known as KH1060);
22-oxa-1α,25(OH)₂D₃ (also known as OCT or 22-OXA);
1R,25-dihydroxy-21-(3-hydroxy-3-methylbutyl) vitamin D₃;
   and any combinations thereof.

In certain embodiments, the vitamin D₃ analog is MC903. In certain embodiments, the vitamin D₃ analog is the only biologically active agent administered to the subject. In other embodiments, MC903 is the only biologically active agent administered to the subject. In yet other embodiments, the analog is the only biologically active agent administered to the subject in an amount sufficient to increase systemic TSLP levels in the subject. In yet other embodiments, MC903 is the only biologically active agent administered to the subject in an amount sufficient to increase systemic TSLP levels in the subject.

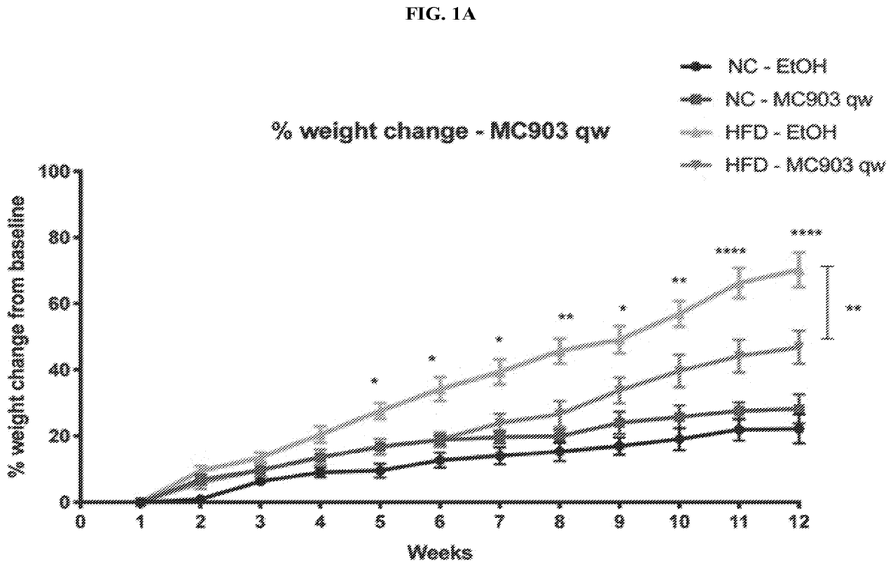

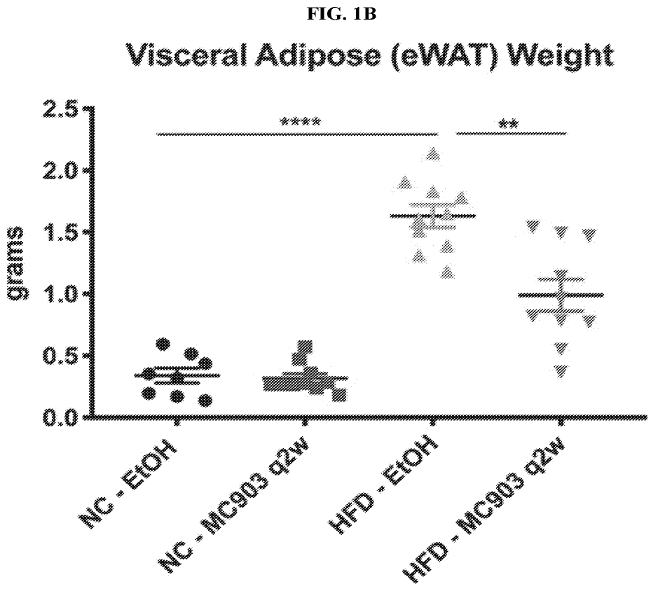

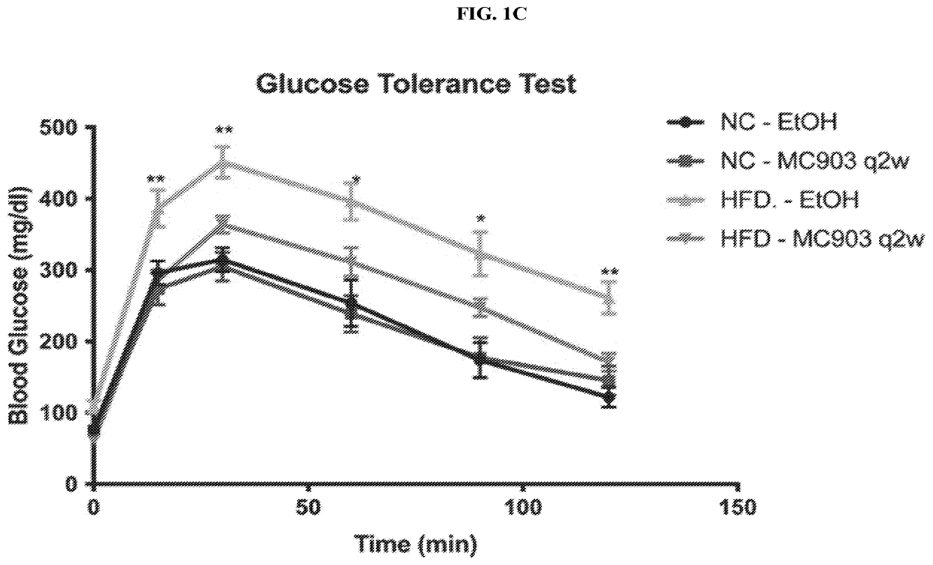

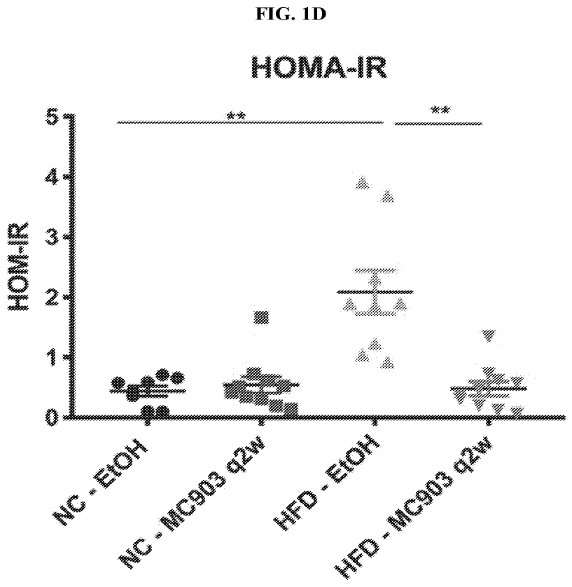

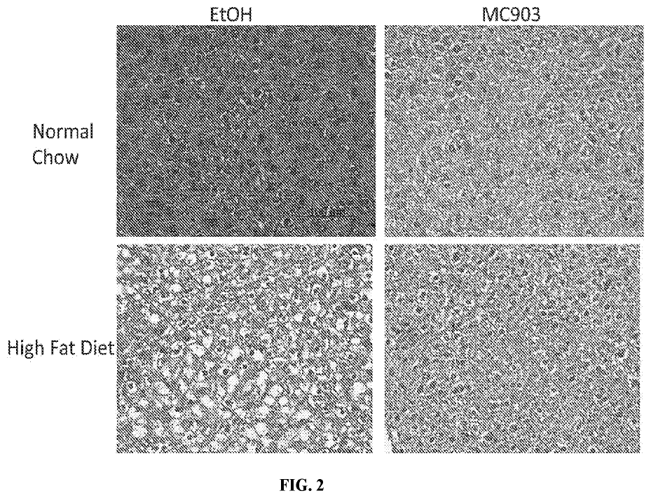

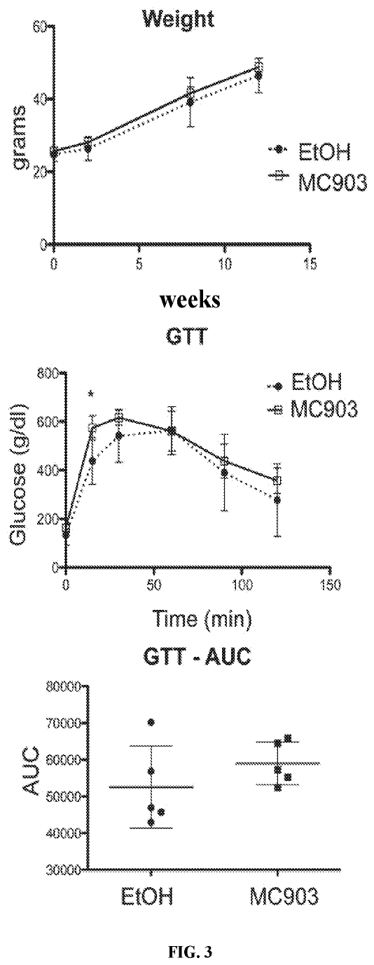

TSLP levels can be elevated in subjects by inducing its production by the topical administration of MC903. Compared to vehicle (EtOH), the topical administration of MC903 over 12 weeks leads to significantly reduced weight gain of mice that have been fed a high fat diet (HFD) (FIG. 1A). The decreased weight gain is associated with significantly decreased epididymal white adipose tissue (eWAT) weight and improved metabolic parameters, as judged by glucose tolerance test (GTT) and homeostatic model assessment of insulin resistance (HOMA-IR) (FIGS. 1B-1D). Fat deposition in the liver is also markedly reduced (FIG. 2) in mice treated with MC903. The loss of eWAT, improved GTT and HOMA-IR values, and reduced hepatic liver deposition resulting from topical MC903 administration is dependent on the presence of TSLP in the body, since MC903 treatment does not prevent weight gain or improve metabolic parameters in TSLP-R-deficient mice (FIG. 3).

### Compositions of TSLP

In various embodiments, a composition useful for the methods described herein includes a TSLP polypeptide isoform or a viral vector that includes a TSLP-expression sequence.

### TSLP Isoforms

In various embodiments, the subject is human and the TSLP isoform has at least 85% identity, at least 90% identity, at least 95% identity, or at least 99% identity with at least one polypeptide of SEQ ID NO:1 (MFPFALLYVL SVSFRKIFIL QLVGLVLTYD FTNCDFEKIK AAYLSTISKD LITYMSGTKS TEFNNTVSCS NRPHCLTEIQ SLTFNPTAGC ASLAKEMFAM KTKAALAIWC PGYSETQINA TQAMKKRRKR KVTTNKCLEQ VSQLQGLWRR FNRPLLKQQ) or SEQ ID NO:2 (MFAMKTKAAL AIWCPGYSET QINATQAMKK RRKRKVTTNK CLEQVSQLQG LWRRFNRPLL KQQ). In other embodiments, the subject is human and the TSLP isoform is at least one selected from the group consisting of SEQ ID NO:1 and SEQ ID NO:2.

The TSLP isoforms can be stabilized for in vitro and/or in vivo administration of the peptide to provide a stabilized form that is suitable for a particular route of administration, such as through intravenous, subcutaneous, or other intraperitoneal administration routes. Stabilized TSLP isoforms of SEQ ID NO:1, SEQ ID NO:2, and peptides having at least 90% identity, at least 95% identity, or at least 99% identity to SEQ ID NO:1 or SEQ ID NO:2 include peptides having substitution of one or more residues in the primary peptide sequence. The substitution can include changing the one or more amino acids into another homologous naturally occurring amino acid, a D-form of an amino acid, a synthetic derivative of an amino acid, or a peptidomimetic moiety that mimics the physicochemical properties of an amino acid. Stabilized TSLP isoforms can also include addition of cysteine residues or replacement of non-cysteine residues with cysteine so that the TSLP isoform can form disulfide bonds in solution, both in vitro and in vivo. Stabilized TSLP isoforms can also include addition of chemical moieties at the N- or C-terminal residues, or both, such as by the addition of one or more polyethylene glycol (PEG) moieties. Suitable PEG moieties can contain from about to about 100 ethylene glycol units. The PEG moieties can be covalently attached to the peptide by any suitable chemical means, including via amide bonds, C₁-C₁₀ alkyl linkers, carbamates, carbonates, and the like. In various embodiments, stabilized TSLP isoforms of SEQ ID NO:1 or SEQ ID NO:2, and peptides having at least 90% identity, at least 95% identity, or at least 99% identity to SEQ ID NO:1 or SEQ ID NO:2 retain at least about 50%, 55%, 60%, 65%, 70%, 75%, 80%, 85%, 90%, 95%, 99%, or 99.5% of the activity of an unmodified/unstabilized peptide.

### TSLP-Containing Viral Vectors

In various embodiments, TSLP expression can be enhanced in vitro or in vivo with a viral vector that incorporates a TSLP-expression sequence and at least one promoter. The viral vector can be any suitable adeno-associated virus (AAV), such as the AAV1-AAV8 family of adeno-associated viruses. In some embodiments, the viral vector is a viral vector that can infect a human. The TSLP-expression sequence can be inserted between the inverted terminal repeats (ITRs) in the AAV. The TSLP-expression sequence can be any suitable mammalian TSLP sequence. Non-limiting examples of suitable mammalian TSLP-expression sequences include mouse, human, pig, dog, cow, horse, cat, or horse TSLP-expression sequences. In certain embodiments, the TSLP-expression sequence is a human sequence. In various embodiments, the viral vector is an AAV8. The promoter can be a thyroxine binding globulin (TBG) promoter. In various embodiments, the promoter is a human promoter sequence that enables TSLP-expression in the liver. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. Suitable AAV's that express human or murine TSLP with a TBG promoter can be purchased from Vector Biolabs (Malvern, Pa.). In various embodiments, TSLP expression can be enhanced in vitro or in vivo with an AAV8-TSLP vector. As used herein, the term “AAV8-TSLP” means an AAV8 viral vector (recombinant or non-recombinant) containing a TSLP-expression sequence and at least one promoter sequence that, when administered to a subject, causes elevated systemic expression of TSLP. In some embodiments, the viral vector is a recombinant or non-recombinant AAV2 or AAV5 containing any of the TSLP-expression sequences described herein.

The TSLP-expression sequence can, in some embodiments, be a long form mammalian TSLP-expression sequence or a short form mammalian TSLP-expression sequence. In some embodiments, the TSLP-expression sequence is a long form human TSLP-expression sequence of SEQ ID NO:3:

```
a tgttcccttt tgccttacta tatgttctgt cagtttcttt
    caggaaaatc ttcatcttac aacttgtagg gctggtgtta acttacgact tcactaactg
    tgactttgag aagattaaag cagcctatct cagtactatt tctaaagacc tgattacata
    tatgagtggg accaaaagta ccgagttcaa caacaccgtc tcttgtagca atcggccaca
    ttgccttact gaaatccaga gcctaacctt caatcccacc gccggctgcg cgtcgctcgc
    caaagaaatg ttcgccatga aaactaaggc tgccttagct atctggtgcc caggctattc
    ggaaactcag ataaatgcta ctcaggcaat gaagaagagg agaaaaagga aagtcacaac
    caataaatgt ctggaacaag tgtcacaatt acaaggattg tggcgtcgct tcaatcgacc
    tttactgaaa caacagtaa.
```

In various embodiments, the TSLP-expression sequence is a long form human TSLP-expression sequence of SEQ ID NO:4:

MFPFALLYVLSVSFRKIFILQLVGLVLTYDFINCDFEKIKAAYLSTISKDLITYMSGTKSTEFNNTVS
CSNRPHCLTEIQSLTFNPTAGCASLAKEMFAMKTKAALAIWCPGYSETQINATQAMKKRRKRKVTTNK
CLEQVSQLQGLWRRFNRPLLKQQ.

In various embodiments, the TSLP-expression sequence is short form human TSLP-expression sequence of SEQ ID NO:5:

```
a tgttcgccat gaaaactaag gctgccttag ctatctggtg cccaggctat
    tcggaaactc agataaatgc tactcaggca atgaagaaga ggagaaaaag
    gaaagtcaca accaataaat gtctggaaca agtgtcacaa ttacaaggat
    tgtggcgtcg cttcaatcga cctttactga aacaacagta a.
```

In various embodiments, the TSLP-expression sequence is a short form human TSLP-expression sequence of SEQ ID NO:6:

MFAMKTKAALAIWCPGYSETQINATQAMKKRRKRKVTTNKCLEQVSQLQGLWRRFNRPLLKQQ.

In various embodiments, the TSLP-expression sequence is a long form mouse TSLP-expression sequence of SEQ ID NO:7:

atg gttcttctca ggagcctctt catcctgcaa gtactagtac
61 ggatggggct aacttacaac ttttctaact gcaacttcac gtcaattacg aaaatatatt
121 gtaacataat ttttcatgac ctgactggag atttgaaagg ggctaagttc gagcaaatcg
181 aggactgtga gagcaagcca gcttgtctcc tgaaaatcga gtactatact ctcaatccta
241 tccctggctg cccttcactc cccgacaaaa catttgcccg gagaacaaga gaagccctca
301 atgaccactg cccaggctac cctgaaactg agagaaatga cggtactcag gaaatggcac
361 aagaagtcca aaacatctgc ctgaatcaaa cctcacaaat tctaagattg tggtattcct
421 tcatgcaatc tccagaataa.

In various embodiments, the TSLP-expression sequence is a long form mouse TSLP-expression sequence of SEQ ID NO:8:

MVLLRSLFILQVLVRMGLTYNFSNCNFTSITKIYCNIIFHDLTGDLKGAKFEQIEDCESKPACLIKIE
YYTLNPIPGCPSLPDKTFARRTREALNDHCPGYPETERNDGTQEMAQEVQNICLNQTSQILRLWYSFM
QSPE.

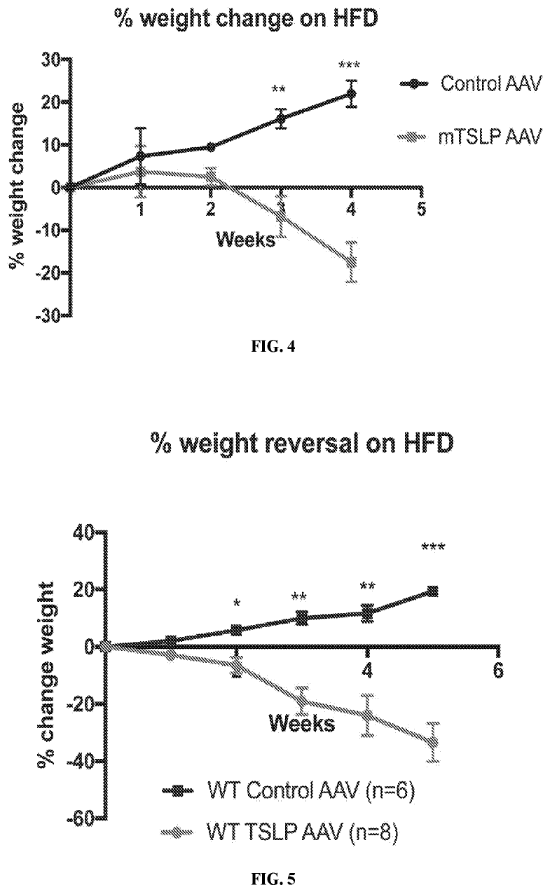

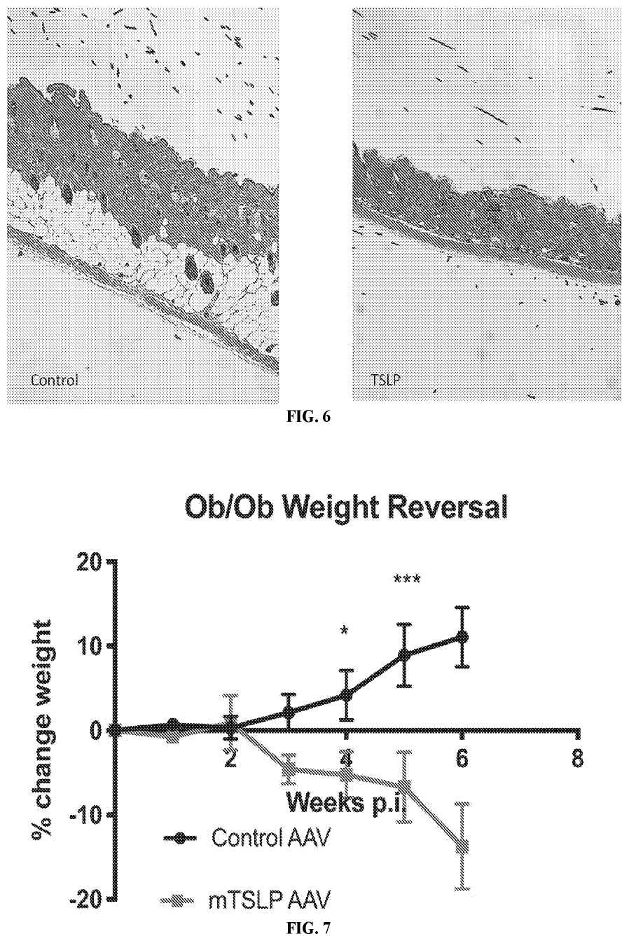

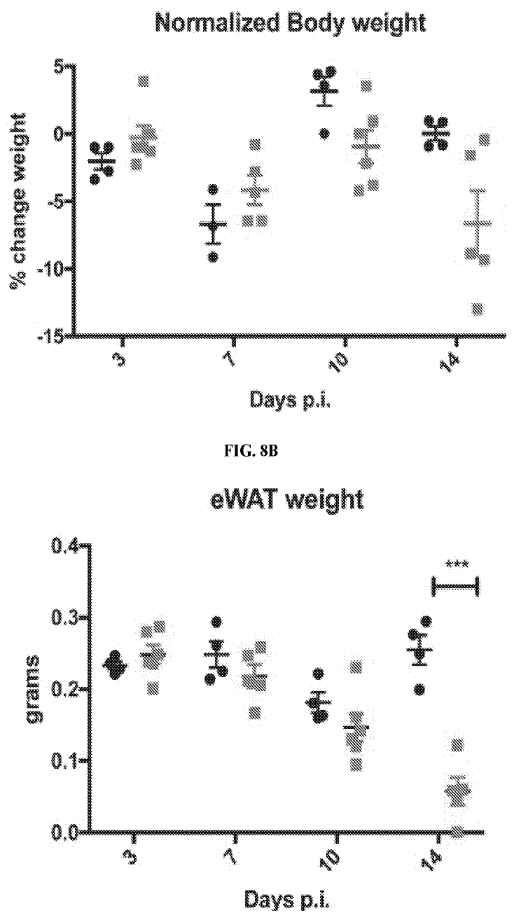

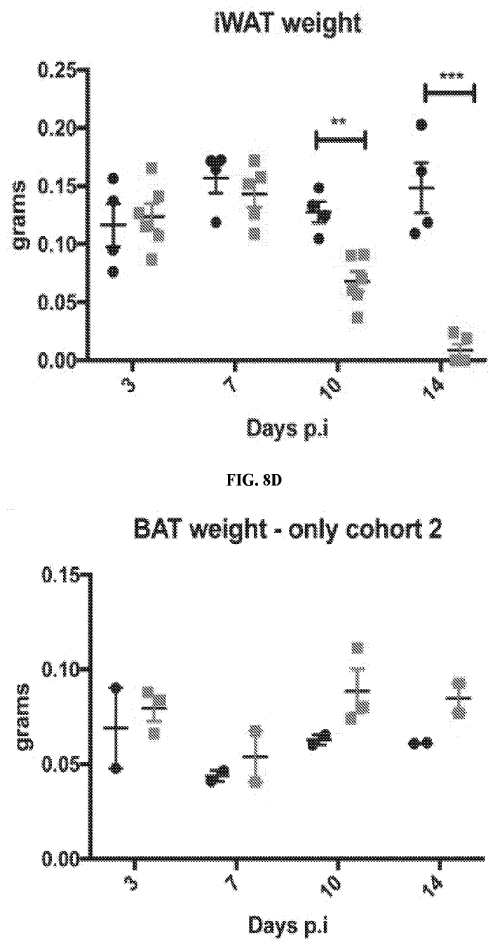

To test whether TSLP alone was sufficient in preventing weight gain in HFD-fed mice, an adeno-associated virus expressing TSLP (AAV8-TSLP) was injected into mice. The injection of AAV8-TSLP drives expression of TSLP over 6-8 weeks in the liver using a thyroxine binding globulin (TBG) promoter. Compared to AAV8-control, mice that received AAV8-TSLP lost weight despite being on a HFD (FIG. 4). Moreover, AAV8-TSLP injection caused significant weight loss in mice that were already overweight (previously on HFD for 10 weeks), despite continuing on a HFD (FIG. 5). At the end of 5 weeks, skin histology revealed the complete loss of subcutaneous fat from these mice (FIG. 6). The effect of TSLP was also tested on Ob/Ob mice. Ob/Ob mice are deficient in leptin, which results in hyperphagia (too much eating) and consequent weight gain, despite being fed normal chow. Similar to HFD-fed mice, weight loss was also observed when Ob/Ob mice were treated with AAV8-TSLP (FIG. 7). AAV8-TSLP also induced adipose tissue loss in normal chow-fed mice. Although only slight weight loss was observed in normal chow-fed mice treated with AAV8-TSLP (FIG. 8A), a near complete loss of the eWAT and inguinal white adipose tissue (iWAT) weights were observed between Day 7 and Day 14 post injection of AAV8-TSLP compared to AAV8-control (FIGS. 8B, 8C). This effect was specific to white adipose tissue, since no differences in brown adipose tissue (BAT) or muscle weight were seen in AAV8-TSLP compared to AAV8-control-injected mice (FIGS. 8D, 8E).

The compositions containing the compound(s), peptides, and/or viral vectors described herein include a pharmaceutical composition comprising at least one compound, peptide, and/or viral vector as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Methods of Treating, Ameliorating, and/or Preventing Obesity and/or Skin Disorders by Elevating TSLP Levels

### Obesity:

MC903

In various embodiments, methods of treating obesity or obesity-related disorders are provided. The method includes topically administering at least one vitamin D₃ analog, as described herein, to a subject suffering from obesity or an obesity-related disorder. The types of obesity-related disorders that can be treated with these methods is not particularly limited, and any disorder that results from a subject having excess white adipose tissue in the body can be treated with the compositions and methods described herein. Non-limiting examples of obesity-related disorders include nonalcoholic steatohepatitis (NASH), metabolic diseases, diabetes (type I and type II), hypertension, dyslipidemia (high LDL cholesterol, low HDL cholesterol, or high levels of triglycerides), coronary heart disease, stroke, gallbladder disease, kidney disease, osteoarthritis, sleep apnea and breathing problems, and cancers, including endometrial, breast, colon, kidney, gallbladder, and liver cancer. In various embodiments, the obesity-related disorder is NASH.

Obesity can be determined by a variety of art-recognized methods, including measuring a subject's body mass index (BMI), waist circumference, waist-to-hip ratio, skinfold thickness, dual energy X-ray absorptiometry (DEXA), blood triglyceride levels, blood cholesterol levels, and the like. The standards for determining whether a human subject is obese or not include the standards promulgated by the U.S. Centers for Disease Control and Prevention (CDC). In various embodiments, an obese human subject has a BMI of at least about 25.0, 30.0, 35.0, or 40.0. In general, human subjects with a BMI of less than about 25.0 are not considered obese, however the compositions described herein can also be used by non-obese individuals to reduce the amount of white adipose tissue in their bodies for aesthetic, cosmetic, athletic, or other purposes.

In various embodiments, a method of treating obesity or an obesity-related disorder results in weight loss in the subject. The weight loss resulting from the applying the methods described herein can result in at least, great than, or less than about 5%, 7.5%, 10%, 12.5%, 15%, 17.5%, or a 20% reduction in weight over a period of about 1, 2, 3, 4, 5, 6, 7, 8, 9, 10, 11, or 12 weeks. As shown in FIG. 1A, mice on a high fat diet (HFD) that had MC903 administered to them experienced significantly less weight gain over a 12 week period as compared to control mice that were not administered MC903. FIG. 2 shows that HFD control mice (no MC903 administered) developed significant amounts of white adipose tissue (bottom left image), whereas HFD mice that had MC903 administered to them had white adipose tissue distribution that appeared very similar to the tissue of mice fed with normal chow. Administration of MC903, in various embodiments, results in substantially no loss of muscle mass in a subject. In various embodiments, the administering causes secretion of lipids from the subject's skin. In certain embodiments, the loss of white adipose tissue from the subject's body results in secretion of the lipid components of the white adipose tissue from the subject's skin.

TSLP Peptide and TSLP-Containing Viral Vectors

In various embodiments, methods of treating obesity or obesity-related disorders are provided. The method includes administering at least one TSLP polypeptide isoform or at least one viral vector that includes a TSLP-expression sequence, as described herein, to a subject suffering from obesity or an obesity-related disorder.

The types of obesity-related disorders that can be treated with these methods is not particularly limited, and any disorder that results from a subject having excess white adipose tissue in the body can be treated with the compositions and methods described herein. Non-limiting examples of obesity-related disorders include nonalcoholic steatohepatitis (NASH), metabolic diseases, diabetes (type I and type II), hypertension, dyslipidemia (high LDL cholesterol, low HDL cholesterol, or high levels of triglycerides), coronary heart disease, stroke, gallbladder disease, kidney disease, osteoarthritis, sleep apnea and breathing problems, and cancers, including endometrial, breast, colon, kidney, gallbladder, and liver cancer. In various embodiments, the obesity-related disorder is NASH.

In various embodiments, a method of treating obesity or an obesity-related disorder results in weight loss in the subject. The weight loss resulting from administering at least one TSLP polypeptide isoform or at least one viral vector that includes a TSLP-expression sequence using the methods described herein can result in at least, great than, or less than about 5%, 7.5%, 10%, 12.5%, 15%, 17.5%, or a 20% reduction in weight over a period of about 1, 2, 3, 4, 5, 6, 7, 8, 9, 10, 11, or 12 weeks.

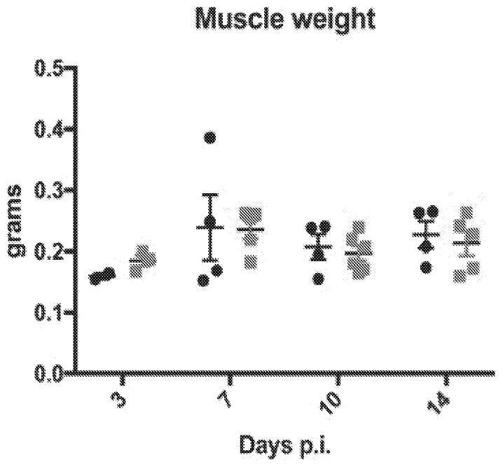

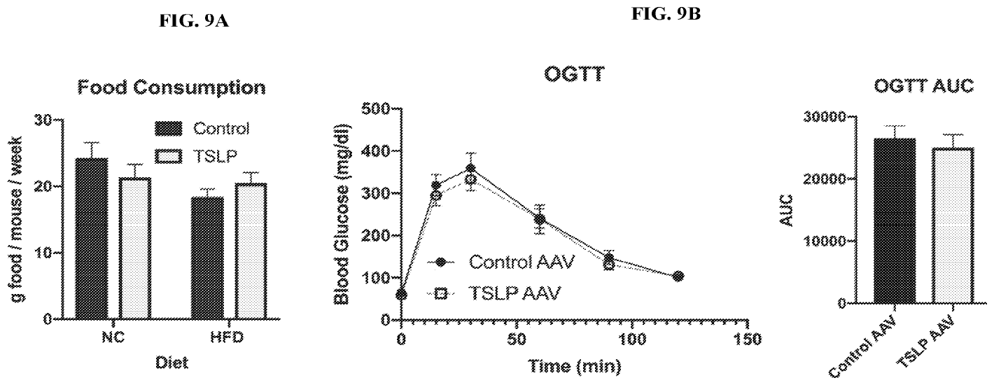

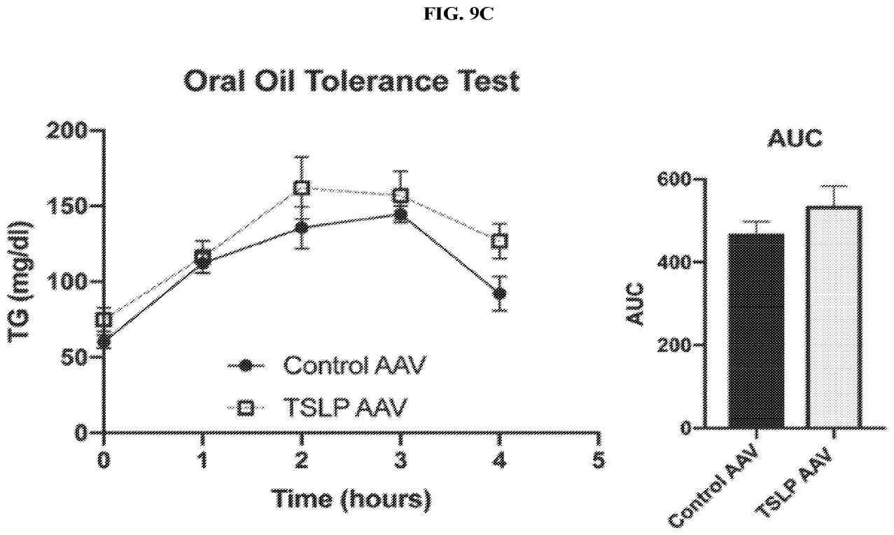

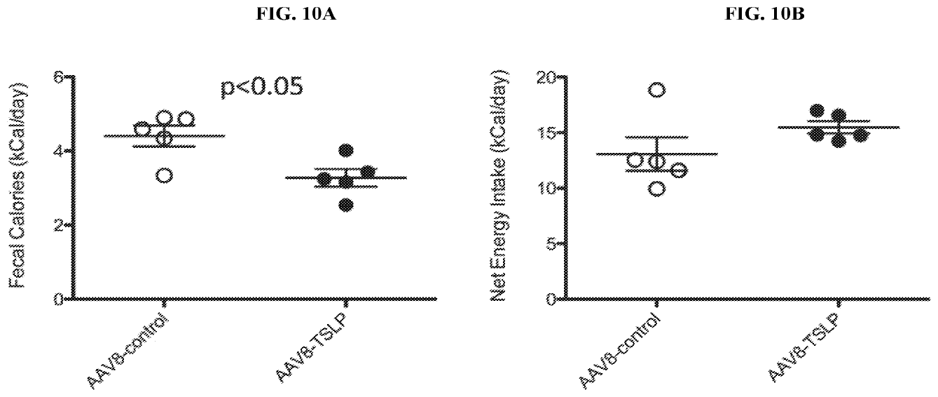

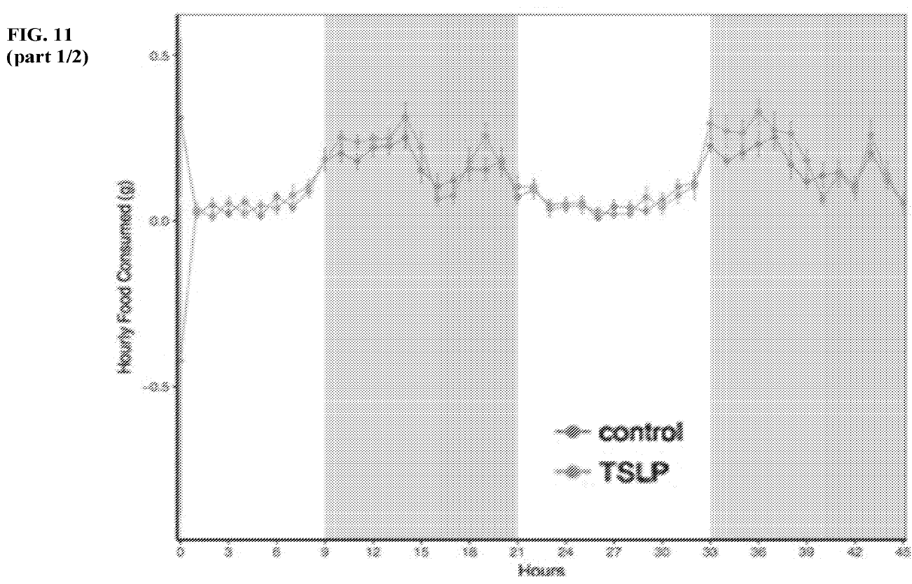

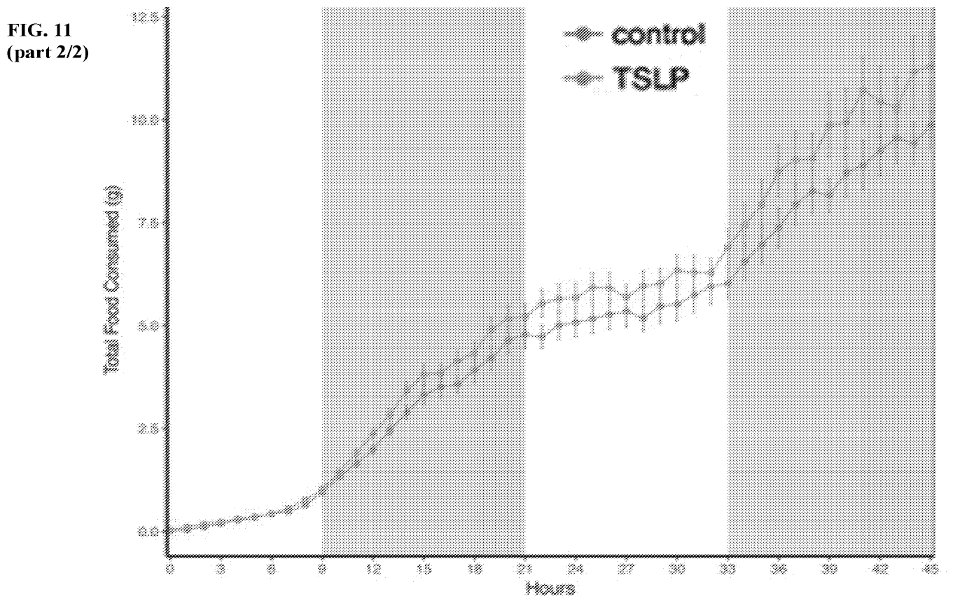

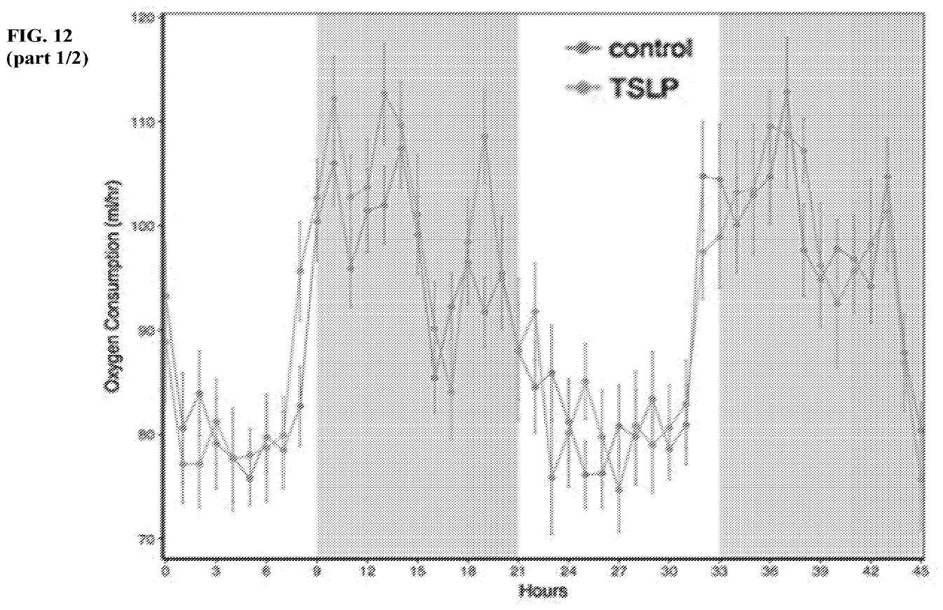

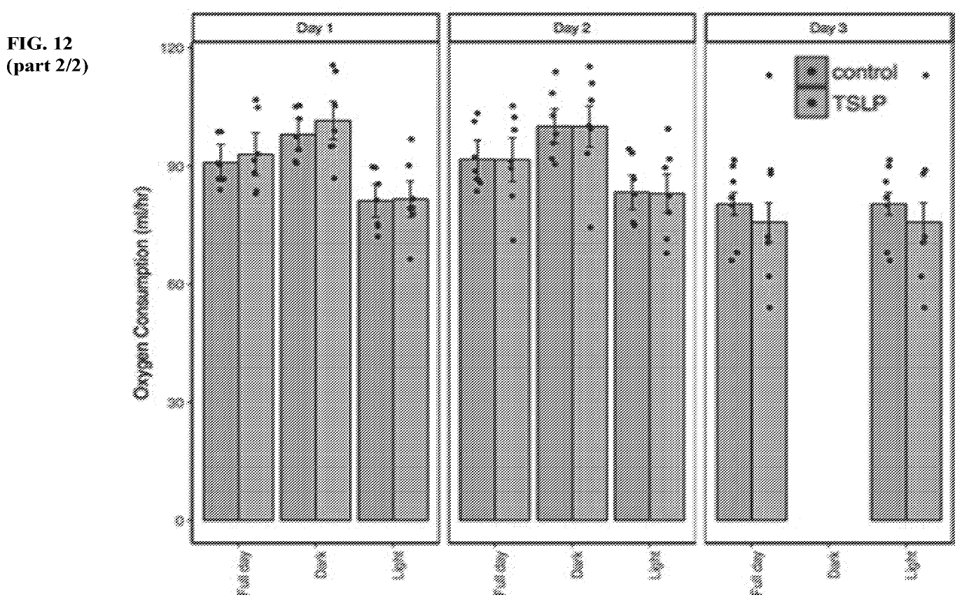

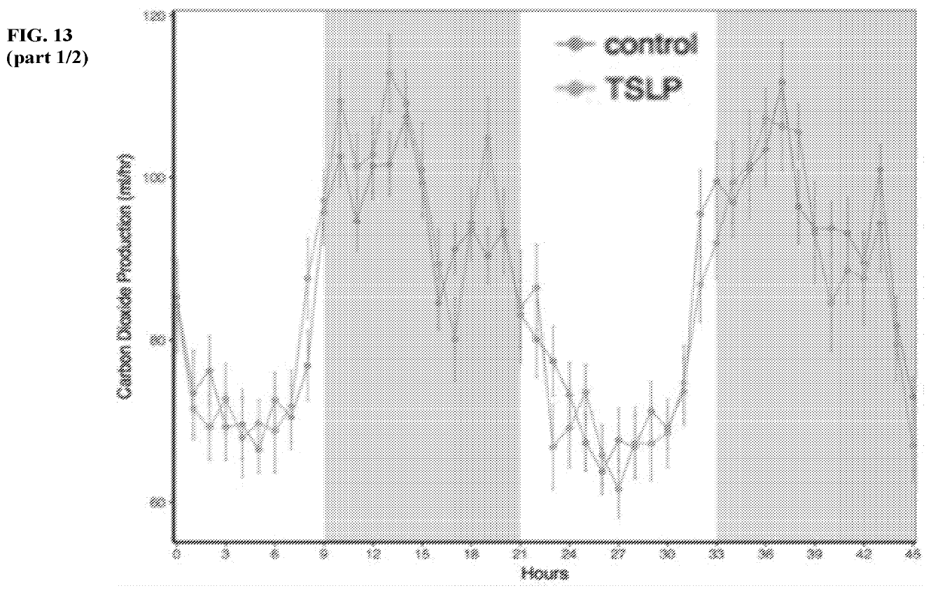

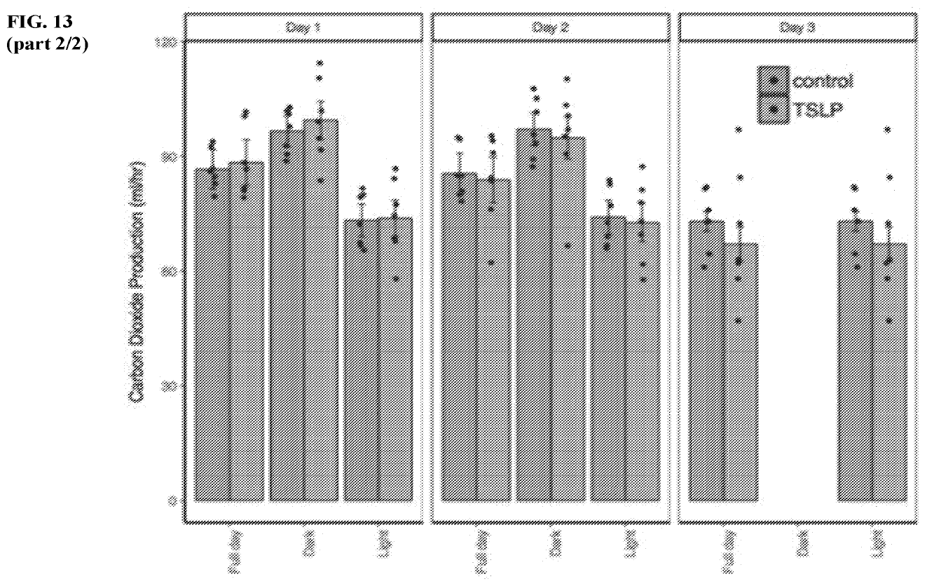

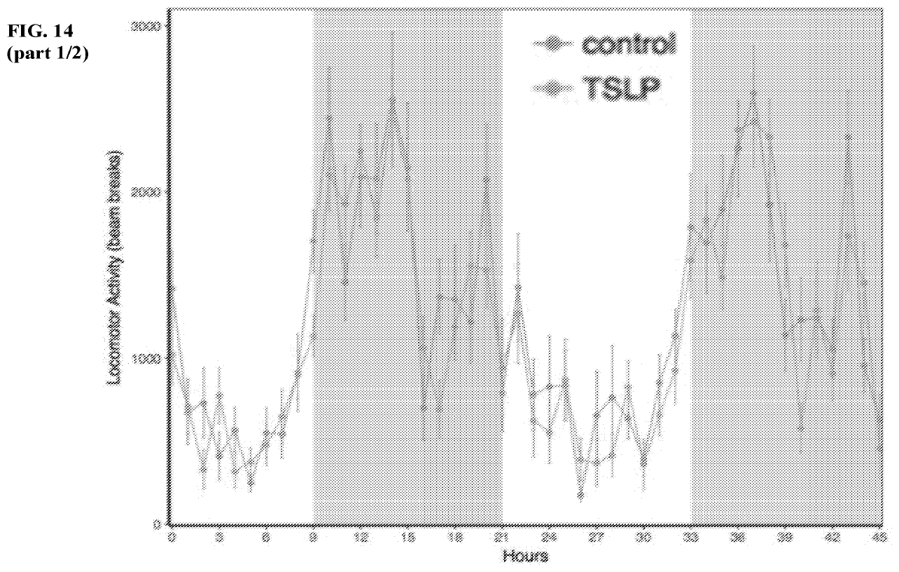

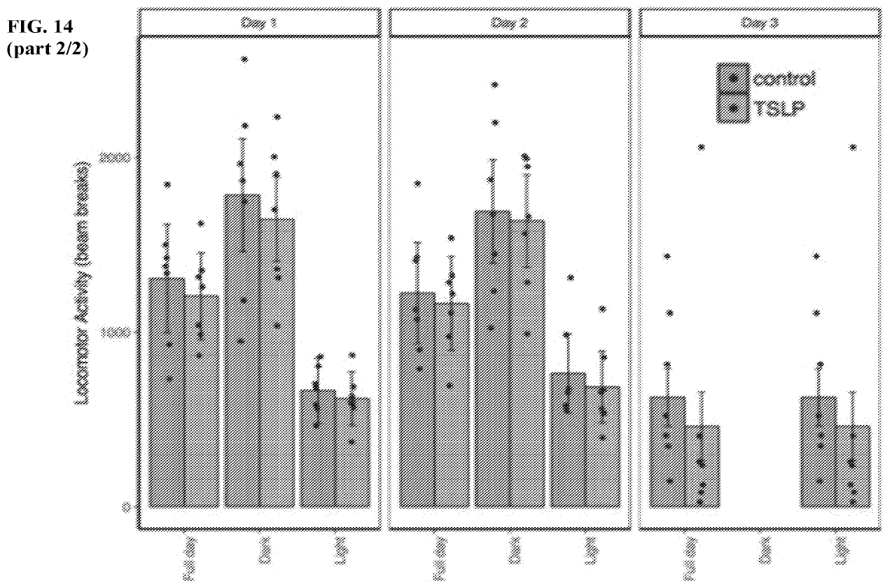

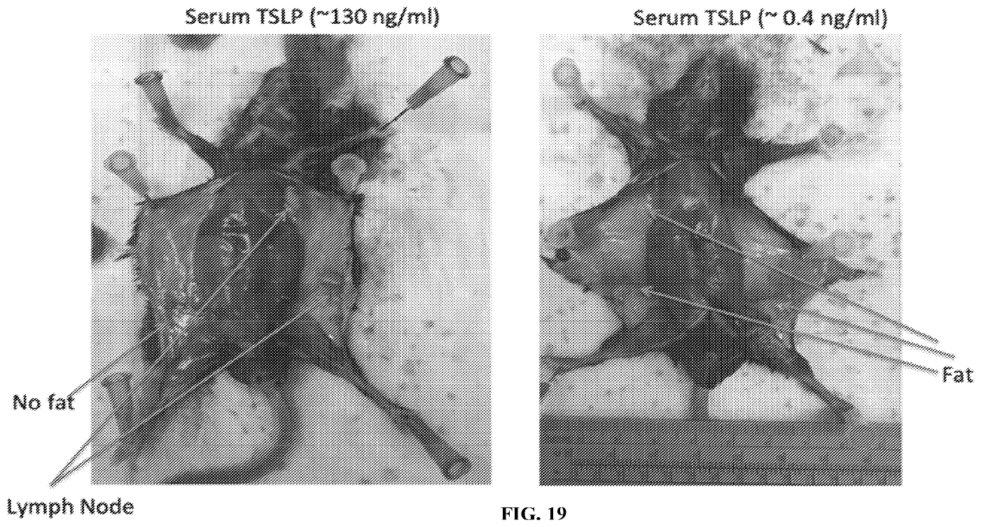

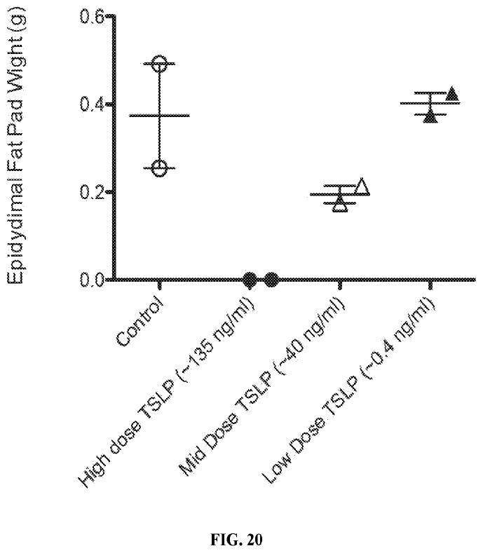

In order for a subject to lose weight, a net negative energy imbalance must exist, i.e., there is a decrease in energy intake (food intake/absorption), increase in energy expenditure (heat generation, increased locomotor activity), and/or increased energy output (excretion or secretion outside of the body). Energy intake by AAV8-control vs. AAV8-TSLP was similar based on food consumption (FIG. 9A), glucose absorption (FIG. 9B), and lipid absorption (FIG. 9C). In addition, bomb calorimetry of a 1-day stool collection from AAV8-control vs. AAV8-TSLP-treated mice revealed significantly less energy in the stool, translating to a trend towards a net increase in energy intake (FIG. 10). To more accurately measure energy intake and expenditure, AAV-control and AAV8-TSLP-treated mice were placed in a metabolic chamber from Day 7 to Day 10 post AAV8 injection. According to these accurate measurements, food intake was in fact increased in the AAV8-TSLP-treated mice by ˜20% over the last 48 hour period in the metabolic chamber (FIG. 11). Despite losing 30% of their fat mass during the 72-hour period that the mice were placed in a metabolic chamber, O₂ consumption and CO₂ production (both measures of energy expenditure), and locomotor activity were all similar between AAV8-control and AAV8-TSLP-treated mice (FIGS. 12-14). Together, these data suggest, without being bound by theory, that TSLP does not induce white adipose loss by decreasing energy uptake or increasing energy expenditure. The weight loss in mice is further illustrated in FIG. 19 and FIG. 20.

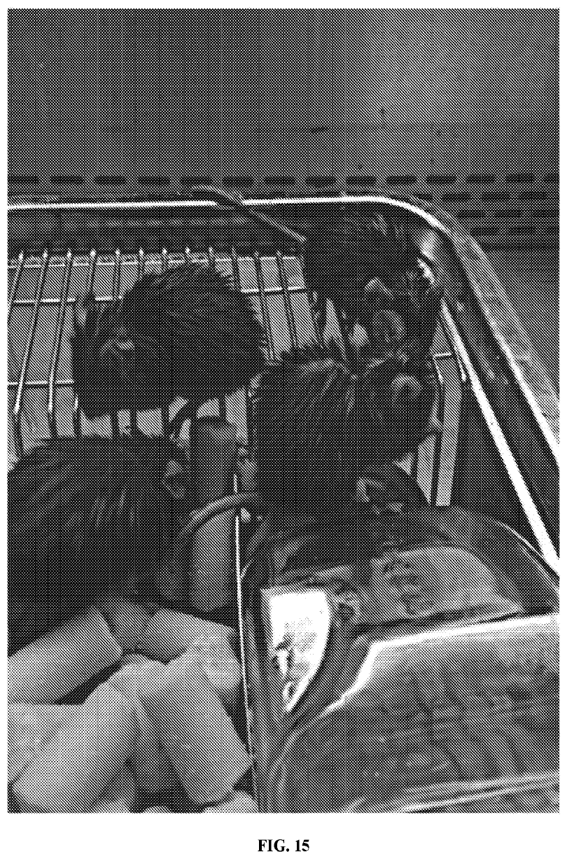

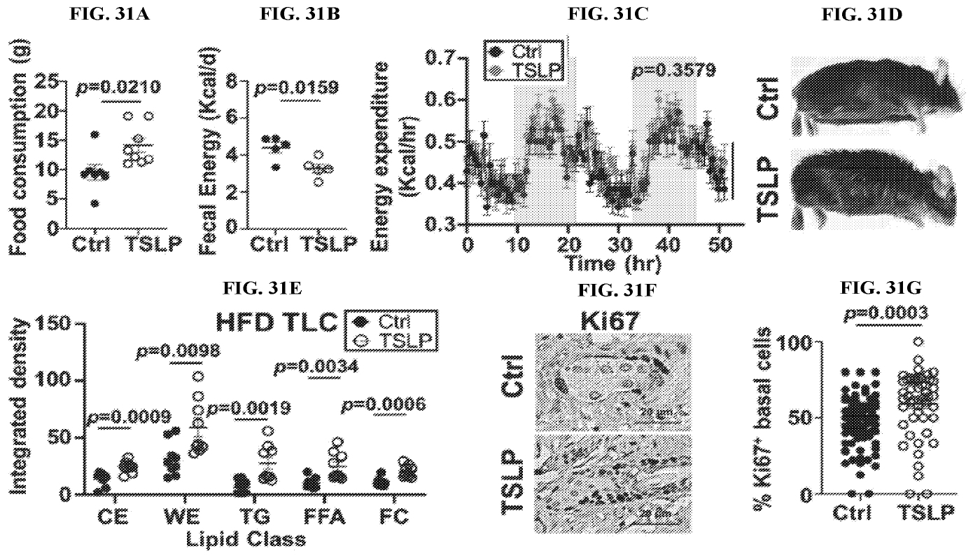

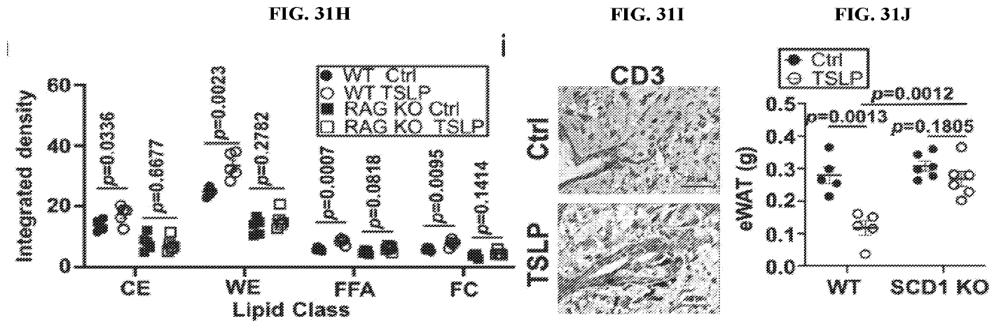

Although not apparent when mice are placed on normal chow until about week 4, obese mice that are treated with AAV8-TSLP develop very oily fur as the mice lose weight and their fat mass (FIG. 15). This sebum secretion is responsible for adipose tissue loss, since stearoyl-CoA desaturase 1 (SCD1) knockout mice, which have defective sebaceous glands do not lose adipose tissue upon AAV8-TSLP treatment (FIG. 31J).

Thus, without wishing to be bound by theory, TSLP causes adipose loss by inducing the secretion of lipids (e.g., through sebum) through the skin. In various embodiments, the administering causes secretion of lipids from the subject's skin. In certain embodiments, the loss of white adipose tissue from the subject's body results in secretion of the lipid components of the white adipose tissue from the subject's skin.

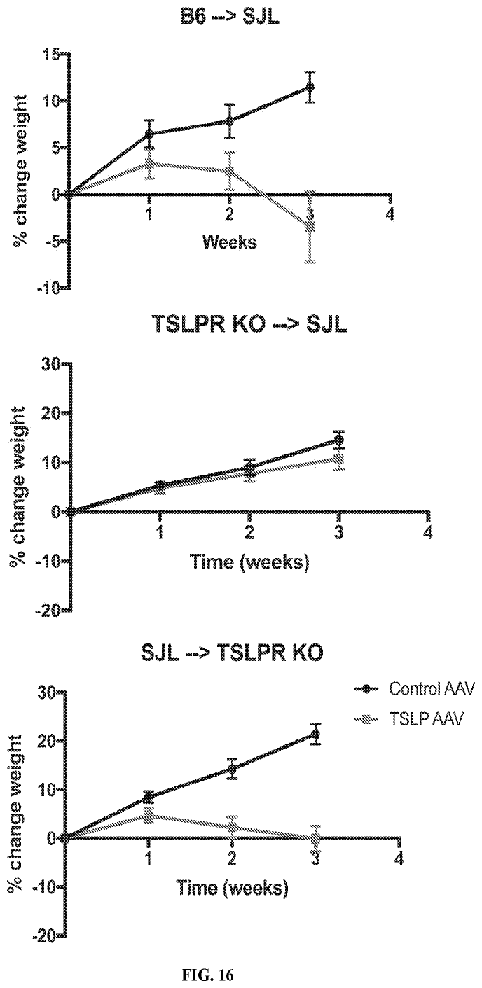

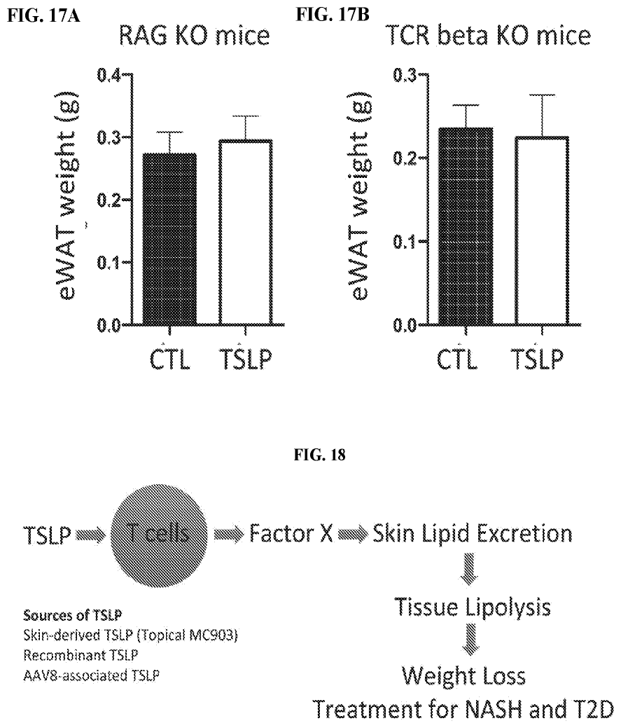

To test whether TSLP responsiveness was necessary in the hematopoietic or non-hematopoietic compartment, bone marrow (BM) transplantation studies were performed. BM transplantation allows one to replace the hematopoietic compartment with BM from another mouse. Thus, B6.SJL mice transplanted with TSLP-R KO BM (TSLP-R KO→B6.SJL) responds to TSLP in the non-hematopoietic but not hematopoietic compartment. Conversely, TSLP-R KO mice transplanted with B6.SJL BM (B6.SJL→TSLP-R KO) responds to TSLP in the hematopoietic but not non-hematopoietic compartment. When treated with AAV8-TSLP, while B6.SJL→B6 and B6.SJL→TSLP-R KO mice lost weight, TSLP-R KO→B6.SJL mice failed to lose weight, suggesting that TSLP signaling must occur in the hematopoietic compartment (FIG. 16). To more precisely identify the cell type involved, RAG KO (mouse with no T or B cells) and TCR-beta KO mice (no T cells) were treated with AAV8-TSLP. Both RAG KO mice and TCR-beta KO mice failed to lose weight when treated with AAV8-TSLP, suggesting that T cells are required for the TSLP-induced weight loss (FIG. 17). Without being bound by theory, TSLP-stimulated T cells can produce a factor that induces lipid secretion from the skin, which drives weight loss in these mice and whereby TSLP causes the selective loss of white adipose from mice.

The methods described herein increase TSLP levels in the subject. In various embodiments, the TSLP levels in the body of a subject are increased by at least, greater than, or less than about 5% to about 40%, about 5% to about 30%, about 5% to about 20%, or about 5% to about 10% relative to a control. In various embodiments, the TSLP levels in the body of a subject are increased by at least, greater than, or less than about 5%, 10%, 15%, 20%, 25%, 30%, 35%, or about 40% relative to a control. Administration of at least one TSLP polypeptide isoform or at least one viral vector that includes a TSLP-expression sequence, in various embodiments, results in substantially no loss of muscle mass in a subject.

TSLP can be elevated in mice through topical MC903 treatment, recombinant TSLP polypeptide isoforms, or through viral vectors (gene therapy). Elevation of TSLP causes skin secretion of lipids, which consumes energy and decreases circulating lipid levels. Lipolysis occurs from fat stores in order to replenish the consumed lipids, which eventually leads to selective depletion of white adipose tissue and weight loss (FIG. 18).

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats or prevents obesity or obesity-related disorders.

In certain embodiments, administering the compound(s) described herein to the subject allows for administering a lower dose of the additional therapeutic agent as compared to the dose of the additional therapeutic agent alone that is required to achieve similar results in treating or preventing a obesity or obesity-related disorders in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Skin Disorders

In various embodiments, methods of treating skin disorders or improving scalp health are provided. The method includes topically administering at least one vitamin D₃ analog, as described herein, or by directly administering a TSLP isoform to a subject suffering from a skin disorder. The types of skin disorder that can be treated with these methods is not particularly limited, and any disorder that benefits from elevated sebum secretion and/or increased skin barrier function. In certain embodiment, skin disorders include eczema/atopic dermatitis, dry skin-associated dermatitis, dry skin (xerosis cutis), ichthyosis (all forms), recurrent skin infections, wrinkles (aging skin), and hair loss and/or hair growth deficiency (such as, but not limited to alopecia, such as but not limited to androgenic alopecia).

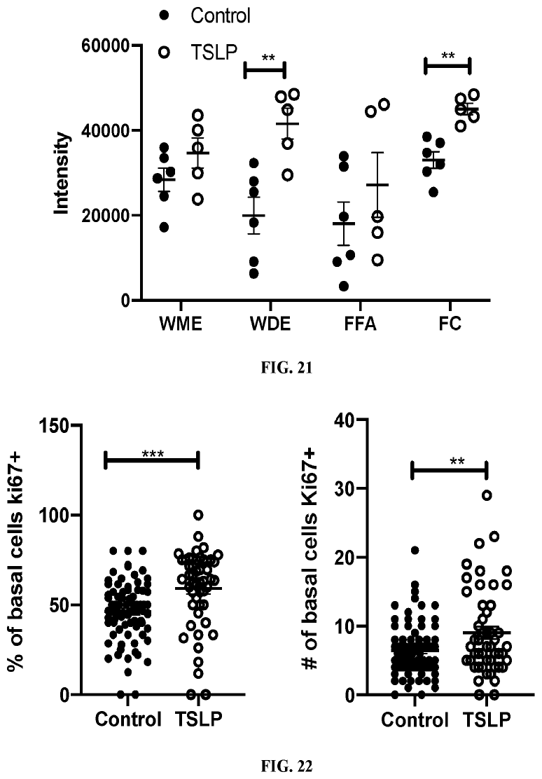

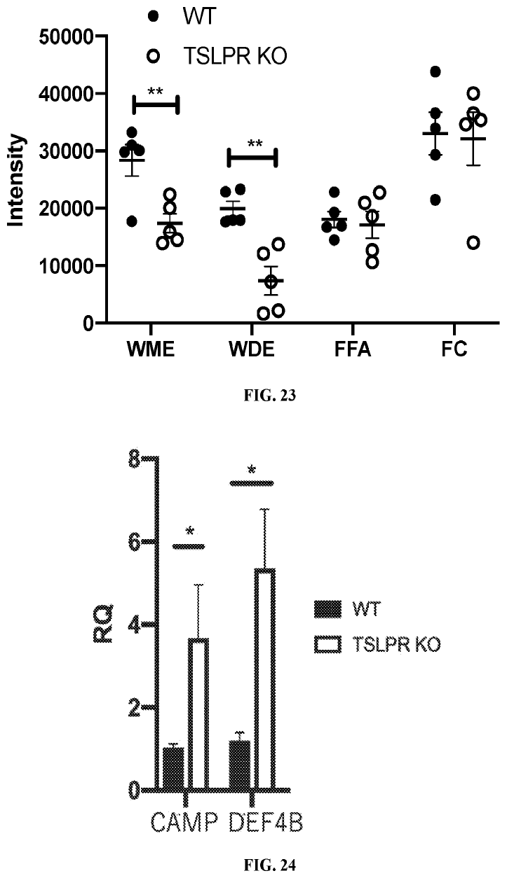

Without being bound by theory, the mechanism by which this adipose tissue loss occurred can be related to increased sebum production from TSLP-overexpressed mice. Although not apparent until about week 4, mice that are treated with AAV8-TSLP develop oily fur. To test this more directly, the lipids were extracted from the shaved fur of AAV8-TSLP compared to AAV8-control injected mice. Consistent with the oily fur, the mice exhibited increased lipids that are specifically found in sebum (wax monoesters and wax diesters) on their fur, suggesting that the mice produced more sebum when TSLP was overexpressed (FIG. 21). Moreover, immunohistochemistry of the sebaceous glands revealed an increased proportion of KI67+ basal layer sebocytes (FIG. 22), suggesting that the glands were more active. Next, to test whether TSLP possessed a physiological role in controlling sebum release, the fur of unmanipulated WT or TSLP-R KO mice was shaved and the amount of sebum lipid components quantified. TSLP-R KO mice displayed a decreased amount of sebum-specific lipid components (wax monoesters and wax diesters) compared to WT mice (FIG. 23). These data suggest that sebum release can be increased pharmacologically by TSLP-elevating agents (or by TSLP itself) and can be inhibited by blocking TSLP. TSLP also promoted barrier function by increasing the expression of anti-microbial peptides from the skin (FIG. 24)

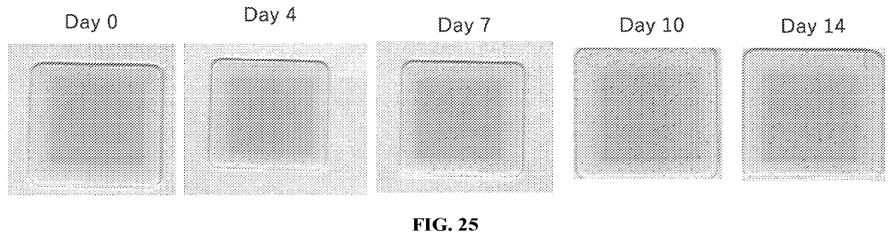

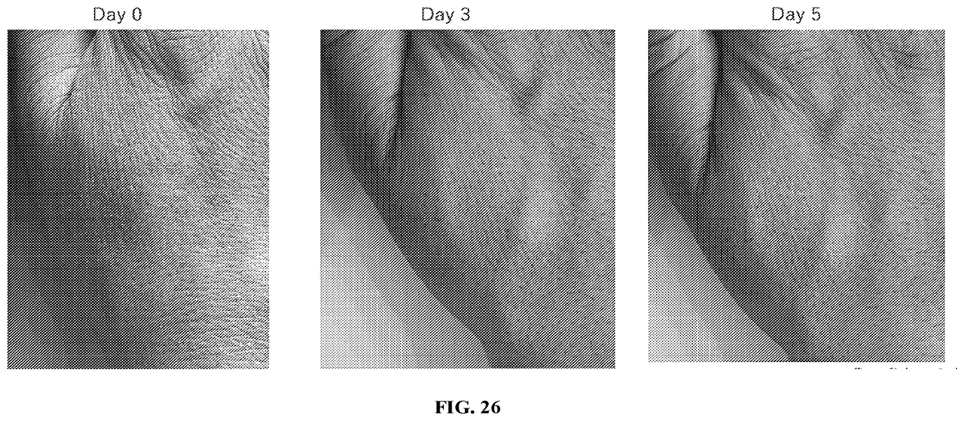

Topical MC903 has the desired effect in human. An individual showed increasing amounts of sebum on the forehead after topical MC903 application on the arm (FIG. 25). The eczematous dry skin of a hand exhibited marked improvement after topical MC903 application on the arm (FIG. 26). Given that a healthy scalp provides a better environment for hair growth and that eczema/atopic dermatitis yields defective hair growth (onlinelibrary dot wiley dot com/doi/full/10 dot 1002/jemt dot 21101), hair growth would also be promoted by topical MC903 treatment in a remote manner. Indeed, substantial recovery of hair growth was observed after 2 cycles of 2-week topical MC903 application on the arm (FIG. 27). Together, these data suggest that at least one vitamin D₃ analog, as described herein, or direct administration of a TSLP isoform remotely improves eczema/atopic dermatitis, dry skin-associated dermatitis, dry skin (xerosis cutis), wrinkles (aging skin), and ichthyosis (all forms) by promoting sebum secretion. In certain embodiments, at least one vitamin D₃ analog, as described herein, or direct administration of a TSLP isoform remotely treats, ameliorates, and/or prevents alopecia (such as, but not limited to, androgenetic alopecia). Such a treatment (vitamin D₃ analog and/or direct administration of a TSLP isoform) will treat or prevent recurrent skin infections. In certain embodiments, such a treatment promotes anti-microbial peptides contained within sebum. In certain embodiments, such a treatment promotes hair growth or prevents or minimizes hair loss by creating a healthier scalp environment.

Unexpectedly, the method described herein can treat a skin disorder when the at least one vitamin D₃ analog is administered to a healthy area of skin. That is, the vitamin D₃ analog is not administered on a skin lesion or other manifestation of the disorder on the skin, but rather to a site on the subject's skin that is healthy. In one embodiment, the vitamin D₃ analog is calcipotriol. The vitamin D₃ analog can be administered in a topical composition such as a cream, gel, lotion, or patch. In some embodiments, the vitamin D₃ analog is the only active agent in the topical composition. The vitamin D₃ analog can be present in an amount of about 0.0001 to about 10% (w/w) relative to the amount of inactive components in the topical composition. In various embodiments, the vitamin D₃ analog can be present in an amount of about 0.0001 to about 10%, about 0.001 to about 10%, about 0.01 to about 10%, about 0.1 to about 10%, about 0.0001 to about 5%, about 0.0001 to about 2%, about 0.0001 to about 1%, about 0.001 to about 1%, or about 0.01 to about 1% (w/w). The topical composition can include pharmaceutically or cosmetically acceptable excipients, such as, without limitation, perfumes, colorants, emulsifiers, skin penetration enhancers, viscosity modifiers, and the like.

Eye Disorders

In various embodiments, methods of treating eye disorders are provided. The method includes topically administering at least one vitamin D₃ analog, as described herein, or by directly administering a TSLP isoform to a subject suffering from an eye disorder. In cases of topical administration, the vitamin D₃ analog can be administered distal from the eye, so that no direct contact between the vitamin D₃ analog and the eye is made. The types of eye disorders that can be treated with these methods is not particularly limited, and any disorder that benefits from elevated meibum secretion and/or increased eye barrier function. In one embodiment, eye disorders include dry eye syndrome, kerartoconjunctivitis sicca, keratitis sicca, dysfunctional tear syndrome, age-related dry eye syndrome, medication-related dry eye syndrome, menopausal dry eye syndrome, contact lens-associated dry eye, environment-induced dry eye, dysfunctional eyelid-induced dry eye, autoimmune-associated dry eye (Sjogren's syndrome, rheumatoid arthritis, systemic lupus erythematosus), and infection-related conjunctivitis.

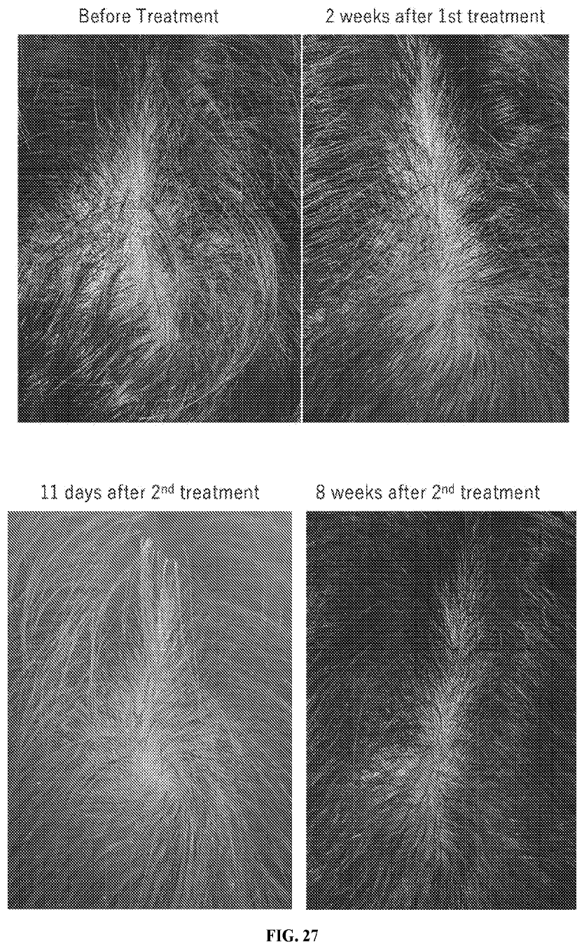

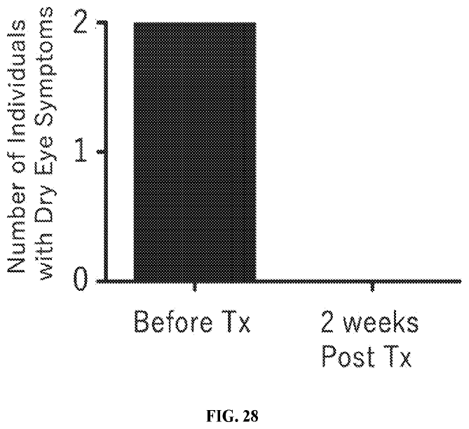

Given that mebomian gland function, which increases moisturization of the eye, is regulated in a similar manner to sebaceous glands, it was possible that dry eye symptoms would also be improved by topical MC903 treatment in a remote manner. Indeed, there was improvement in dry eye symptoms subjectively in two individuals that reported dry eye symptoms before initiation of therapy (FIG. 28).

Methods of Treating, Ameliorating, and/or Preventing Skin Disorders by Reducing TSLP Levels

In various embodiments, a method of treating, ameliorating, and/or preventing a skin disorder comprises administering a TSLP inhibiting agent to a subject. Without being bound by theory, a TSLP inhibiting agent can inhibit the secretion of sebum by the skin. Sebum is an oily substance secreted from hair follicles. Sebum is important for maintaining moisture in our skin and to provide barrier function against pathogens. However, the increased secretion of sebum can lead to undesirable diseases such as acne vulgaris, hidradenitis suppurativa, and seborrheic dermatitis. Hereby, a novel factor that controls sebum production is identified.

The first indication of a role of TSLP in controlling sebum production came from studies showing that the forced expression of TSLP in the liver of mice by an adeno-associated virus 8 expressing TSLP (AAV8-TSLP) caused selective adipose tissue loss. Although only slight weight loss was observed in normal chow-fed mice treated with AAV8-TSLP (FIG. 8A), a near complete loss of the epididymal white adipose tissue (eWAT) and inguinal white adipose tissue (iWAT) was observed between Day 7 and Day 14 post injection of AAV8-TSLP compared to AAV8-control in a dose-dependent manner (FIGS. 8B-8C). This effect was specific to white adipose tissue, since no differences in brown adipose tissue (BAT) or muscle weight were seen in AAV8-TSLP compared to AAV8-control-injected mice (FIGS. 8D-8E). The white adipose tissue loss caused by AAV8-TSLP occurred in a dose-dependent manner (FIG. 20)

Together, these data suggest that TSLP positively controls sebum release. Thus, blocking TSLP (e.g., by a neutralizing antibody) can decrease sebum release and be beneficial in the treatment of disorders that are caused by increased sebum release. Skin disorders that can be treated by TSLP inhibiting agents include acne vulgaris, seborrheic dermatitis, and hidradenitis supparativa. Non-limiting examples of a TSLP inhibiting agent include antibodies, small molecules, siRNA, shRNA, and miRNA. In humans, TSLP exists in two forms, a short form (sfTSLP) and a long form (lfTSLP). In one embodiment, the TSLP inhibiting agent can bind to and inhibit sfTSLP, lfTSLP, or both.

### Antibody Inhibitors

The disclosure also provides an inhibitor of sfTSLP or lfTSLP comprising an antibody, or antibody fragment, specific for sfTSLP or lfTSLP. That is, the antibody can inhibit sfTSLP or lfTSLP to provide a beneficial effect, such as reducing sebum secretion. Suitable antibodies include, for example, tezepelumab (also known as MEDI9929 and AMG 157; CAS number 1572943-04-4).

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain F_{V} molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

By way of a non-limited example, an antibody useful within the invention can bind to circulating sfTSLP or lfTSLP. As will be understood by one skilled in the art, any antibody that may recognize and specifically bind to circulating sfTSLP or lfTSLP is useful in the present invention. The invention should not be construed to be limited to any one type of antibody, either known or heretofore unknown, provided that the antibody can specifically bind to circulating sfTSLP or lfTSLP, and prevent or minimize biological activity of the sfTSLP or lfTSLP.

Methods of making and using such antibodies are well known in the art. For example, the generation of polyclonal antibodies may be accomplished by inoculating the desired animal with the antigen and isolating antibodies which specifically bind the antigen therefrom. Monoclonal antibodies directed against full length or peptide fragments of a protein or peptide may be prepared using any well-known monoclonal antibody preparation procedures, such as those described, for example, in Harlow et al. (1989, Antibodies, A Laboratory Manual, Cold Spring Harbor, N.Y.) and in Tuszynski et al. (1988, Blood 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein. However, the invention should not be construed as being limited solely to methods and compositions including these antibodies, but should be construed to include other antibodies, as that term is defined elsewhere herein.

In some instances, it is desirable to prepare monoclonal antibodies from various mammalian hosts, such as rodents (e.g., mice), primates (e.g., humans), and so forth. Descriptions of techniques for preparing such monoclonal antibodies are well known and are described, for example, in Harlow et al., ANTIBODIES: A LABORATORY MANUAL, COLD SPRING HARBOR LABORATORY, Cold Spring Harbor, N.Y. (1988); Harlow et al., USING ANTIBODIES: A LABORATORY MANUAL, (Cold Spring Harbor Press, New York, 1998); Breitling et al., RECOMBINANT ANTIBODIES (Wiley-Spektrum, 1999); and Kohler et al., 1997 Nature 256: 495-497; and U.S. Pat. Nos. 5,693,762; 5,693,761; 5,585,089; and 6,180,370.

Nucleic acid encoding an antibody obtained using the procedures described herein may be cloned and sequenced using technology that is available in the art, and is described, for example, in Wright et al. (Critical Rev. Immunol. 1992, 12:125-168) and the references cited therein. Further, the antibody useful within the invention may be “humanized” using the technology described in Wright et al. (supra) and in the references cited therein, and in Gu et al. (Thrombosis and Hematocyst 1997, 77:755-759).

Alternatively, antibodies may be generated using phage display technology. To generate a phage antibody library, a cDNA library is first obtained from mRNA that is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell that expresses the corresponding antigen, the bacteriophage will bind to the cell. Bacteriophage that do not express the antibody will not bind to the cell. Such panning techniques are well known in the art and are described for example, in Wright et al. (Critical Rev. Immunol. 1992, 12:125-168).

Processes such as those described herein have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage that display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage that encode the Fab portion of an antibody molecule. However, the invention should not be construed to be limited solely to the generation of phage encoding Fab antibodies. Rather, phage that encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J Mol Biol 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J Mol Biol 248:97-105).

The invention encompasses polyclonal, monoclonal, synthetic antibodies, and the like. One skilled in the art would understand, based upon the disclosure provided herein, that an important feature of the antibody useful within the invention is that the antibody specifically bind with a circulating protein.

### Small Molecule Inhibitors

In certain embodiments, the TSLP inhibiting agent comprises a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

### Nucleic Acid Inhibitors

In certain embodiments, the TSLP inhibiting agent comprises an isolated nucleic acid. In other embodiments, the inhibitor is an siRNA or antisense molecule, which inhibits sfTSLP or lfTSLP. In yet other embodiments, the nucleic acid comprises a promoter/regulatory sequence such that the nucleic acid is preferably capable of directing expression of the nucleic acid. Thus, the invention provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, sfTSLP or lfTSLP can be inhibited by way of inactivating and/or sequestering sfTSLP or lfTSLP. As such, inhibiting the activity of sfTSLP or lfTSLP can be accomplished by using a transdominant negative mutant.

In certain embodiments, siRNA is used to decrease the level of sfTSLP or lfTSLP protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; Engelke, Ed., RNA Interference (RNAi) Nuts & Bolts of RNAi Technology, DNA Press, Eagleville, P A (2003); and Hannon, Ed., RNAi A Guide to Gene Silencing, Cold Spring Harbor Laboratory Press, Cold Spring Harbor, N.Y. (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of sfTSLP or lfTSLP using RNAi technology.

In certain embodiments, the invention provides a vector comprising an siRNA or antisense polynucleotide. In other embodiments, the siRNA or antisense polynucleotide inhibits the expression of sfTSLP, lfTSLP, or both. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In certain embodiments, the vector of the invention is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid that is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide has certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In certain embodiments, an antisense nucleic acid sequence expressed by a plasmid vector is used to inhibit sfTSLP or lfTSLP protein expression. The antisense expressing vector is used to transfect a mammalian cell or the mammal itself, thereby causing reduced endogenous expression of sfTSLP or lfTSLP.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In certain embodiments, a ribozyme is used to inhibit sfTSLP or lfTSLP protein expression. Ribozymes useful for inhibiting the expression of a target molecule may be designed by incorporating target sequences into the basic ribozyme structure which are complementary, for example, to the mRNA sequence encoding sfTSLP or lfTSLP. Ribozymes targeting sfTSLP or lfTSLP, may be synthesized using commercially available reagents (Applied Biosystems, Inc., Foster City, Calif.) or they may be genetically expressed from DNA encoding them.

In various embodiments, a method of treating a skin disorder comprises administering a TSLP stimulating agent to a subject.

### Combination Therapies

The compounds useful within the methods described herein can be used in combination with one or more additional therapeutic agents useful for treating obesity or obesity-related disorders. These additional therapeutic agents may comprise compounds that are commercially available or synthetically accessible to those skilled in the art. These additional therapeutic agents are known to treat, prevent, or reduce the symptoms, of obesity or obesity-related disorders.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of obesity or obesity-related disorders. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat obesity or obesity-related disorders in the patient. An effective amount of the therapeutic compound necessary to achieve a therapeutic effect may vary according to factors such as the state of the disease or disorder in the patient; the age, sex, and weight of the patient; and the ability of the therapeutic compound to treat obesity or obesity-related disorders in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In certain embodiments, the compositions described herein are applied to the skin of the subject, using for example a patch, an adhesive membrane, a self-sticking membrane, a lotion, a paste, and the like. In some embodiments, the compositions described herein are administered by injection, such as by subcutaneous, intramuscular, or intravenous injection.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of obesity, or an obesity-related disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

In various embodiments, a therapeutic amount of a vitamin D₃ analog is from about 0.01 nmol to about 20 nmol per cm² of treated skin. The dose of the vitamin D₃ analog can be from about 0.05 nmol/cm² to about 10 nmol/cm², 0.1 nmol/cm² to about 5 nmol/cm², or 0.1 nmol/cm² to about 2.5 nmol/cm², wherein the area corresponds to the surface area of treated skin. The dose of the vitamin D₃ analog can be about 0.01 nmol/cm², 0.05 nmol/cm², 0.1 nmol/cm², 0.2 nmol/cm², 0.4 nmol/cm², 0.6 nmol/cm², 0.8 nmol/cm², 1 nmol/cm², 1.5 nmol/cm², 2 nmol/cm², 3 nmol/cm², 4 nmol/cm², 5 nmol/cm², 6 nmol/cm², 7 nmol/cm², 8 nmol/cm², 9 nmol/cm², 10 nmol/cm², 12 nmol/cm², 14 nmol/cm², 16 nmol/cm², 18 nmol/cm², or 20 nmol/cm², wherein the area corresponds to the surface area of treated skin.

The vitamin D₃ analog can be applied to any particular skin surface area, depending on the needs of the subject. In various embodiments, the vitamin D₃ analog can be applied to a skin area ranging from 5 cm² to about 2500 cm², about 10 cm² to about 2000 cm², about 20 cm² to about 1500 cm², about 30 cm² to about 1000 cm², 40 cm² to about 750 cm², 50 cm² to about 700 cm², 60 cm² to about 650 cm², about 70 cm² to about 600 cm², 80 cm² to about 550 cm², 90 cm² to about 500 cm², or 100 cm² to about 450 cm². The vitamin D₃ analog, in various embodiments, can be applied to a skin area of about 5 cm², 10 cm², 15 cm², 20 cm², 25 cm², 30 cm², 35 cm², 40 cm², 45 cm², 50 cm², 55 cm², 60 cm², 65 cm², 70 cm², 75 cm², 80 cm², 85 cm², 90 cm², 95 cm², 100 cm², 110 cm², 120 cm², 130 cm², 140 cm², 150 cm², 160 cm², 170 cm², 180 cm², 190 cm², 200 cm², 225 cm², 250 cm², 275 cm², 300 cm², 350 cm², 400 cm², 450 cm², 500 cm², 550 cm², 600 cm², 650 cm², 700 cm², 750 cm², 800 cm², 850 cm², 900 cm², 950 cm², 1000 cm², 1250 cm², 1500 cm², 1750 cm², 2000 cm², 2250 cm², or 2500 cm².

The amount of the vitamin D₃ analog applied to a given skin area can be about 0.005% to about 10% w/w, about 0.01% to about 5% w/w, about 0.05% to about 5% w/w, about 0.1% to about 5% w/w, or about 0.5% to about 5% w/w, with respect to any pharmaceutical composition of the D₃ analog described herein. Thus, for example, a topical pharmaceutical composition of the vitamin D₃ analog can include 0.005% to about 10% w/w of the vitamin D₃ analog, with the remainder being pharmaceutically acceptable carrier and/or excipients. In various embodiments, the amount of the vitamin D₃ analog applied to a given skin area can be about 0.005%, 0.01%, 0.05%, 0.1%, 0.%, 0.3%, 0.4%, 0.5%, 0.6%, 0.7%, 0.8%, 0.9%, 1%, 1.2%, 1.4%, 1.6%, 1.8%, 2%, 3%, 4%, 5%, 6%, 7%, 8%, 9%, or 10% w/w with respect to any pharmaceutical composition of the D₃ analog described herein.

The amount of a TSLP polypeptide isoform or a viral vector that includes a TSLP-expression sequence administered to a subject and that is effective to achieve the weight loss described herein is from about 0.1 mg/kg to about 500 mg/kg, about 0.5 mg/kg to about 400 mg/kg, about 1 mg/kg to about 300 mg/kg, about 5 mg/kg to about 200 mg/kg, or about 10 mg/kg to about 100 mg/kg. The effective amount of a TSLP polypeptide isoform or a viral vector that includes a TSLP-expression sequence to achieve the weight loss described herein is, in various embodiments, at least, greater than, or less than about 0.1 mg/kg, 0.5 mg/kg, 1 mg/kg, 2 mg/kg, 3 mg/kg, 4 mg/kg, 5 mg/kg, 0.1 mg/kg, 6 mg/kg, 7 mg/kg, 8 mg/kg, 9 mg/kg, 10 mg/kg, 15 mg/kg, 20 mg/kg, 40 mg/kg, 60 mg/kg, 80 mg/kg, 100 mg/kg, 150 mg/kg, 200 mg/kg, 250 mg/kg, 300 mg/kg, 350 mg/kg, 400 mg/kg, 450 mg/kg, or about 500 mg/kg. In various embodiments, an amount of a TSLP polypeptide isoform or a viral vector that includes a TSLP-expression sequence to achieve the weight loss described herein is an amount that results in circulating TSLP levels in the subject of about 5 to about 40 ng/mL.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In various embodiments, the vitamin D₃ analog is administered using a dosing schedule that has a treatment week followed by a no-treatment week. During the no-treatment week no vitamin D₃ analog is administered. During the treatment week, the vitamin D₃ analog can be administered daily (7 days of treatment), or every other day (3 or 4 days of treatment).

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples, which are offered by way of illustration. The scope of the present application is not limited to the Examples given herein.

### Example 1: MC903 Prevents Weight Gain and Improve Metabolic Parameters of HFD-Fed Mice

Wildtype C57BL/6 mice were fed a normal chow (NC) or 40% high fat diet (HFD) for 12 weeks. The mice were topically treated with either vehicle (EtOH) or MC903 (2 nmol/ear) on both ears Monday, Wednesday, and Friday on odd weeks. (A) Mice were weighed weekly and % change from baseline is plotted as mean±SEM. (B) The mice were euthanized on Week 12 and the epididymal fat pads were weighed. (C, D) Glucose tolerance test (GTT) was performed on Week 12 by injection of glucose and subsequent measurements of blood glucose levels. Homeostatic model assessment-insulin resistance (HOMA-IR) analysis was performed after an overnight fast at Week 12. *, **, and *** indicate statistical significance of p<0.05, p<0.01, and p<0.001, respectively by Student t-test or ANOVA.

### Example 2. MC903 Prevents HFD-Induced Hepatosteatosis

C57BL/6 mice were fed a normal chow (NC) or 40% high fat diet (HFD) for 12 weeks. The mice were topically treated with either vehicle (EtOH) or MC903 (2 nmol/ear) on both ears Monday, Wednesday, and Friday on odd weeks. Shown are representative histological images of the liver on Week 12.

### Example 3. MC903 does not Prevent Weight Gain or Improve Metabolic Parameters in HFD-Fed TSLP-R KO Mice

TSLP-R knock out (KO) mice were fed a normal chow (NC) or 40% high fat diet (HFD) for 12 weeks. The mice were topically treated with either vehicle (EtOH) or MC903 (2 nmol/ear) on both ears Monday, Wednesday, and Friday on odd weeks. Mice were weighed on the indicated time points and plotted as mean±SEM. Glucose tolerance test (GTT) was performed on Week 12 by injection of glucose and subsequent measurements of blood glucose levels. AUC analysis is performed on the GTT curves. * indicates statistical significance of p<0.05 by Student t-test.

### Example 4. AAV8-TSLP Induces Weight Loss in HFD-Fed Mice

C57BL/6 mice were fed a 40% high fat diet (HFD) for 4 weeks. The mice were injected intravenously with either AAV8-control (Control AAV) or AAV8-TSLP (mTSLP-AAV) on Day 0. The mice were weighed weekly and % change from baseline is plotted as mean±SEM. ** and *** indicate statistical significance of p<0.01 and p<0.001, respectively.

### Example 5. AAV8-TSLP Induces Weight Loss in Previously Obese HFD-Fed Mice

C57BL/6 mice were fed a 40% high fat diet (HFD) for 10 weeks. The mice were then injected intravenously with either AAV8-control or AAV8-TSLP at Week 10. The mice were kept on a HFD and weighed weekly and % change from baseline is plotted as mean±SEM. *, **, and *** indicate statistical significance of p<0.05, p<0.01, and p<0.001, respectively by Student t-test or ANOVA.

### Example 6. AAV8-TSLP Induces Loss of Subcutaneous Fat

C57BL/6 mice were fed a 40% high fat diet (HFD) for 10 weeks. The mice were then injected intravenously with either AAV8-control or AAV8-TSLP at Week 10. The mice were kept on a HFD for 6 weeks and euthanized for histological analysis. Shown are representative histological images of the skin of mice.

### Example 7. AAV8-TSLP Induces Weight Loss in Ob/Ob Mice

Ob/Ob mice were injected with either AAV8-control or AAV8-TSLP and fed a normal chow for 6 weeks. The mice weighed weekly and % change from baseline is plotted as mean±SEM. * and *** indicate statistical significance of p<0.05 and p<0.001, respectively by Student t-test.

### Example 8. AAV8-TSLP Induces Selective Adipose Tissue Loss in Normal Chow-Fed Mice

C57BL/6 mice were injected intravenously with either AAV8-control or AAV8-TSLP and fed a normal chow for 14 days. (A) Mice were weighed on the indicated days and % change from baseline is plotted as mean±SEM. The mice were euthanized on the indicated days and the (B) epididymal white adipose tissue (eWAT), (C) inguinal white adipose tissue (iWAT), (D) brown adipose tissue (BAT), and (E) quadriceps weights were measured. ** and *** indicate statistical significance of p<0.01 and p<0.001, respectively by Student t-test.

### Example 9. AAV8-TSLP does not Alter Food Consumption of Gut Absorption

C57BL/6 mice were injected intravenously with either AAV8-control or AAV8-TSLP and fed a normal chow or HFD for 12 weeks. (A) Average weekly food consumption is plotted as mean±SEM. On day 10 post AAV8 injection, mice were oral gavaged with a fixed amount of (B) glucose or (C) olive oil. Subsequent glucose levels and triglyceride levels were measured and plotted over time.

### Example 10. AAV8-TSLP-Treated Mice Excrete Less Fecal Energy Compared to AAV8-Control-Treated Mice

C57BL/6 mice were injected intravenously with either AAV8-control or AAV8-TSLP and fed a normal chow for 9 days. Stool was collected and food consumption was measured in individually caged mice between days 9 and 11. The collected stool was subjected to bomb calorimetry for measurement of fecal calories. (A) The fecal energy content per day of AAV8-control or AAV8-TSLP treated mice is shown. (B) The net energy intake per day, as calculated by the food caloric intake subtracted by the fecal calories of AAV8-control or AAV8-TSLP treated mice is shown. Statistical analysis performed by Student t-test.

### Example 11. AAV8-TSLP Increases Food Consumption Between Days 7-10 Post Injection

C57BL/6 mice were injected intravenously with either AAV8-control or AAV8-TSLP and placed in a metabolic chamber on Days 7-10. Hourly and cumulative food consumption is plotted over the last 48 hours.

### Example 12. AAV8-TSLP does not Increase Oxygen Consumption Between Days 7-10 Post Injection

C57BL/6 mice were injected intravenously with either AAV8-control or AAV8-TSLP and placed in a metabolic chamber on Days 7-10. Hourly (left plot) and daily/half daily (right plot) oxygen consumption is plotted over the last 48 hours and 72 hours, respectively.

### Example 13. AAV8-TSLP does not Increase Carbon Dioxide Output Between Days 7-10 Post Injection

WT C57BL/6 mice were injected intravenously with either AAV8-control or AAV8-TSLP and placed in a metabolic chamber on Days 7-10. Hourly (left plot) and daily/half daily (right plot) carbon dioxide output is plotted over the last 48 hours and 72 hours, respectively.

### Example 14. AAV8-TSLP does not Increase Locomotor Activity Between Days 7-10 Post Injection

WT C57BL/6 mice were injected intravenously with either AAV8-control or AAV8-TSLP and placed in a metabolic chamber on Days 7-10. Hourly (left plot) and daily/half daily (right plot) locomotor activity is plotted over the last 48 hours and 72 hours, respectively.

### Example 15. AAV8-TSLP Causes Oily Fur in Previously Obese HFD-Fed Mice

C57BL/6 mice were fed a 40% high fat diet (HFD) for 10 weeks. The mice were then injected intravenously with AAV8-TSLP at Week 10. The mice were kept on a HFD for another 6 weeks. A representative photograph of the mice on Week 6 post AAV8 injection is shown.

### Example 16. AAV8-TSLP Induces Weight Loss in HFD-Fed Mice with a Hematopoietic System that is TSLP Responsive

C57BL6 or TSLP-R KO mice were irradiated with 1000 cGy and injected intravenously with bone marrow cells from C57BL/6 or TSLP-R knockout (KO) mice. 4 weeks after injection of bone marrow, the mice were injected intravenously with either AAV8-control or AAV8-TSLP and fed a 40% high fat diet (HFD) for 3 weeks. The mice were weighed weekly and % change from baseline is plotted as mean±SEM.

### Example 17. AAV8-TSLP Induces Weight Loss in a T Cell-Dependent Manner

(A) Recombinase activating gene-1 (RAG1) KO mice and (B) T cell receptor (TCR)-beta KO mice were injected with either AAV8-control or AAV8-TSLP. RAG1 KO mice lack T and B cells. TCR-beta KO mice lack T cells only. The epididymal white adipose tissue (eWAT) was weighed on Day 14 post injection. Data are plotted as mean±SEM.

### Example 18. Non-Limiting Model of TSLP-Induced Adipose Tissue Loss

The elevation of systemic TSLP can be accomplished by topical treatment with MC903, injection of recombinant TSLP, or gene therapy using a viral vector. Increased systemic TSLP levels leads to skin lipid secretion, which increases the fat energy demands of the organism. This causes liberation of fat stores (lipolysis) in adipose and other fat-harboring tissues. This eventually leads to weight loss and the reversal of obesity. Thus, this strategy is beneficial in the treatment of obesity and obesity-related disorders such as non-alcoholic steatohepatitis (NASH) and type 2 diabetes (T2D).

### Example 19: Thymic Stromal Lymphopoietin Protects 1 Against Obesity Through Sebum Hypersecretion

The present results show that Thymic Stromal Lymphopoietin (TSLP), a type II cytokine that activates ILC2s and eosinophils, induces selective white adipose loss and protects against obesity and associated complications, including type II diabetes and non-alcoholic steatohepatitis (NASH). Surprisingly, however, the induction of fat loss was dependent on T cells and not on ILC2s or eosinophils. Furthermore, the adipose loss was not secondary to increased thermogenesis and energy expenditure, but rather involved the hypersecretion of sebum. Inhibition of sebum secretion or depletion of T cells prevented TSLP-driven fat loss. In addition to the pharmacological effects of TSLP, the present data revealed a homeostatic role for TSLP and T cells in the regulation of sebum secretion. Together, this study demonstrates that adipose loss can be achieved by sebum hypersecretion and uncovers a previously unknown role of TSLP and T cells in controlling sebum release.

### Methods

Mice. C57BL/6 and B6-LY5.1/Cr mice were purchased from Charles River Laboratories (stock no. 556 and 564). Ob/ob, SCD1 KO, RAG/IL2Rⱼ DKO, ROSA-DTA (ROSA-Stop-flox-DTA), and CD11cCre mice were purchased from Jackson Laboratories (stock no. 000632, 005956, 014593, 009669, and 008068). EoCre mice were created as previously described (Doyle, et al., 2013, Leukoc Biol 94:17-24). CD11cCre and ROSA-DTA mice were crossed to generate DC-deficient mice, and EoCre and ROSA-DTA mice were crossed to generate eosinophil-deficient mice. TSLPR KO mice, as previously described (Al-Shami, et al., 2004, J Exp Med 200:159-168) were a gift from Warren Leonard (NIH) and bred and maintained in othe facility. E-Beta KO, RAG2 KO (RAG KO), FOXP3-GFP, and FOXP3-DTR mice were also bred and maintained in the facility. For CD4⁺ and CD8⁺ T cell depletion, antibodies against CD4 (BioXCell, BE0003-1 clone GK1.5) and CD8 (BioXCell, BE006, clone 2.43) were administered via intraperitoneal injection at a dose of 200 μg/mouse on days 0, 2, 4, 8, and 12 or on days 14, 16, 18, 22, and 26 following AAV injection. FOXP3-GFP and FOXP3-DTR mice were administered diphtheria toxin (Santa Cruz Biotechnology, sc-391135) intraperitoneally at a dose of 10 μg/kg weight on days −2, 0, 2, 4, 6, 8, 10, and 12 after AAV injection. For all NC-fed mouse experiments, mice were harvested 2 weeks after AAV injection and eWAT (bilateral), iWAT (bilateral), BAT (bilateral), and quadriceps muscle (left quadriceps) masses were measured. Ob/ob mice were given AAV injection for 5 weeks starting from 12 weeks of age. Unless otherwise specified, all mice were males aged 8 to 10 weeks old at the time of use, and were housed in pathogen-free conditions.

AAV injections. Control-AAV (AAV8.TBG.PI.Null.bGH) and TSLP-AAV (AAV8.TBG.PI.mTSLP.IRES.eGFP.WPRE.bGH) were generated by the Penn Vector Core. Mice were injected intravenously with 3×10¹⁰ genome copies of AAV, equivalent to serum TSLP levels of 40 ng/mL.

BM chimeras. Femurs and hip bones were isolated from donor mice and crushed with a mortar and pestle to obtain bone marrow. Red cells were lysed with ACK lysing buffer (ThermoFisher, A1049201) and bone marrow was filtered through a 70 μm filter (Sigma, CLS431751). Host mice were irradiated with 1,000 cGy and injected intravenously with 2×10⁶ donor bone marrow cells. Four weeks later, BM chimeras were injected with AAV.

Adoptive transfers. WT or TSLPR KO splenic T cells were isolated using a T cell negative selection kit (STEMCELL Technologies, 19851) and then sorted for CD19⁻/B220⁻/NK1.1⁻/CD11c⁻/CD11b⁻ and CD90.2⁺/CD4⁺ or CD8⁺ cells using a FACSAria cell sorter (BD Biosciences). 2×10⁶ sorted cells were transferred intravenously into RAG KO or TSLPR KO mice. Four weeks later, adoptively transferred mice were injected with AAV.

HFD and MCDD models. Mice were either fed a HFD consisting of 45 kcal % fat (Research Diets, D12451) or an MCDD consisting of 60 kcal % fat with 0.1% methionine and no added choline (Research Diets, A06071302). For HFD models, mice were either injected with AAV at day 0 or after 10 weeks of being on HFD while continuing on HFD for an additional 4 weeks (HFD for 14 weeks with AAV injected at week 10). Mice were weighed weekly. For MCDD models, mice were injected with AAV after 4 weeks of diet while continuing on MCDD for an additional 4 weeks (MCDD for 8 weeks, AAV injection at week 4).

In vivo metabolic analysis. Mice were individually housed and monitored during days 9-11 post AAV injection by the University of Pennsylvania's Rodent Metabolic Phenotyping Core, using the OxyMax Comprehensive Laboratory Animal Monitoring System (CLAMS). Mice were fed NC and maintained on a standard 12:12 light-dark cycle at 24° C. with ad libitum access to food and water. Accumulated feces were collected from singly housed mice on days 9-11 after AAV injection and bomb calorimetry of the feces was performed by the University of Michigan Mouse Phenotyping Core. Fat mass and lean mass were measured by ¹H-NMR spectroscopy at days 0 and 14 following AAV injection. For GTTs, mice were fasted overnight for 14-16 hours, and injected intraperitoneally with 10 μl/g body weight of a 20% w/v dextrose solution. Blood glucose level was measured from blood collected from the tail vein at 0, 15, 30, 60, 90, and 120 min post dextrose challenge. 0 min time-point measurements were used to determine fasting glucose and insulin levels. Blood glucose measurements were performed using a handheld glucometer (Contour, 7151G) and plasma insulin levels were determined by ELISA (Crystal Chem, 90080). HOMA-IR index values were calculated as described previously (Matthews, et al., 1985, Diabetologia 28:412-419). OGTT was performed in a similar manner as GTT, except that the dextrose solution was administered via oral gavage. OFTT was performed as previously described (Wada, et al., 2013, Gastroenterology 144:369-380) and blood triglyceride levels were measured via the Infinity Triglyceride kit (ThermoFisher, TR22421). Serum ALT assays were performed by the Translational Core Laboratory of the Children's Hospital of Philadelphia (CHOP) Research Institute. For urinalysis, urine was collected from mice 10 days after AAV injection and 100 μl of urine was placed on urine dipsticks (Ketostix, 2881; Chemstrip 2GP, 11895397) for colorimetric analysis.

Body condition scoring. For BCS scoring (Cooke, et al., 1996, Blood 88:3230-3239), mice were scored on days 0, 3, 7, 10 and 14 after AAV injection with grading on a scale of 0-2 in 5 categories as follows: 1) Weight Loss: 2 points if >25%, 1 point if 10-25%, 0 points if <10%, 2) Hunching: 2 points if severe hunching that impairs movement, 1 point if hunched at rest, 0 points if normal posture, 3) Visible Lethargy: 2 points if stationary unless stimulated, 1 point if mild to moderately decreased movement, 0 points if normal, 4) Lethargy to touch: 2 points if stationary when stimulated, 1 point if mild to moderately to decreased movement upon stimulation, 1 point if normal, 5) Ruffled fur: 2 points if severe ruffling and poor grooming, 1 point if mild to moderate ruffling, 0 points if normal. Points from each category were summed to form the total score.

Liver TGs. 20-100 mg of liver tissue was homogenized with 10 μl/mg liver of 5% NP-40 (Igepal CA630, Sigma-Aldrich, 18896). Resulting homogenate was boiled at 80° C. for 10 min, cooled to room temperature, boiled again at 80° C. for 10 min and centrifuged. The supernatant was then diluted 1:20 with dH₂O. Triglyceride levels were quantified using the Infinity Triglyceride Reagent.

Fur lipid isolation and TLC. A 2.5 cm×5 cm area of fur was shaved from back skin and immersed in 4 mL of chloroform/methanol (2:1 v/v) followed by 4 mL of acetone. Lipid extracts were pooled, syringe filtered, dried down overnight under a stream of N₂ gas, and resuspended in equal volumes of chloroform (Sigma, 288306)/methanol (Sigma, 322415) (4:1, v/v) for loading onto TLC plates (Sigma, 100390). The TLC plates were developed three times using the following: 1) Hexane (Sigma, 296090):Isopropyl Diether (Sigma, 673803):Acetic Acid (80:20:1) up to 50% of the plate height, 2) Hexane:Benzene (Sigma, 401765) (1:1) up to 80% of the plate height, 3) Hexane up to 90% of the plate height. Plates were allowed to dry between each developing solution. Plates were sprayed uniformly with 10% cupric sulfate (Sigma, 451657)/8% phosphoric acid (Sigma, P6560) solution and then baked at 120° C. for 20 minutes to visualize lipid species. ImageJ (NIH) was used to quantify the intensity and area of the bands. The TLC non-polar lipid mixture A (Matreya, 1129) was used as a standard to identify lipids classes.

Flow cytometry analysis. Dermal sheets of ear skin were separated, minced, and incubated in Hank's balanced salt solution (ThermoFisher, 24020117) containing 0.25 mg/ml Liberase TL (Roche, 5401020001), 0.1 mg/ml DNase I (Roche, 10104159001), and 0.7 mg/ml Collagenase D (Roche, 11088882001) for 2 hours with shaking at 37° C. Contents were then strained though a 70 μm filter into a new tube containing 10 ml of PBS, centrifuged, and resuspended in PBS. Cells were stained with live/dead stain and cell surface stains at 4° C. for 15 min in PBS. Flow cytometry was performed with a LSR II or LSR Fortessa (BD Biosciences). Data were analyzed using FlowJo software (TreeStar). Staining antibodies used included: CD4 (BioLegend, 100406 and 100510), CD8α (BioLegend, 100753), CD90.2 (BioLegend, 140322), TCRb (BioLegend, 109241), CD19 (BioLegend, 115520), B220 (BioLegend, 103222), NK1.1 (BioLegend, 108714), CD3 (BioLegend, 100218), CD45.1 (BioLegend, 110739), CD45.2 (eBioscience, 56-0454-82), CD11b (BioLegend, 101215), and CD11c (BioLegend, 117318). The Live/Dead Near-IR stain (ThermoFisher, L10119) was used to exclude dead cells.

Histology. Tissues were fixed in 10% formalin at 4° C. overnight and embedded in paraffin before H&E or IHC staining. Liver sections were processed and stained by the University of Pennsylvania Cardiovascular Institute's Histology and Gene Expression Core. PicroSiriusRed stains were performed as previously described (Jeong, et al., 2018, J Clin Invest 128:1010-1025). Skin samples were processed and stained (H&E, Ki67, ORO, IHC for CD3, CD4, and CD8) by the University of Pennsylvania's Skin Histology and Characterization Core. For ORO staining of skin samples, frozen skin samples were embedded in cryosectioning medium prior to sectioning and staining. For quantification of sebocyte size, ORO lipid content, and PicroSiriusRed fibrosis, 8-10 sections at 20× magnification were captured per animal. For H&E skin sections, ImageJ was used to draw circumscribing ellipses around sebocytes to quantify area and intensity of staining. For ORO skin and PicroSiriusRed liver sections, ImageJ was used to split RGB channels, threshold positive staining, and measure % area and intensity. Integrated density was calculated as area×intensity. For quantification of Ki67 sebocyte basal cell staining, 8-10 sections per animal were imaged at 40× magnification and Ki67⁺ vs. Ki67⁻ basal cells in each sebaceous gland were counted.

Human gene expression analysis. Publicly available human skin gene expression data was obtained from www dot ncbi dot nlm dot nih dot gov/geo/query/acc dot cgi?acc=GSE98774. Gene expression values for sebum-production associated genes (SCD, FADS2, PPARg, FA2H, DGAT1, DGAT2, FABP4, FABP5, ACACA, FASN, AWAT1, ELOVL1, ELOVL3, ELOVL4, ELOVL5, MOGAT1, MOGAT2, MOGAT3) were plotted against corresponding TSLP expression values for each sample. For normalized SG gene expression, each expression of each sebum-associated gene dataset was normalized to a mean of 0 and standard deviation of 1 and then all SG genes from each sample were averaged together and plotted against the corresponding the TSLP expression. Pearson's r and Pearson linear regression analysis was conducted using Prism (GraphPad Software, Inc.).

Statistical analysis. Data are reported as mean±s.e.m. All measurements were made from distinct biological samples. For normally distributed data, statistical significance was determined by Student's t-test (two-sided) or two-way ANOVA with Sidak's post hoc test with corrections for multiple comparisons. Correlation analyses were conducted using Pearson linear regression. Statistical analyses were performed with Prism 8.

### Results

Selected results are illustrated herein.

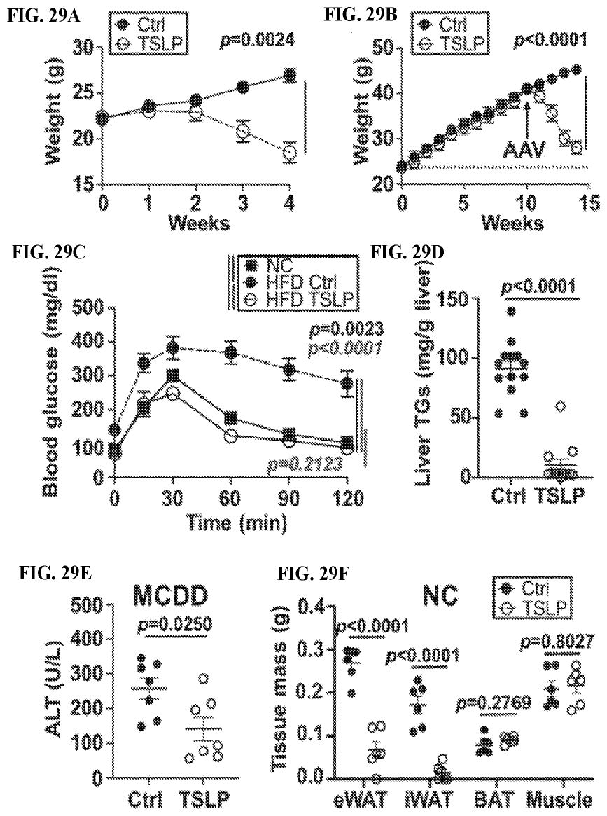

TSLP Protects Against Obesity and Obesity-Related Complications The potential effect of TSLP on obesity was tested by injecting a TSLP-expressing Adeno-Associated Virus Serotype 8 (TSLP-AAV, expression targeted to liver) to induce systemic expression of TSLP in mice fed a high fat diet (HFD). Strikingly, HFD-fed mice injected with TSLP-AAV lost weight over 4 weeks compared to mice injected with Control-AAV, which gained weight (FIGS. 29A, 33A). TSLP-AAV injection also caused weight loss in normal chow (NC)-fed hyperphagic ob/ob mice (FIGS. 33B-33C). TSLP not only prevented but also reversed obesity. TSLP-AAV-injected obese mice (mice first fed HFD for 10 weeks and then injected with TSLP-AAV while continuing on HFD) displayed weight loss, decreased visceral fat mass, and markedly improved metabolic parameters, including fasting plasma glucose and insulin levels, Homeostatic Model Assessment of Insulin Resistance (HOMA-IR), glucose tolerance, hepatic steatosis, and hepatic triglyceride (TG) levels (FIGS. 29B-29D, 33D-33J).

Given the reduction in hepatic steatosis, the effect of TSLP was also tested in a mouse model of non-alcoholic fatty liver disease (NAFLD) and NASH. To this end, mice were fed a methionine-choline-deficient diet (MCDD) for 4 weeks, and then injected with either Control-AAV or TSLP-AAV for an additional 4 weeks. TSLP-AAV-injected MCDD-fed mice had lower liver TG levels, lower serum alanine aminotransferase levels (ALT), and decreased hepatic fibrosis compared to controls (FIGS. 29E, 34A-33D).

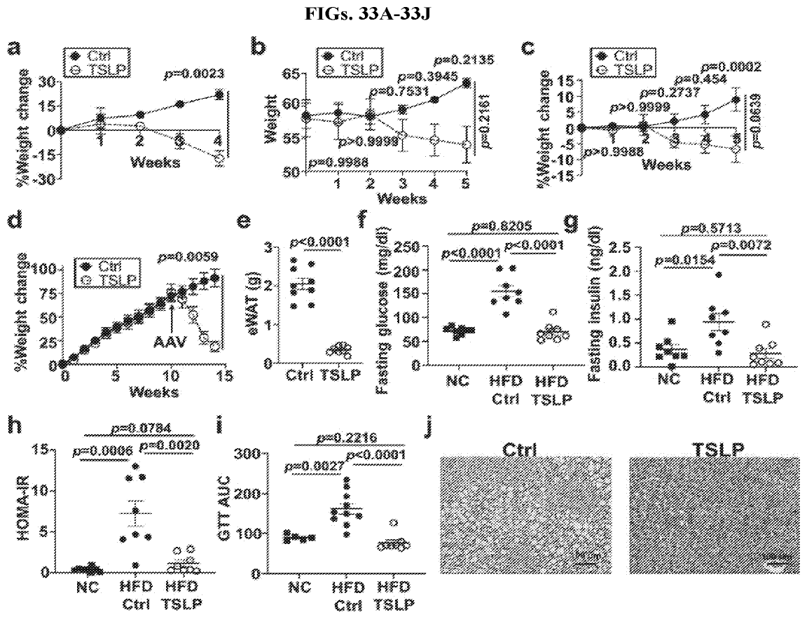

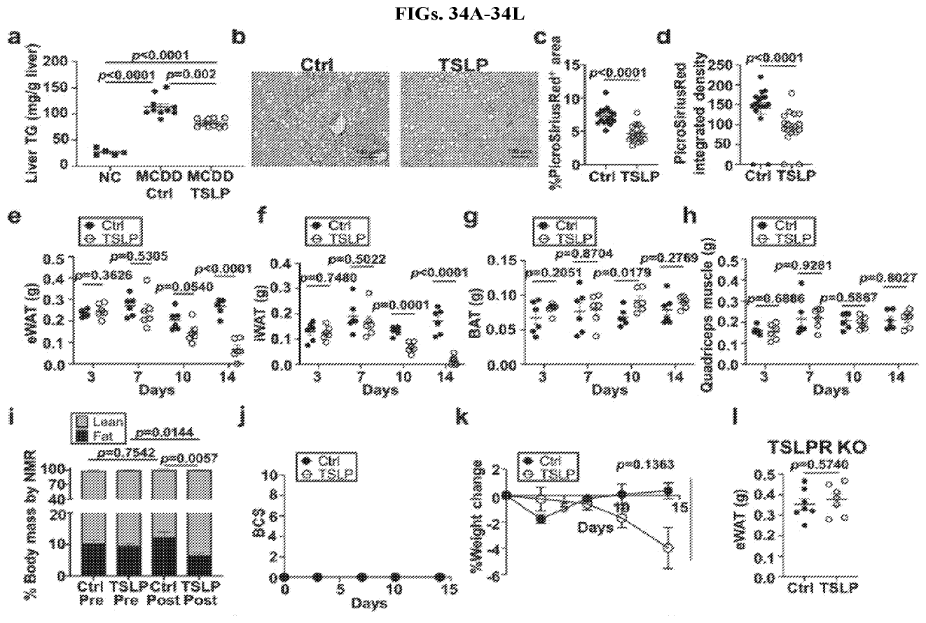

The effect of TSLP on adipose loss was also observed in NC-fed mice. Two weeks after TSLP-AAV injection, NC-fed mice displayed significantly decreased epididymal white adipose tissue (eWAT, visceral fat) and inguinal white adipose tissue (iWAT, subcutaneous fat) mass, without a corresponding decrease in brown adipose tissue (BAT) or muscle mass (FIGS. 29F, 34E-34H). Body composition analysis also showed that TSLP-AAV decreased whole body fat mass percentage (FIG. 34I). Thus, TSLP-AAV-injected mice are not cachexic per se, since there was selective reduction in white fat. Moreover, TSLP-AAV injected mice are not clinically ill, since the body condition scoring (BCS) of TSLP-AAV injected mice remained normal and mice only lost 5% of their total body weight, despite losing a significant amount of white fat (FIGS. 34J-34K). Lastly, the effects of TSLP on white adipose loss are dependent on signaling through the TSLP receptor (TSLPR), as TSLPR KO mice did not lose fat upon TSLP-AAV injection (FIG. 34L).

### T Cells Mediate TSLP-Driven Adipose Loss

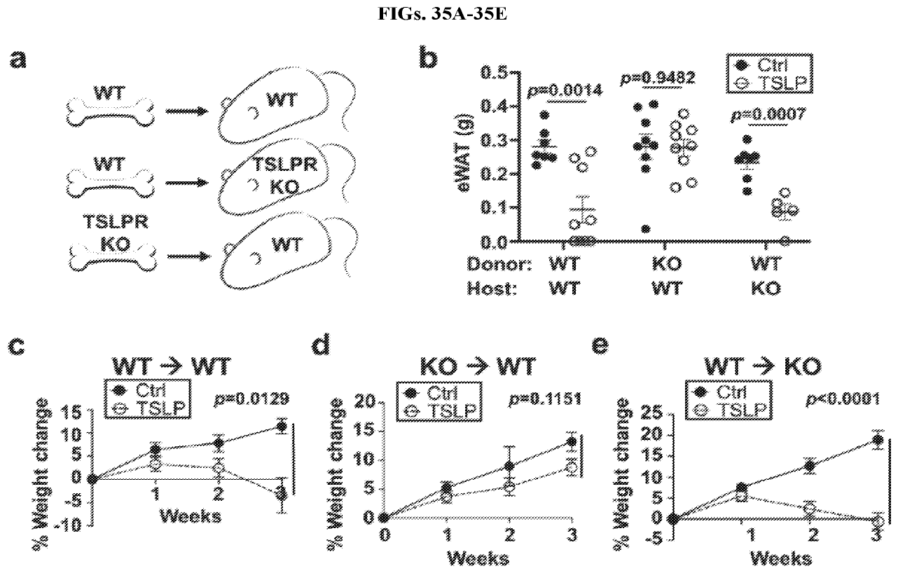

To determine which cell type(s) might be responsible for mediating TSLP-driven white adipose loss, it was first tested whether TSLP causes adipose loss through TSLPR signaling in hematopoietic (radiosensitive) or nonhematopoietic (radioresistant) cells. Three types of bone marrow (BM) irradiated chimeric mice were generated (FIG. 35A) and then injected with Control-AAV or TSLP-AAV after BM reconstitution. TSLP-induced fat loss was observed in TSLPR KO recipient mice transplanted with WT donor BM but not in WT recipient mice transplanted with TSLPR KO donor BM (FIG. 35B), indicating that the effect is mediated by TSLPR signaling in hematopoietic cells. Similar results were seen in HFD-fed BM chimeras (FIGS. 35C-35E).

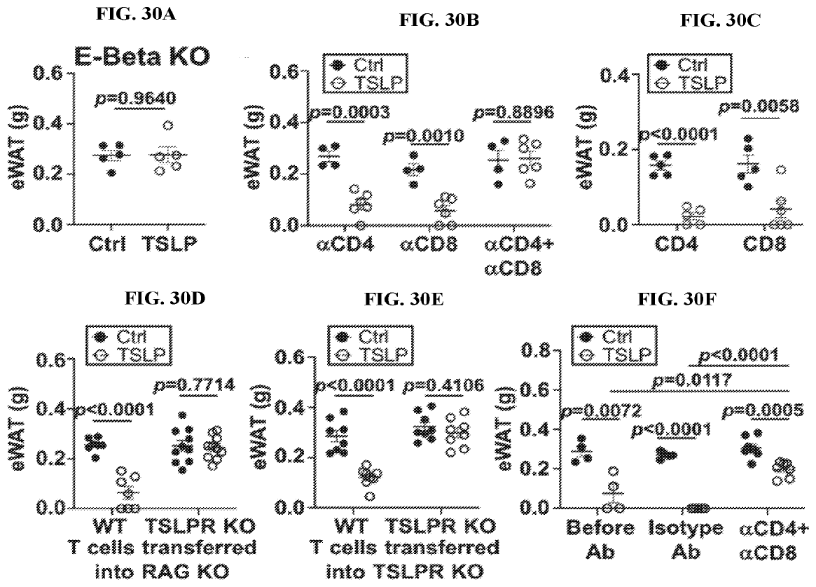

To identify the hematopoietic cell required for TSLP-induced adipose loss, a variety of mouse strains lacking different immune cell types were injected with Control-AAV or TSLP AAV. The injection of TSLP-AAV caused significant fat loss in mice lacking dendritic cells (DCs), eosinophils, or Tregs (FIGS. 36A-36C). However, RAG/common gamma chain DKO (which lack B, T, and innate lymphocytes), RAG KO (which lack B and T cells), and E-Beta KO (which lack αβ T cells) mice did not lose fat upon TSLP-AAV injection (FIGS. 36D-36E and FIG. 30A), suggesting that αβ T cells are necessary for TSLP-induced fat loss. To test whether CD4⁺ or CD8⁺ T cells were necessary for this effect, CD4⁺ and/or CD8⁺ T cells were depleted with antibodies. Surprisingly, it was found that depletion of both CD4⁺ and CD8⁺ T cells was required to prevent TSLP-induced white adipose loss (FIG. 30B), suggesting that either T cell subset is sufficient to induce fat loss in response to TSLP. Accordingly, reconstitution of RAG KO mice with either CD4⁺ or CD8⁺ 115 T cells restored the ability of RAG KO mice to lose fat in response to TSLP (FIG. 30C). Importantly, RAG/common gamma chain DKO mice with selective reconstitution of T cells became susceptible to TSLP-induced fat loss (FIG. 36D), suggesting that T cells but not B cells, NK cells, or ILCs are required for this effect.

To test whether TSLP directly acted on T cells to induce fat loss, WT or TSLPR KO T cells were adoptively transferred into RAG KO mice. TSLP-AAV-injected RAG KO mice that were reconstituted with WT but not TSLPR KO T cells lost white adipose mass (FIG. 30D). Furthermore, TSLPR KO mice adoptively transferred with WT T cells but not TSLPR KO T cells also lost white adipose mass upon TSLP-AAV injection (FIG. 30E). Together, these results indicate that TSLP works directly through the TSLPR on T cells and that TSLPR signaling in T cells is sufficient to mediate TSLP-driven white fat loss. Interestingly, the depletion of T cells at 2 weeks post TSLP-AAV injection allowed partial but significant restoration of white adipose mass, suggesting that T cells are continuously required to maintain the adipose loss in TSLP-AAV-injected mice (FIG. 30F).

### TSLP Increases Sebum Secretion

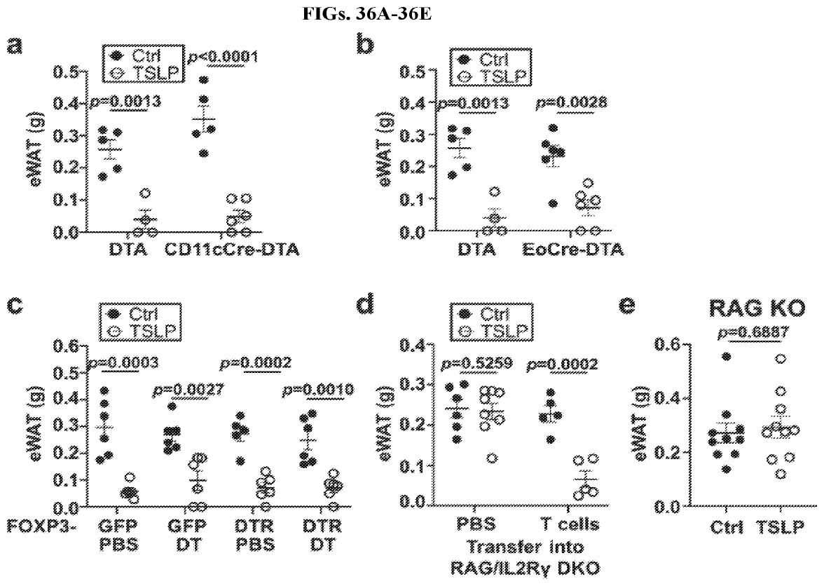

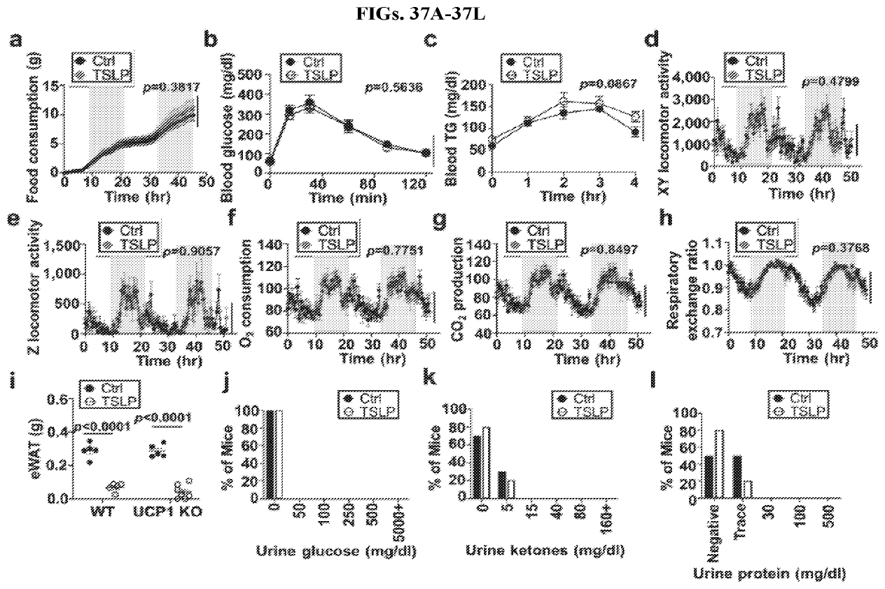

The loss of body weight and white adipose tissue suggests that TSLP causes a net negative energy balance in mice, such that the caloric output is greater than caloric intake. However, decreased caloric intake was not responsible for TSLP-induced fat loss, since TSLP AAV-injected mice consumed more food (FIGS. 31A, 37A), equally absorbed and cleared orally administered glucose and olive oil (FIGS. 37B-37C), and excreted fewer calories/day in their feces as measured by bomb calorimetry (FIG. 31B) compared with Control AAV-injected mice. Since TSLP is a known activator of type II responses, which can cause adipose beiging, it was hypothesized that TSLP causes a net negative energy balance by increasing caloric output through increased thermogenic energy expenditure. Surprisingly, however, there was no evidence of increased energy metabolism in TSLP-AAV-injected mice. Despite losing ˜30% of total fat over the 3-day metabolic cage measurement period, the locomotor activity and the rate of oxygen consumption, carbon dioxide production, energy expenditure, and respiratory exchange ratio were similar between Control-AAV and TSLP-AAV-injected mice (FIGS. 31C, 37D-37H). Consistent with these results, Uncoupled Protein 1 (UCP1) KO mice, which have diminished adipose beiging capacity, were not resistant to TSLP-induced adipose loss (FIG. 37I). Altogether, these data indicated that TSLP does not increase thermogenic energy expenditure.

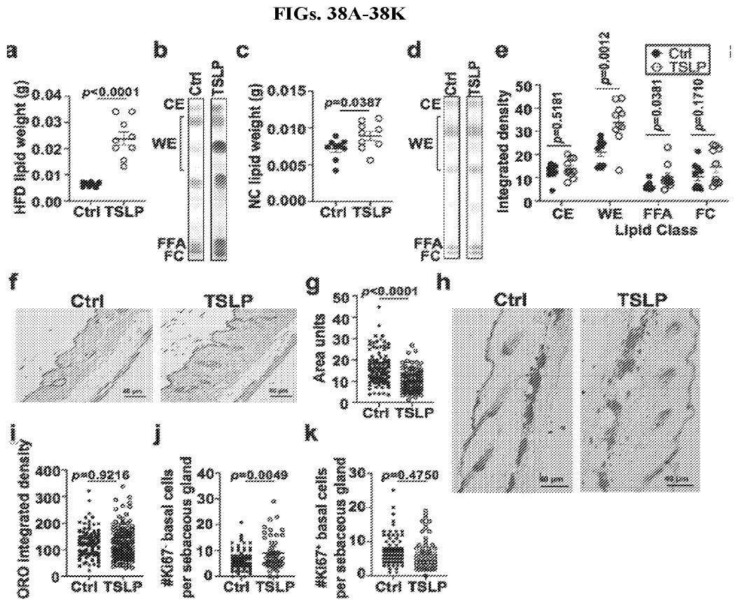

It was next considered that TSLP-AAV-injected mice might be losing energy through the secretion or excretion of calorie-containing metabolites. Fecal caloric content and urine protein, glucose, and ketones were similar between Control-AAV and TSLP-AAV-injected mice (FIGS. 31B, 37J-37L). Intriguingly however, TSLP-AAV-injected mice began to develop a striking grossly visible greasy fur phenotype at ˜4-5 weeks post injection (FIG. 31D). This occurred in both HFD and NC-fed mice, but was more prominent in HFD-fed mice. To determine the identity of the greasy substance on the fur of TSLP-AAV-injected mice, their fur lipids were extracted and analyzed by thin layer chromatography (TLC). Compared to Control-AAV-injected mice, TSLP-AAV-injected HFD-fed mice exhibited significantly increased fur lipid mass (FIG. 38A), which was composed of a mix of wax esters, cholesterol esters, triglycerides, free fatty acids, and free cholesterol (FIGS. 31E, 38B). The increase in wax esters, which are sebum-specific lipids, signified the presence of enhanced sebum secretion in TSLP-AAV-injected mice. Similar to HFD-fed mice, sebum secretion was also increased in TSLP-AAV-injected NC-fed mice at day 10, an early time-point when TSLP-AAV-injected mice are losing white adipose but are not yet grossly visibly greasy (FIGS. 34E-34F, 38C-38E). The histological analysis of the skin of TSLP-AAV-injected mice revealed smaller sebaceous glands with similar total lipid content compared to control mice (FIGS. 38F-38I). Sebum secretion occurs by programmed cell death of sebocytes (holocrine secretion) and release of their intracellular contents into the hair follicle. Thus, although the smaller sebaceous glands seen in TSLP-AAV injected mice was seemingly paradoxical, this was likely a result of increased holocrine secretion and turnover of mature sebocytes. Indeed, an increased fraction of the basal cells lining the sebaceous gland (proliferative sebocyte stem cells) displayed Ki67 positivity within each gland (FIGS. 31F-31G, 38J-38K).

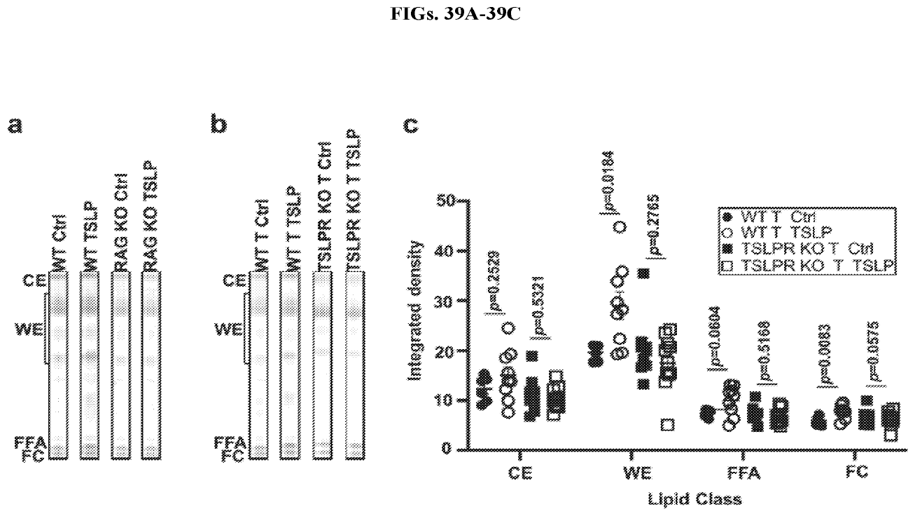

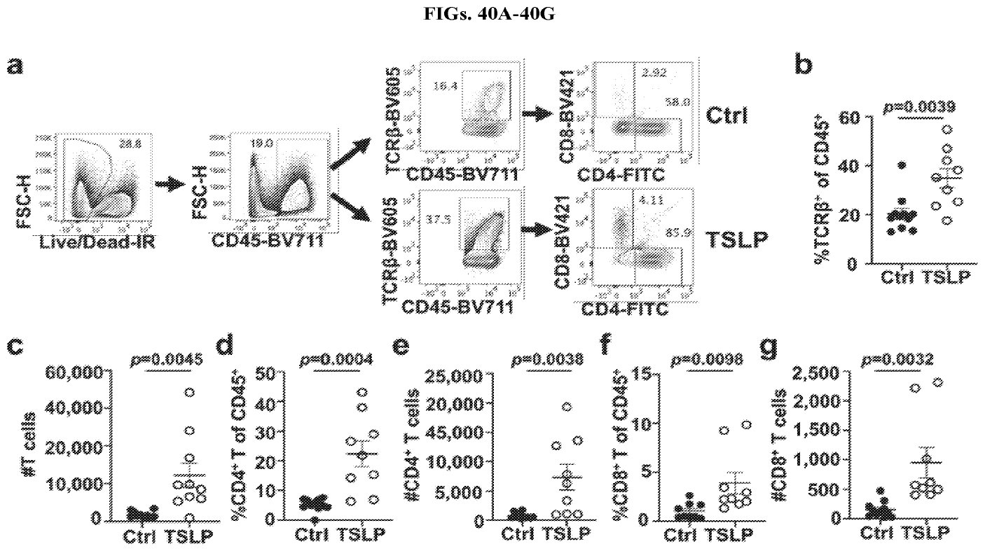

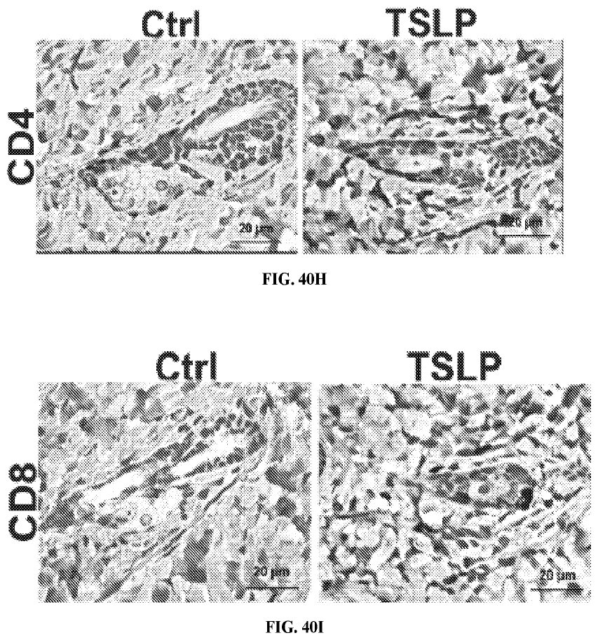

The increased sebum secretion was dependent on TSLPR signaling in T cells, since TSLP-AAV-injected RAG KO mice did not display increased fur wax esters (FIGS. 31H, 39A), whereas RAG KO mice reconstituted with WT T cells but not TSLPR KO T cells displayed more fur lipid wax esters upon TSLP-AAV injection (FIGS. 39B-39C). Flow cytometric and histological examination of the skin showed an increase in CD3⁺, CD4⁺, and CD8⁺ T cells upon TSLP-AAV injection (FIGS. 31I, 40A-40I), which were clustered either in or around sebaceous glands. To test whether sebum secretion was necessary for TSLP mediated white adipose loss, Asebia mice, which lack the enzyme Stearoyl-CoA Desaturase 1 (SCD-1) and have marked sebaceous gland hypoplasia, were used. Asebia mice lost minimal amounts of white fat upon TSLP injection (FIG. 31J), suggesting that sebum secretion is necessary for TSLP-driven fat loss.

### A Homeostatic Role of TSLP in Sebum Secretion

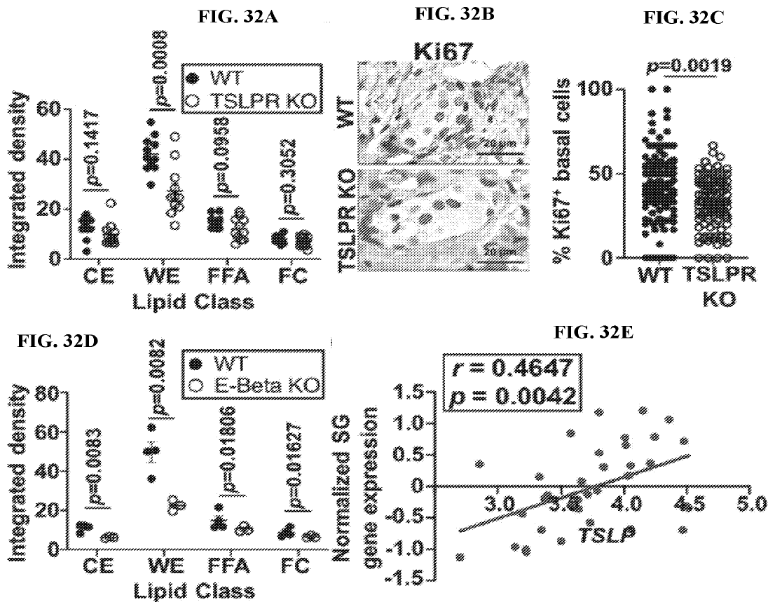

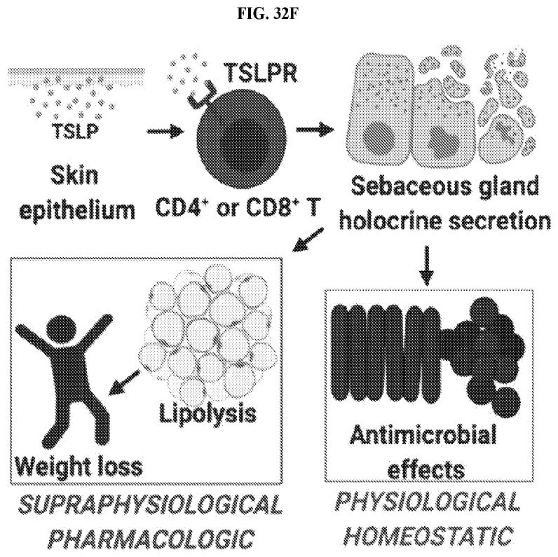

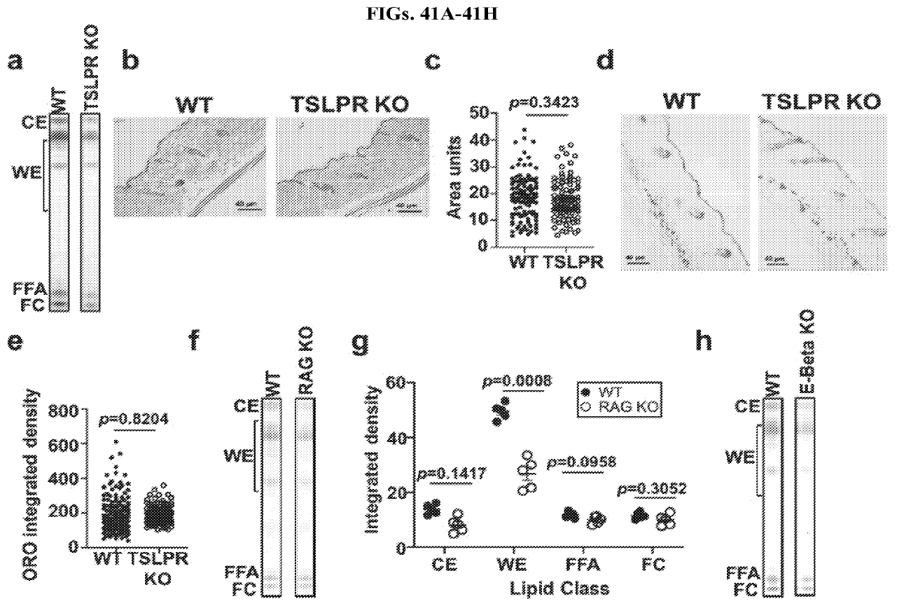

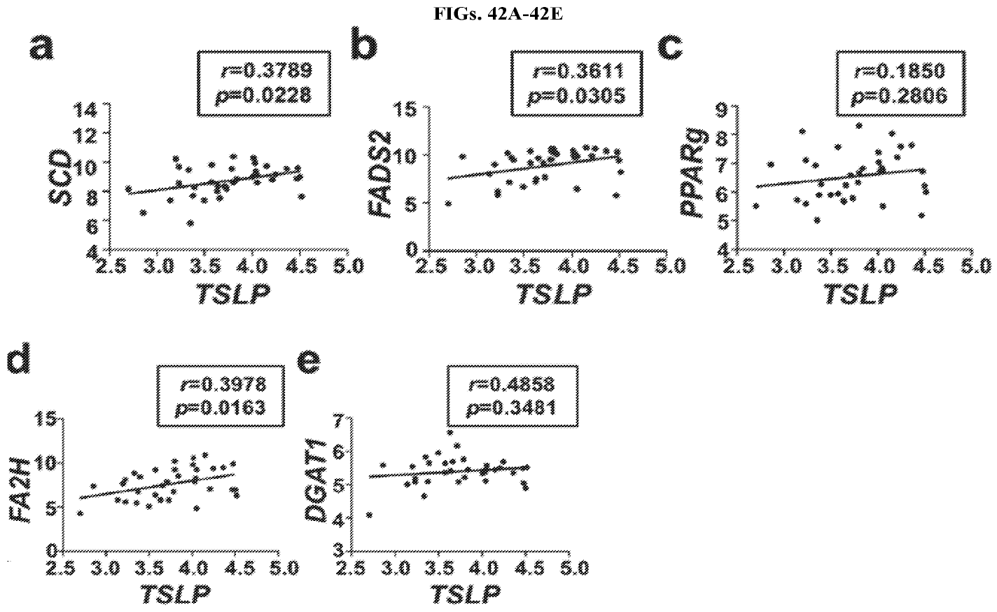

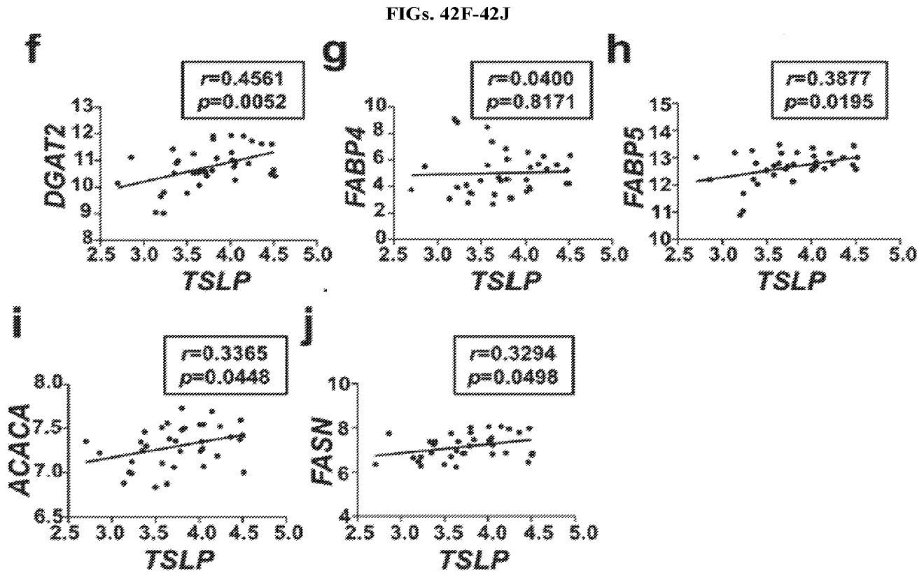

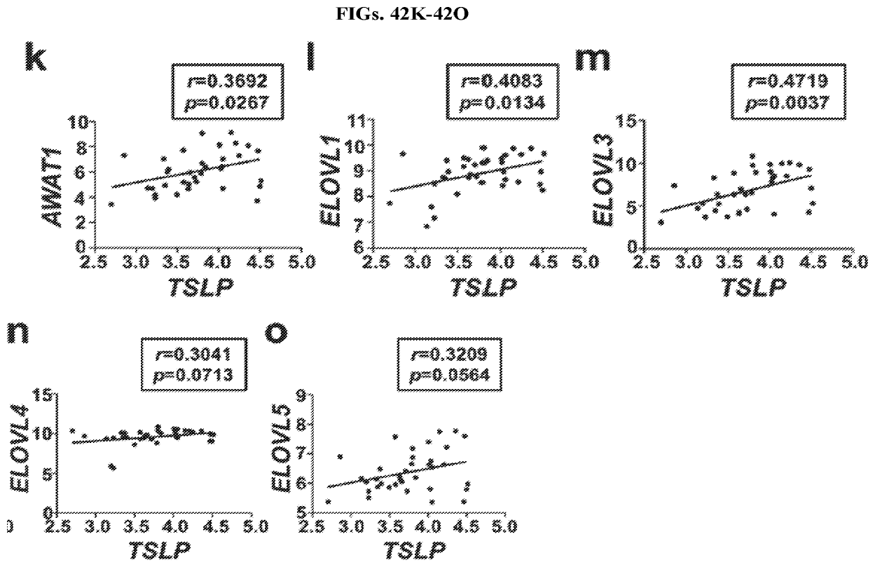

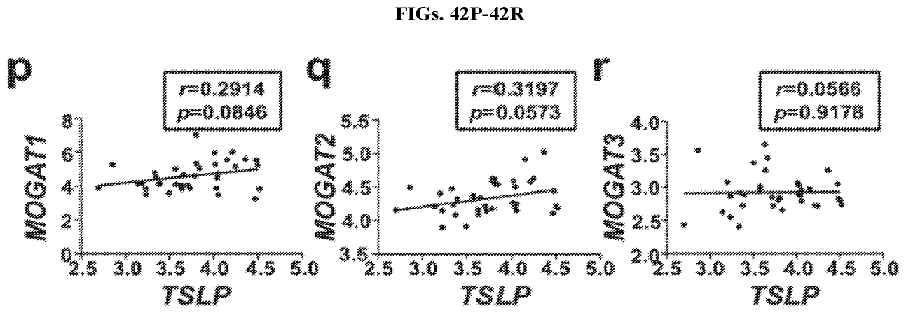

It was then investigated whether TSLP and T cells play a physiological role in the control of sebum secretion. To this end, the fur lipid composition of unmanipulated WT and TSLPR KO mice was examined. Compared to WT mice, TSLPR KO mice displayed decreased fur wax esters and a lower fraction of Ki67+ basal layer cells without differences in sebaceous gland size and lipid content (FIGS. 32A-32C, 41A-41E), indicating that TSLP plays a homeostatic role in sebum secretion. Similarly, unmanipulated RAG KO and E-Beta KO mice showed decreased fur wax esters compared to WT mice (FIGS. 32D, 41F-41H), indicating that T cells also play a homeostatic role in controlling sebum secretion. It was investigated whether this TSLP/sebum axis might also be operational in humans. Examination of TSLP and a panel of sebaceous gland associated genes in publically available data (GSE98774) revealed that expression of TSLP is significantly and positively correlated with expression of sebaceous gland genes in human skin (FIGS. 32E, 42A-42R), indicating that TSLP can homeostatically control sebum production in humans as well.

The results presented here support a model by which TSLP induces selective white adipose loss by directly acting on T cells to induce sebum hypersecretion. The data provide therapeutic proof-of-concept that adipose loss can be achieved by secreting calories from the skin in the form of energy-rich sebum. Physiologically, the results have unraveled unexpected biology relating to how the immune system, specifically TSLP-activated T cells, plays an important role in sebum release. Aside from calories, sebum contains anti-microbial fatty lipids, cathelicidins, bdefensins, and anti-microbial peptides, which form an important mechanical and immuneprotective barrier for the skin. Since TSLP expression in the skin is upregulated by inflammatory stimuli, in certain non-limiting embodiments, the physiological role of TSLP-induced sebum production is not to regulate energy balance but to promote skin barrier function. However, if pharmacologically shifted into high gear, the hypersecretion of sebum can induce systemic fat loss through a pull mechanism (FIG. 32F). Namely, weight loss can be achieved by “sweating fat.”

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a method of treating obesity or an obesity-related disorder, the method comprising: topically administering to the subject a pharmaceutically effective amount of a vitamin D₃ analog.

Embodiment 2 provides the method of embodiment 1, wherein TSLP levels are systemically increased in the subject.

Embodiment 3 provides the method of any one of embodiments 1-2, wherein the TSLP levels are increased by about 5% to about 40% as compared to a control subject.

Embodiment 4 provides the method of any one of embodiments 1-3, wherein the increased TSLP levels result in a reduction of about 5% to about 30% in white adipose tissue in the subject as compared to a control subject.

Embodiment 5 provides the method of any one of embodiments 1-4, wherein the subject experiences weight loss of about 5% to about 30% after a given period.

Embodiment 6 provides the method of any one of embodiments 1-5, wherein the given period is about 1 week to about 12 weeks.

Embodiment 7 provides the method of any one of embodiments 1-6, wherein the obesity-related disorder is at least one disorder selected from nonalcoholic steatohepatitis (NASH), metabolic diseases, type I diabetes, type II diabetes, hypertension, dyslipidemia, coronary heart disease, stroke, gallbladder disease, kidney disease, osteoarthritis, sleep apnea and breathing problems, and cancer.

Embodiment 8 provides the method of any one of embodiments 1-7, wherein the obesity-related disorder is NASH.

Embodiment 9 provides the method of any one of embodiments 1-8, wherein the vitamin D3 analog is selected from the group consisting of 1α,18,25-(OH)3D3; 23-(m-(Dimethylhydroxymethyl)-22-yne-24,25,26,27(tetranor)-1α-OH)2D3; 1α,25-Dihydroxy-trans-Isotachysterol (1,25-trans-Iso-T); (1S,3R,6S)-7,19-Retro-1,25-(OH)2D3; (1S,3R,6R)-7,19-Retro-1,25-(OH)2D3; 22-(p-(Hydroxyphenyl)-23,24,25,26,27-pentanor-D3; 22-(m-(Hydroxyphenyl)-23,24,25,26,27-pentanor-D3; 26,27-cyclo-22-ene-1α,24S-dihydroxyvitamin D3 (MC903 or calcipotriol); 1(S),3(R)-dihydroxy-20(R)-(5′-ethyl-5′-hydroxy-hepta-1′ (E),3′ (E)-dien-1′-yl)-9,10-secopregna-5(Z),7(E),10(19)-triene (EB1089); 1α,25-(OH)-20-epi-22-oxa-24,26,27-trishomovitamin D (KH1060); 22-oxa-1α,25(OH)2D3 (OCT or 22-OXA); 1R,25-dihydroxy-21-(3-hydroxy-3-methylbutyl) vitamin D3, and combinations thereof.

Embodiment 10 provides the method of any one of embodiments 1-9, wherein the vitamin D3 analog is MC903.

Embodiment 11 provides the method of any one of embodiments 1-10, wherein the analog is administered topically to the subject in a dosing schedule wherein a treatment week is followed by a no-treatment week.

Embodiment 12 provides the method of any one of embodiments 1-11, wherein, in the treatment week, the subject is topically administered the analog at a frequency selected from the group consisting of: every day and every other day.

Embodiment 13 provides the method of any one of embodiments 1-12, wherein the analog is the only biologically active agent administered to the subject.

Embodiment 14 provides the method of any one of embodiments 1-13, wherein the administering causes secretion of lipids from the subject's skin.

Embodiment 15 provides a method of treating obesity or an obesity-related disorder, the method comprising: administering to the subject a pharmaceutically effective amount of a TSLP isoform or a viral vector expressing TSLP.

Embodiment 16 provides the method of embodiment 15, whereby TSLP levels are systemically increased in the subject.

Embodiment 17 provides the method of any one of embodiments 15-16, wherein the TSLP isoform is of SEQ ID NO:1, SEQ ID NO:2, and/or SEQ ID NO:8.

Embodiment 18 provides the method of any one of embodiments 15-17, wherein the TSLP isoform is a stabilized isoform.

Embodiment 19 provides the method of any one of embodiments 15-18, wherein the viral vector expressing TSLP comprises an AAV8 vector comprising a TSLP-expression sequence.

Embodiment 20 provides the method of any one of embodiments 15-19, wherein the TSLP-expression sequence is a mouse TSLP sequence or a human TSLP sequence.

Embodiment 21 provides the method of any one of embodiments 15-20, wherein the viral vector comprises a thyroxine binding globulin (TBG) promoter.

Embodiment 22 provides the method of any one of embodiments 15-21, wherein TSLP levels are increased by about 5% to about 40%, relative to a control.

Embodiment 23 provides the method of any one of embodiments 15-22, wherein the subject experiences about a 5% to about 20% reduction in weight over a period of about 1 to 12 weeks.

Embodiment 24 provides the method of any one of embodiments 15-23, wherein the reduction in weight results in substantially no loss of muscle mass.

Embodiment 25 provides the method of any one of embodiments 15-24, wherein the reduction in weight is due to loss of white adipose tissue.

Embodiment 26 provides the method of any one of embodiments 15-25, wherein the administering causes secretion of lipids from the subject's skin.

Embodiment 27 provides the method of any one of embodiments 15-26, wherein the obesity-related disorder is at least one disorder selected from nonalcoholic steatohepatitis (NASH), metabolic diseases, type I diabetes, type II diabetes, hypertension, dyslipidemia, coronary heart disease, stroke, gallbladder disease, kidney disease, osteoarthritis, sleep apnea and breathing problems, and cancer.

Embodiment 28 provides the method of any one of embodiments 15-27, wherein the obesity-related disorder is NASH.

Embodiment 29 provides the method of any one of embodiments 15-28, wherein the administering is by an administration route selected from the group consisting of intravesical, intrapulmonary, intraduodenal, intragastrical, intrathecal, subcutaneous, intramuscular, intradermal, intra-arterial, intravenous, and intrabronchial administration.

Embodiment 30 provides a method of treating a skin disorder or improving scalp health, the method comprising: topically administering to a healthy portion of a subject's skin a pharmaceutically effective amount of a vitamin D₃ analog, wherein the subject is suffering from the skin disorder or needs improvement in scalp health.

Embodiment 31 provides the method of embodiment 30, wherein the skin disorder or improvement in scalp health is selected from the group consisting of eczema, atopic dermatitis, dry skin-associated dermatitis, dry skin (xerosis cutis), ichthyosis (all forms), recurrent skin infections, wrinkles (aging skin), hair loss, and hair growth deficiency.

Embodiment 32 provides the method of any one of embodiments 30-31, wherein the vitamin D₃ analog is administered in a topical composition.

Embodiment 33 provides the method of any one of embodiments 30-32, wherein the vitamin D₃ analog is present in an amount of about 0.0001 to about 10% (w/w).

Embodiment 34 provides the method of any one of embodiments 30-33, wherein the vitamin D₃ analog is selected from the group consisting of 1α,18,25-(OH)₃D₃, 23-(m-(Dimethylhydroxymethyl)-22-yne-24,25,26,27(tetranor)-1α-OH)₂D₃; 1α,25-Dihydroxy-trans-Isotachysterol (1,25-trans-Iso-T); (1S,3R,6S)-7,19-Retro-1,25-(OH)₂D₃; (1S,3R,6R)-7,19-Retro-1,25-(OH)₂D₃; 22-(p-(Hydroxyphenyl)-23,24,25,26,27-pentanor-D₃; 22-(m-(Hydroxyphenyl)-23,24,25,26,27-pentanor-D₃; 26,27-cyclo-22-ene-1α,24S-dihydroxyvitamin D₃ (MC903 or calcipotriol); 1(S),3(R)-dihydroxy-20(R)-(5′-ethyl-5′-hydroxy-hepta-1′(E),3′(E)-dien-1′-yl)-9,10-secopregna-5(Z),7(E),10(19)-triene (EB1089); 1α,25-(OH)-20-epi-22-oxa-24,26,27-trishomovitamin D (KH1060); 22-oxa-1α,25(OH)₂D₃ (OCT or 22-OXA); 1R,25-dihydroxy-21-(3-hydroxy-3-methylbutyl) vitamin D₃, and combinations thereof.

Embodiment 35 provides the method of any one of embodiments 30-34, wherein the vitamin D₃ analog is MC903.

Embodiment 36 provides the method of any one of embodiments 30-35, wherein the vitamin D₃ analog is the only biologically active agent administered to the subject.

Embodiment 37 provides the method of any one of embodiments 30-36, wherein the topical composition is a patch.

Embodiment 38 provides the method of any one of embodiments 30-37, wherein the subject is human.

Embodiment 39 provides a method of treating an eye disorder, the method comprising: topically administering to a subject's skin a pharmaceutically effective amount of a vitamin D₃ analog, wherein the subject is suffering from the eye disorder.

Embodiment 40 provides the method of any embodiment 39, wherein the eye disorders is selected from the group consisting of dry eye syndrome, kerartoconjunctivitis sicca, keratitis sicca, dysfunctional tear syndrome, age-related dry eye syndrome, medication-related dry eye syndrome, menopausal dry eye syndrome, contact lens-associated dry eye, environment-induced dry eye, dysfunctional eyelid-induced dry eye, autoimmune-associated dry eye (Sjogren's syndrome, rheumatoid arthritis, systemic lupus erythematosus), and infection-related conjunctivitis.

Embodiment 41 provides the method of any one of embodiments 39-40, wherein the vitamin D₃ analog is administered in a topical composition.

Embodiment 42 provides the method of any one of embodiments 39-41, wherein the vitamin D₃ analog is present in an amount of about 0.0001 to about 10% (w/w).

Embodiment 43 provides the method of any one of embodiments 39-42, wherein the vitamin D₃ analog is selected from the group consisting of 1α,18,25-(OH)₃D₃, 23-(m-(Dimethylhydroxymethyl)-22-yne-24,25,26,27(tetranor)-1α-OH)₂D₃; 1α,25-Dihydroxy-trans-Isotachysterol (1,25-trans-Iso-T); (1S,3R,6S)-7,19-Retro-1,25-(OH)₂D₃; (1S,3R,6R)-7,19-Retro-1,25-(OH)₂D₃; 22-(p-(Hydroxyphenyl)-23,24,25,26,27-pentanor-D₃; 22-(m-(Hydroxyphenyl)-23,24,25,26,27-pentanor-D₃; 26,27-cyclo-22-ene-1α,24S-dihydroxyvitamin D₃ (MC903 or calcipotriol); 1(S),3(R)-dihydroxy-20(R)-(5′-ethyl-5′-hydroxy-hepta-1′(E),3′(E)-dien-1′-yl)-9,10-secopregna-5(Z),7(E),10(19)-triene (EB1089); 1α,25-(OH)-20-epi-22-oxa-24,26,27-trishomovitamin D (KH1060); 22-oxa-1α,25(OH)₂D₃ (OCT or 22-OXA); 1R,25-dihydroxy-21-(3-hydroxy-3-methylbutyl) vitamin D₃, and combinations thereof.

Embodiment 44 provides the method of any one of embodiments 39-43, wherein the vitamin D₃ analog is MC903.

Embodiment 45 provides the method of any one of embodiments 39-44, wherein the vitamin D₃ analog is the only biologically active agent administered to the subject.

Embodiment 46 provides the method of any one of embodiments 39-45, wherein the vitamin D₃ analog is administered to a portion of the subject's skin without contacting an eye.

Embodiment 47 provides a method of treating, ameliorating, or preventing a skin disorder by reducing or inhibiting sebum release in a subject's skin, the method comprising: administering to a subject in need thereof a pharmaceutically effective amount of a TSLP inhibiting agent.

Embodiment 48 provides the method of embodiment 47, wherein the TSLP inhibiting agent inhibits sfTSLP, lfTSLP, or both sfTSLP and lfTSLP.

Embodiment 49 provides the method of any one of embodiments 47-48, wherein the TSLP is human TSLP.

Embodiment 50 provides the method of any one of embodiments 47-49, wherein the TSLP inhibiting agent is selected from the group consisting of an antibody, a small molecule, siRNA, shRNA, and miRNA.

Embodiment 51 provides the method of any one of embodiments 47-50, wherein the antibody is tezepelumab.

Embodiment 52 provides the method of any one of embodiments 47-51, wherein the skin disorder is acne vulgaris, hidradenitis suppurativa, or seborrheic dermatitis.

Embodiment 53 provides a method of treating, ameliorating, and/or preventing alopecia in a subject, the method comprising topically administering a pharmaceutically effective amount of a vitamin D₃ analog to the subject's skin, wherein the subject is suffering from alopecia or needs improvement in alopecia.

Embodiment 54 provides the method of embodiment 53, wherein the alopecia comprises androgenetic alopecia.

Embodiment 55 provides the method of any one of embodiments 53-54, wherein the administration is to a region of the skin affected by alopecia.

Embodiment 56 provides the method of any one of embodiments 47-54, wherein the administration is to a region of the skin not affected by alopecia.

Embodiment 57 provides the method of any one of embodiments 53-56, wherein the vitamin D₃ analog is administered in a topical composition.

Embodiment 58 provides the method of any one of embodiments 53-57, wherein the vitamin D₃ analog is present in an amount of about 0.0001 to about 10% (w/w).

Embodiment 59 provides the method of any one of embodiments 53-58, wherein the vitamin D₃ analog is selected from the group consisting of 1α,18,25-(OH)₃D₃, 23-(m-(Dimethylhydroxymethyl)-22-yne-24,25,26,27(tetranor)-1α-OH)₂D₃; 1α,25-Dihydroxy-trans-Isotachysterol (1,25-trans-Iso-T); (1S,3R,6S)-7,19-Retro-1,25-(OH)₂D₃; (1S,3R,6R)-7,19-Retro-1,25-(OH)₂D₃; 22-(p-(Hydroxyphenyl)-23,24,25,26,27-pentanor-D₃; 22-(m-(Hydroxyphenyl)-23,24,25,26,27-pentanor-D₃; 26,27-cyclo-22-ene-1α,24S-dihydroxyvitamin D₃ (MC903 or calcipotriol); 1(S),3(R)-dihydroxy-20(R)-(5′-ethyl-5′-hydroxy-hepta-1′(E),3′(E)-dien-1′-yl)-9,10-secopregna-5(Z),7(E),10(19)-triene (EB1089); 1α,25-(OH)-20-epi-22-oxa-24,26,27-trishomovitamin D (KH1060); 22-oxa-1α,25(OH)₂D₃ (OCT or 22-OXA); 1R,25-dihydroxy-21-(3-hydroxy-3-methylbutyl) vitamin D₃, and combinations thereof.

Embodiment 60 provides the method of any one of embodiments 53-59, wherein the vitamin D₃ analog is MC903.

Embodiment 61 provides the method of any one of embodiments 53-60, wherein the vitamin D₃ analog is the only biologically active agent administered to the subject.

Embodiment 62 provides the method of any one of embodiments 53-61, wherein the topical composition is a patch.

Embodiment 63 provides the method of any one of embodiments 53-62, wherein the subject is human.

1. A method of treating or ameliorating obesity or an obesity-related disorder, the method comprising topically administering to the subject a pharmaceutically effective amount of a vitamin D₃ analog.

2. The method of claim 1, wherein at least one of the following applies:
(a) the administering systemically increases TSLP levels in the subject,
optionally wherein the TSLP levels are increased by about 5% to about 40% as compared to a control subject;
(b) the increased TSLP levels result in a reduction of about 5% to about 30% in white adipose tissue in the subject as compared to a control subject;
(c) the subject experiences weight loss of about 5% to about 30% after a given period, optionally wherein the given period is about 1 week to about 12 weeks;
(d) the analog is administered topically to the subject in a dosing schedule wherein a treatment week is followed by a no-treatment week,
optionally wherein, in the treatment week, the subject is topically administered the analog at a frequency selected from the group consisting of: every day and every other day;
(e) the vitamin D₃ analog is the only biologically active agent administered to the subject to treat or ameliorate the obesity or obesity-related disorder;
(f) the administering causes secretion of lipids from the subject's skin;
(g) the obesity-related disorder is at least one disorder selected from nonalcoholic steatohepatitis (NASH), metabolic diseases, type I diabetes, type II diabetes, hypertension, dyslipidemia, coronary heart disease, stroke, gallbladder disease, kidney disease, osteoarthritis, sleep apnea and breathing problems, and cancer;
(h) the vitamin D₃ analog is selected from the group consisting of 1α,18,25-(OH)₃D₃; 23-(m-(Dimethylhydroxymethyl)-22-yne-24,25,26,27(tetranor)-1α-OH)₂D₃; 1α,25-Dihydroxy-trans-Isotachysterol (1,25-trans-Iso-T); (1S,3R,6S)-7,19-Retro-1,25-(OH)₂D₃; (1S,3R,6R)-7,19-Retro-1,25-(OH)₂D₃; 22-(p-(Hydroxyphenyl)-23,24,25,26,27-pentanor-D₃; 22-(m-(Hydroxyphenyl)-23,24,25,26,27-pentanor-D₃; 26,27-cyclo-22-ene-1α,24S-dihydroxyvitamin D₃ (MC903 or calcipotriol); 1(S),3(R)-dihydroxy-20(R)-(5′-ethyl-5′-hydroxy-hepta-1′(E),3′(E)-dien-1′-yl)-9,10-secopregna-5(Z),7(E),10(19)-triene EB1089); 1α,25-(OH)-20-epi-22-oxa-24,26,27-trishomovitamin D (KH1060); 22-oxa-1α,25(OH)₂D₃ (OCT or 22-OXA); 1R,25-dihydroxy-21-(3-hydroxy-3-methylbutyl) vitamin D₃, and combinations thereof.

3-7. (canceled)

8. The method of claim 1, wherein the obesity-related disorder is NASH.

9. (canceled)

10. The method of claim 1, wherein the vitamin D₃ analog is MC903.

11-14. (canceled)

15. A method of treating or ameliorating obesity or an obesity-related disorder, the method comprising administering to the subject a pharmaceutically effective amount of a TSLP isoform or a viral vector expressing TSLP.

16. The method of claim 15, whereby at least one of the following applies:
(a) the administering systemically increases TSLP levels in the subject;
(b) the TSLP isoform is of SEQ ID NO:1, SEQ ID NO:2, or SEQ ID NO:8;
(c) the TSLP isoform is a stabilized isoform;
(d) the viral vector expressing TSLP comprises an AAV8 vector comprising a TSLP-expression sequence;
(e) TSLP levels are increased by about 5% to about 40%, relative to a control;
(f) the obesity-related disorder is at least one disorder selected from nonalcoholic steatohepatitis (NASH), metabolic diseases, type I diabetes, type II diabetes, hypertension, dyslipidemia, coronary heart disease, stroke, gallbladder disease, kidney disease, osteoarthritis, sleep apnea and breathing problems, and cancer;
(g) the subject experiences about a 5% to about 20% reduction in weight over a period of about 1 to 12 weeks;
(h) the reduction in weight results in substantially no loss of muscle mass;
(i) the reduction in weight is due to loss of white adipose tissue;
(j) the administering causes secretion of lipids from the subject's skin;
(k) the administering is by an administration route selected from the group consisting of intravesical, intrapulmonary, intraduodenal, intragastrical, intrathecal, subcutaneous, intramuscular, intradermal, intra-arterial, intravenous, and intrabronchial administration.

17-19. (canceled)

20. The method of claim 16, wherein at least one of the following applies:
(a) the TSLP-expression sequence is a mouse TSLP sequence or a human TSLP sequences;
(b) the viral vector comprises a thyroxine binding globulin (TBG) promoter.

21-27. (canceled)

28. The method of claim 15, wherein the obesity-related disorder is NASH.

29. (canceled)

30. A method of treating or ameliorating a skin disorder or improving scalp health, the method comprising topically administering to a healthy portion of a subject's skin a pharmaceutically effective amount of a vitamin D₃ analog, wherein the subject is suffering from the skin disorder or needs improvement in scalp health.

31. The method of claim 30, wherein the skin disorder or improvement in scalp health is selected from the group consisting of eczema, atopic dermatitis, dry skin-associated dermatitis, dry skin (xerosis cutis), ichthyosis (all forms), recurrent skin infections, wrinkles (aging skin), hair loss, and hair growth deficiency.

32. The method of claim 30, wherein at least one of the following applies:
(a) the vitamin D₃ analog is administered in a topical composition;
(b) the vitamin D₃ analog is selected from the group consisting of 1α,18,25-(OH)₃D₃; 23-(m-(Dimethylhydroxymethyl)-22-yne-24,25,26,27(tetranor)-1α-OH)₂D₃; 1α,25-Dihydroxy-trans-Isotachysterol (1,25-trans-Iso-T); (1S,3R,6S)-7,19-Retro-1,25-(OH)₂D₃; (1S,3R,6R)-7,19-Retro-1,25-(OH)₂D₃; 22-(p-(Hydroxyphenyl)-23,24,25,26,27-pentanor-D₃; 22-(m-(Hydroxyphenyl)-23,24,25,26,27-pentanor-D₃; 26,27-cyclo-22-ene-1α,24S-dihydroxyvitamin D₃ (MC903 or calcipotriol); 1(S),3(R)-dihydroxy-20(R)-(5′-ethyl-5′-hydroxy-hepta-1′ (E),3′ (E)-dien-1′-yl)-9,10-secopregna-5(Z),7(E),10(19)-triene EB1089); 1α,25-(OH)-20-epi-22-oxa-24,26,27-trishomovitamin D (KH1060); 22-oxa-1α,25(OH)₂D₃ (OCT or 22-OXA); 1R,25-dihydroxy-21-(3-hydroxy-3-methylbutyl) vitamin D₃, and combinations thereof;
(c) the vitamin D₃ analog is present in an amount of about 0.0001 to about 10% (w/w);
(d) the vitamin D₃ analog is the only biologically active agent administered to the subject to treat or ameliorate the skin disorder or improve scalp health;
(e) the topical composition is a patch;
(f) to the subject is human.

33-34. (canceled)

35. The method of claim 32, wherein the vitamin D₃ analog is MC903.

36-38. (canceled)

39. A method of treating or ameliorating an eye disorder, the method comprising topically administering to the skin of a subject suffering from the eye disorder a pharmaceutically effective amount of a vitamin D₃ analog.

40. The method of claim 39, wherein the eye disorder is selected from the group consisting of dry eye syndrome, keraftoconjunctivitis sicca, keratitis sicca, dysfunctional tear syndrome, age-related dry eye syndrome, medication-related dry eye syndrome, menopausal dry eye syndrome, contact lens-associated dry eye, environment-induced dry eye, dysfunctional eyelid-induced dry eye, autoimmune-associated dry eye, and infection-related conjunctivitis.

41. The method of claim 39, wherein at least one of the following applies:
(a) the vitamin D₃ analog is administered in a topical composition;
(a) the vitamin D₃ analog is selected from the group consisting of 1α,18,25-(OH)₃D₃; 23-(m-(Dimethylhydroxymethyl)-22-yne-24,25,26,27(tetranor)-1α-OH)₂D₃; 1α,25-Dihydroxy-trans-Isotachysterol (1,25-trans-Iso-T); (1S,3R,6S)-7,19-Retro-1,25-(OH)₂D₃; (1S,3R,6R)-7,19-Retro-1,25-(OH)₂D₃; 22-(p-(Hydroxyphenyl)-23,24,25,26,27-pentanor-D₃; 22-(m-(Hydroxyphenyl)-23,24,25,26,27-pentanor-D₃; 26,27-cyclo-22-ene-1α,24S-dihydroxyvitamin D₃ (MC903 or calcipotriol); 1(S),3(R)-dihydroxy-20(R)-(5′-ethyl-5′-hydroxy-hepta-1′(E),3′(E)-dien-1′-yl)-9,10-secopregna-5(Z),7(E),10(19)-triene (EB1089); 1α,25-(OH)-20-epi-22-oxa-24,26,27-trishomovitamin D (KH1060); 22-oxa-1α,25(OH)₂D₃ (OCT or 22-OXA); 1R,25-dihydroxy-21-(3-hydroxy-3-methylbutyl) vitamin D₃, and combinations thereof;
(c) the vitamin D₃ analog is present in an amount of about 0.0001 to about 10% (w/w);
(d) the vitamin D₃ analog is the only biologically active agent administered to the subject to treat or ameliorate the eye disorder;
(e) the vitamin D₃ analog is administered to a portion of the subject's skin without contacting an eye.

42-43. (canceled)

44. The method of claim 41, wherein the vitamin D₃ analog is MC903.

45-46. (canceled)

47. A method of treating, ameliorating, or preventing a skin disorder by reducing or inhibiting sebum release in a subject's skin, the method comprising administering to a subject in need thereof a pharmaceutically effective amount of a TSLP inhibiting agent.

48. The method of claim 47, wherein at least one of the following applies:
(a) the TSLP inhibiting agent inhibits sfTSLP, lfTSLP, or both sfTSLP and lfTSLP;
(b) the TSLP inhibiting agent is selected from the group consisting of an antibody, a small molecule, siRNA, shRNA, and miRNA;
(c) the TSLP inhibiting agent is tezepelumab;
(d) the TSLP is human TSLP;
(e) the skin disorder is acne vulgaris, hidradenitis suppurativa, or seborrheic dermatitis.

49-52. (canceled)

53. A method of treating, ameliorating, or preventing alopecia in a subject, the method comprising topically administering a pharmaceutically effective amount of a vitamin D₃ analog to the subject's skin, wherein the subject is suffering from alopecia or needs improvement in alopecia.

54. The method of claim 53, wherein at least one of the following applies:
(a) the alopecia comprises androgenetic alopecia;
(b) the administration is to a region of the skin affected by alopecia;
(c) the administration is to a region of the skin not affected by alopecia;
(d) the vitamin D₃ analog is selected from the group consisting of 1α,18,25-(OH)₃D₃; 23-(m-(Dimethylhydroxymethyl)-22-yne-24,25,26,27(tetranor)-1α-OH)₂D₃; 1α,25-Dihydroxy-trans-Isotachysterol (1,25-trans-Iso-T); (1S,3R,6S)-7,19-Retro-1,25-(OH)₂D₃; (1S,3R,6R)-7,19-Retro-1,25-(OH)₂D₃; 22-(p-(Hydroxyphenyl)-23,24,25,26,27-pentanor-D₃; 22-(m-(Hydroxyphenyl)-23,24,25,26,27-pentanor-D₃; 26,27-cyclo-22-ene-1α,24S-dihydroxyvitamin D₃ (MC903 or calcipotriol); 1(S),3(R)-dihydroxy-20(R)-(5′-ethyl-5′-hydroxy-hepta-1′(E),3′(E)-dien-1′-yl)-9,10-secopregna-5(Z),7(E),10(19)-triene EB1089); 1α,25-(OH)-20-epi-22-oxa-24,26,27-trishomovitamin D (KH1060); 22-oxa-1α,25(OH)₂D₃ (OCT or 22-OXA); 1R,25-dihydroxy-21-(3-hydroxy-3-methylbutyl) vitamin D₃, and combinations thereof;
(e) the vitamin D₃ analog is administered in a topical composition;
(f) the vitamin D₃ analog is present in an amount of about 0.0001 to about 10% (w/w);
(g) the vitamin D₃ analog is the only biologically active agent administered to the subject to treat, ameliorate, or prevent alopecia;
(h) the topical composition is a patch;
(i) the subject is human.

55-59. (canceled)

60. The method of claim 53, wherein the vitamin D₃ analog is MC903.

61-63. (canceled)


SEQUENCE LISTING (8 sequences)

SEQ ID NO: 1 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 2 - protein, 63 aa, Homo sapiens
MFAMKTKAALAIWCPGYSETQINATQAMKKRRKRKVTTNKCLEQVSQLQGLWRRFNRPLLKQQ

SEQ ID NO: 3 - DNA, 480 bp, Homo sapiens
atgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 4 - protein, 159 aa, Homo sapiens; identical to SEQ ID NO: 1

SEQ ID NO: 5 - DNA, 192 bp, Homo sapiens
atgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 6 - protein, 63 aa, Homo sapiens; identical to SEQ ID NO: 2

SEQ ID NO: 7 - DNA, 423 bp, Homo sapiens
atggttcttctcaggagcctcttcatcctgcaagtactagtacggatggggctaacttacaacttttctaactgcaacttcacgtcaattacgaaaatatattgtaacataatttttcatgacctgactggagatttgaaaggggctaagttcgagcaaatcgaggactgtgagagcaagccagcttgtctcctgaaaatcgagtactatactctcaatcctatccctggctgcccttcactccccgacaaaacatttgcccggagaacaagagaagccctcaatgaccactgcccaggctaccctgaaactgagagaaatgacggtactcaggaaatggcacaagaagtccaaaacatctgcctgaatcaaacctcacaaattctaagattgtggtattccttcatgcaatctccagaataa

SEQ ID NO: 8 - protein, 140 aa, Homo sapiens
MVLLRSLFILQVLVRMGLTYNFSNCNFTSITKIYCNIIFHDLTGDLKGAKFEQIEDCESKPACLLKIEYYTLNPIPGCPSLPDKTFARRTREALNDHCPGYPETERNDGTQEMAQEVQNICLNQTSQILRLWYSFMQSPE